SEQUENCE LISTING (82 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - DNA, 1484 bp, Homo sapiens
gtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 3 - protein, 355 aa, Macaca fascicularis
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 4 - DNA, 1068 bp, Macaca fascicularis
atggattatacacttgaccccagcatgacaacaatgaccgactactactaccctgatagcctctcaagcccctgtgatggagaacttatccagagaaacgacaagttgctccttgctgtcttttattgcctcctgtttgtattcagtcttctgggaaacagcctggtcatcctggtccttgtggtctgcaagaagctgaggaacatcacagacatatacctcttgaacctggccctgtctgacctgctttttgtcttctccttcccctttcagacctactatcagctggatcagtgggtgtttgggactgtaatgtgcaaagtggtgtctggcttttattacattggcttctacagcagcatgtttttcatcaccctcatgagtgtggacaggtacctggctgttgtccatgccgtgtatgccataaaagtgaggacgatcaggatgggcacaaccctgagcctggtagtatggctaaccgccattatggctaccatcccattgctagtgttttaccaagtggcctctgaagatggtgttctacagtgttattcattttacaatcaacagactttgaagtggaagatcttcaccaactttgaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagtcaacaactgaattatgccacccatgtcacagaaatcatttcctttactcactgctgtgtgaaccctgttatctatgcttttgtaggggagaagttcaagaaacacctctcagaaatatttcagaaaagttgcagccatatcttcatctacctaggaagacaaatgcctagggagagctgtgaaaagtcatcatcctgccagcagcactccttccgttcctccagcatagactacattttgtga

SEQ ID NO: 5 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 6 - DNA, 1156 bp, Mus musculus
tggcagaggagtgggcagctctgaaacctcagaagaaaggctcgctcagataattggtcttcctgcctcgatggattacacgatggagcccaacgtcacgatgaccgactactaccctgatttcttcaccgccccctgtgacgcagagttcctcctcaggggcagcatgctgtatctggccatcttgtactgcgtcttgtttgtgctgggccttctggggaacagcctggtcatcttagtcctcgtgggctgcaagaaactgaggagcatcacagatatctacctcctgaacctggccgcatccgacctgctctttgtcctctctattccttttcagacccacaacctgctggaccagtgggtgtttgggactgcgatgtgtaaggtggtctctggcctttattacattggttttttcagcagtatgttcttcatcaccctaatgagtgtggacaggtatctggctattgtccacgctgtctatgccatcaaggtgaggacggccagcgtgggcacagccctgagtctgacagtgtggctggctgctgtcacagccaccatccccttgatggttttttaccaagtggcctctgaagacggcatgctacaatgtttccagttttatgaagagcagtctttgaggtggaagctctttacccactttgaaatcaacgccttgggtctgctgctcccctttgccatcctcctgttctgctatgtcaggatcctgcagcagctgcggggctgcctgaaccacaacaggaccagagccatcaagctggtgctcaccgtagtcattgtgtctttactcttctgggtcccattcaacgtggcccttttcctcacgtccctgcacgacctgcacatcttggatggatgtgccacgaggcagaggctggctctggccatccatgtcacagaggtcatctcttttacccactgctgcgtgaaccccgtcatctacgcgttcataggagagaagtttaagaaacacctcatggatgtgtttcaaaagagctgcagccacatcttcctctacttagggagacaaatgcccgtgggggcgttggaaaggcagctgtcctcgaaccagcgatcttcccattcttccaccctggatgacatcttgtaaggggagtgtgcagggcaggcagac

SEQ ID NO: 7 - protein, 353 aa, Rattus norvegicus
MDYTLEPNVTMTDYYPDFFTTPCDTELLLRGGTLYLAVLYCILFVLGLLGNSLVILVLVACKKLRSITDVYLLNLAASDLLFVLSIPFQTHNLLDQWVFGTVMCKVVSGLYYIGFFSSMLFITLMSVDRYLAVVHPVHAIKVRTARVGTALSLAVWLAAIAATVPLMVFYQVSSEDGMLQCFQLYDEQSLRWKLFTHFEVNALGLLLPFAILLFCYVRILQQLRGCLNHNRTRAIKLVLTIVVVSLLFWVPFNVVLFLTSLHDMHILEGCATRQRLALATHVTEVISFMHCCVNPVIYAFIGEKFKKHLVDVFQKSCSHIFLYVGRQMPVGALERQLSSNQRSSHSSTLDYIL

SEQ ID NO: 8 - DNA, 1215 bp, Rattus norvegicus
cagacatgcggcagaggagtgggcagctctgaaacctcagaaggaaggctcgctcacctacctggttttcccgcctcgatggattacacgttggagcccaatgtcacgatgactgactactaccctgacttcttcaccaccccctgtgacacagagctcctcctcaggggtggcacgttgtatctggccgtcttatactgcatcttgtttgtgctgggccttctgggaaacagcctggtcatcttggtccttgtggcctgcaagaaactgaggagtatcacggacgtctacctcctgaacctggccgcttctgacctgctcttcgtcctctccattccctttcagacccacaacctgctggaccagtgggtgtttgggaccgtgatgtgtaaggtggtctctggcctctactacattggcttcttcagcagcatgctcttcatcaccctcatgagtgtggacaggtacctggctgtcgtccaccctgtccatgccatcaaagtgaggacggccagagtgggcacagccctgagcctggcagtgtggctggctgccatcgcggccaccgtcccactgatggttttttaccaggtgtcctctgaagacggcatgctacagtgcttccaactttacgacgagcagtctctgaggtggaagctcttcacccactttgaagtcaacgccttgggtctgctgctcccctttgccatcctcctgttctgctacgtcaggatcctgcagcagctgcggggttgcctgaaccacaacaggaccagagccatcaaactggtgctcaccatagtcgtcgtgtctttactcttctgggtcccattcaacgtggtcctcttcctcacgtccctgcacgacatgcacatcttggagggatgtgccaccaggcagaggctggccctggccacccacgtcacggaggtcatctctttcatgcattgctgcgtgaaccctgtcatctatgctttcatcggagagaagttcaagaagcacctcgtggatgtgtttcaaaagagctgcagccacatcttcctctacgtcgggagacagatgccagtgggggcgttggaaaggcaactgtcctcgaaccagcgatcttcccactcttccacactggactacatcttgtaaggggggtggtgtgcacgacaggcagcctccacctacattgcccttcctgctcccaatcttctccccccacctccc

SEQ ID NO: 9 - protein, 10 aa, Artificial Sequence
  source: /note=“Description of Artificial Sequence Synthetic peptide”
GFTFSSYAMH

SEQ ID NO: 10 - protein, 18 aa, Artificial Sequence
  source: /note=“Description of Artificial Sequence Synthetic peptide”
AVISYDGSNKYYADSVKG

SEQ ID NO: 11 - protein, 10 aa, Artificial Sequence
  source: /note=“Description of Artificial Sequence Synthetic peptide”
ARVRDRAFDI

SEQ ID NO: 12 - protein, 14 aa, Artificial Sequence
  source: /note=“Description of Artificial Sequence Synthetic peptide”
TLRSGINVGTYRIY

SEQ ID NO: 13 - protein, 11 aa, Artificial Sequence
  source: /note=“Description of Artificial Sequence Synthetic peptide”
YKSDSDKQQGS

SEQ ID NO: 14 - protein, 9 aa, Artificial Sequence
  source: /note=“Description of Artificial Sequence Synthetic peptide”
WHSSARNWV

SEQ ID NO: 15 - protein, 5 aa, Artificial Sequence
  source: /note=“Description of Artificial Sequence Synthetic peptide”
SYGMH

SEQ ID NO: 16 - protein, 17 aa, Artificial Sequence
  source: /note=“Description of Artificial Sequence Synthetic peptide”
VISYDGSNKYYADSVKG

SEQ ID NO: 17 - protein, 10 aa, Artificial Sequence
  source: /note=“Description of Artificial Sequence Synthetic peptide”
DRRGGGYGDY

SEQ ID NO: 18 - protein, 25 aa, Artificial Sequence
  source: /note=“Description of Artificial Sequence Synthetic peptide”
QVQLVESGGGVVQPGRSLRLSCAAS

SEQ ID NO: 19 - protein, 13 aa, Artificial Sequence
  source: /note=“Description of Artificial Sequence Synthetic peptide”
WVRQAPGKGLEWV

SEQ ID NO: 20 - protein, 11 aa, Artificial Sequence
  source: /note=“Description of Artificial Sequence Synthetic peptide”
MIWHSSARNWV

SEQ ID NO: 21 - protein, 5 aa, Artificial Sequence
  source: /note=“Description of Artificial Sequence Synthetic peptide”
SYAMH

SEQ ID NO: 22 - protein, 30 aa, Artificial Sequence
  source: /note=“Description of Artificial Sequence Synthetic polypeptide”
RFTISRDNSKNTLYLQMNSLRAEDTAVYYC

SEQ ID NO: 23 - protein, 8 aa, Artificial Sequence
  source: /note=“Description of Artificial Sequence Synthetic peptide”
VRDRAFDI

SEQ ID NO: 24 - protein, 11 aa, Artificial Sequence
  source: /note=“Description of Artificial Sequence Synthetic peptide”
WGQGTMVTVSS

SEQ ID NO: 25 - protein, 22 aa, Artificial Sequence
  source: /note=“Description of Artificial Sequence Synthetic peptide”
QAVLTQPASLSASPGASASLTC

SEQ ID NO: 26 - protein, 15 aa, Artificial Sequence
  source: /note=“Description of Artificial Sequence Synthetic peptide”
WYQQKPGSPPQYLLR

SEQ ID NO: 27 - protein, 5 aa, Artificial Sequence
  source: /note=“Description of Artificial Sequence Synthetic peptide”
SNYMS

SEQ ID NO: 28 - protein, 16 aa, Artificial Sequence
  source: /note=“Description of Artificial Sequence Synthetic peptide”
VIYSGGSTYYADSVKG

SEQ ID NO: 29 - protein, 7 aa, Artificial Sequence
  source: /note=“Description of Artificial Sequence Synthetic peptide”
GLGSADY

SEQ ID NO: 30 - protein, 16 aa, Artificial Sequence
  source: /note=“Description of Artificial Sequence Synthetic peptide”
RSSQSLLHSNGNTYLN

SEQ ID NO: 31 - protein, 7 aa, Artificial Sequence
  source: /note=“Description of Artificial Sequence Synthetic peptide”
KVSIRDS

SEQ ID NO: 32 - protein, 9 aa, Artificial Sequence
  source: /note=“Description of Artificial Sequence Synthetic peptide”
MQSTQWPIT

SEQ ID NO: 33 - protein, 34 aa, Artificial Sequence
  source: /note=“Description of Artificial Sequence Synthetic polypeptide”
GVPSRFSGSKDASANAGILLISGLQSEDEADYYC

SEQ ID NO: 34 - protein, 11 aa, Artificial Sequence
  source: /note=“Description of Artificial Sequence Synthetic peptide”
FGGGTKLTVLG

SEQ ID NO: 35 - protein, 30 aa, Artificial Sequence
  source: /note=“Description of Artificial Sequence Synthetic polypeptide”
EVQLVESGGGVVQPGRSLRLSCAASGFTFS

SEQ ID NO: 36 - protein, 14 aa, Artificial Sequence
  source: /note=“Description of Artificial Sequence Synthetic peptide”
WVRQAPGKGLEWVA

SEQ ID NO: 37 - protein, 13 aa, Artificial Sequence
  source: /note=“Description of Artificial Sequence Synthetic peptide”
IIKSGSSDKQQGS

SEQ ID NO: 38 - protein, 32 aa, Artificial Sequence
  source: /note=“Description of Artificial Sequence Synthetic polypeptide”
RFTISRDNSKNTLYLQMNSLRAEDTAVYYCAK

SEQ ID NO: 39 - protein, 11 aa, Artificial Sequence
  source: /note=“Description of Artificial Sequence Synthetic peptide”
WGQGTLVTVSS

SEQ ID NO: 40 - protein, 10 aa, Artificial Sequence
  source: /note=“Description of Artificial Sequence Synthetic peptide”
FGGGTQLTVL

SEQ ID NO: 41 - protein, 30 aa, Artificial Sequence
  source: /note=“Description of Artificial Sequence Synthetic polypeptide”
QVQLVESGGGVVQPGRSLRLSCAASGFTFS

SEQ ID NO: 42 - protein, 14 aa, Artificial Sequence
  source: /note=“Description of Artificial Sequence Synthetic peptide”
TLRSGINLGTYRIY

SEQ ID NO: 43 - protein, 32 aa, Artificial Sequence
  source: /note=“Description of Artificial Sequence Synthetic polypeptide”
RFTISRDNSKNTLYLQMNSLRAEDTAVYYCAR

SEQ ID NO: 44 - protein, 10 aa, Artificial Sequence
  source: /note=“Description of Artificial Sequence Synthetic peptide”
FGGGTKLTVL

SEQ ID NO: 45 - protein, 117 aa, Artificial Sequence
  source: /note=“Description of Artificial Sequence Synthetic polypeptide”
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRDRAFDIWGQGTMVTVSS

SEQ ID NO: 46 - protein, 119 aa, Artificial Sequence
  source: /note=“Description of Artificial Sequence Synthetic polypeptide”
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDRRGGGYGDYWGQGTLVTVSS

SEQ ID NO: 47 - protein, 30 aa, Artificial Sequence
  source: /note=“Description of Artificial Sequence Synthetic polypeptide”
EVQLVETGGGLIQPGGSLRLSCAASGFTVS

SEQ ID NO: 48 - protein, 115 aa, Artificial Sequence
  source: /note=“Description of Artificial Sequence Synthetic polypeptide”
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCARGLGSADYWGQGTLVTVSS

SEQ ID NO: 49 - protein, 23 aa, Artificial Sequence
  source: /note=“Description of Artificial Sequence Synthetic peptide”
DVVMTQSPLSLPVTLGQPASISC

SEQ ID NO: 50 - protein, 15 aa, Artificial Sequence
  source: /note=“Description of Artificial Sequence Synthetic peptide”
WFQQRPGQSPRRLIY

SEQ ID NO: 51 - protein, 117 aa, Artificial Sequence
  source: /note=“Description of Artificial Sequence Synthetic polypeptide”
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSARNWVFGGGTKLTVL

SEQ ID NO: 52 - protein, 117 aa, Artificial Sequence
  source: /note=“Description of Artificial Sequence Synthetic polypeptide”
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSARNWVFGGGTQLTVL

SEQ ID NO: 53 - protein, 14 aa, Artificial Sequence
  source: /note=“Description of Artificial Sequence Synthetic peptide”
WVRQAPGKGLEWVS

SEQ ID NO: 54 - protein, 112 aa, Artificial Sequence
  source: /note=“Description of Artificial Sequence Synthetic polypeptide”
DVVMTQSPLSLPVTLGQPASISCRSSQSLLHSNGNTYLNWFQQRPGQSPRRLIYKVSIRDSGVPDRFSGSGSGTDFTLKISRVEAEDVGLYYCMQSTQWPITFGGGTKLEIK

SEQ ID NO: 55 - protein, 120 aa, Artificial Sequence
  source: /note=“Description of Artificial Sequence Synthetic polypeptide”
QAVLTQPASLSASPGASASLTCTLRSGINVGTYRIYWYQQKPGSPPQYLLRIIKSGSSDKQQGSGVPSRFSGSKDASANAGILLISGLQSEDEADYYCMIWHSSARNWVFGGGTKLTVLG

SEQ ID NO: 56 - protein, 118 aa, Artificial Sequence
  source: /note=“Description of Artificial Sequence Synthetic polypeptide”
QAVLTQPASLSASPGASASLTCTLRSGINLGTYRIYWYQQKPGSPPQYLLRYKSDSDKQQGSGVPSRFSGSKDASANAGILLISGLQSEDEADYYCMIWHSSARNWVFGGGTKLTVLG

SEQ ID NO: 57 - DNA, 351 bp, Artificial Sequence
  source: /note=“Description of Artificial Sequence Synthetic polynucleotide”
caagtgcagctggtcgagagcggaggaggagtggtgcagcccggcagatctctgagactgagctgtgccgcctccggcttcaccttcagcagctacgccatgcactgggtgagacaagcccccggcaagggactggaatgggtggccgtcatctcctacgacggctccaacaagtactacgccgacagcgtgaagggaagattcaccatctctagagacaacagcaagaacacactgtatctgcagatgaactctctgagagctgaggacacagccgtgtactattgcgctagggtgagagatagagccttcgacatctggggccaaggcaccatggtgaccgtgagctca

SEQ ID NO: 58 - DNA, 351 bp, Artificial Sequence
  source: /note=“Description of Artificial Sequence Synthetic polynucleotide”
caagccgtgctgacacaacccgccagcctcagcgccagccccggcgctagcgcttctctgacatgcacactgaggtccggcatcaacgtgggcacctatagaatctactggtaccagcagaaacccggctcccctcctcagtatctgctgaggtacaagtccgatagcgacaagcagcaaggctccggcgtgccttctagatttagcggcagcaaggatgccagcgccaatgccggcattctgctgatcagcggactgcagagcgaggatgaggccgactactactgcatgatctggcactccagcgccagaaactgggtgttcggcggcggaaccaagctgaccgtgcta

SEQ ID NO: 59 - DNA, 357 bp, Artificial Sequence
  source: /note=“Description of Artificial Sequence Synthetic polynucleotide”
gaggtgcagctggtggaaagcggaggcggagtggtgcagcccggcagatctctgaggctgagctgtgccgctagcggcttcaccttcagcagctacggcatgcactgggtgaggcaagcccccggcaagggactggagtgggtcgccgtgatcagctacgacggcagcaacaagtactacgccgacagcgtgaagggaagattcaccatctctagagacaacagcaagaacaccctctacctccagatgaactctctgagggccgaggataccgccgtgtactactgcgccaaggacagaagaggcggcggatacggcgattactggggccaaggcacactggtgacagtgagctca

SEQ ID NO: 60 - DNA, 351 bp, Artificial Sequence
  source: /note=“Description of Artificial Sequence Synthetic polynucleotide”
caagccgtgctgacccagcccgcctctctgagcgctagccccggcgcctccgcttctctgacatgcacactgaggtccggaatcaacgtgggcacctatagaatctactggtaccagcagaagcccggcagccctcctcagtatctgctgagatacaagagcgacagcgataagcagcaaggctccggagtgcctagcagattcagcggcagcaaagacgccagcgccaatgccggaattctgctgatcagcggactgcagagcgaggacgaagccgactactactgcatgatctggcactccagcgccagaaactgggtgtttggcggcggcacccagctgacagtgcta

SEQ ID NO: 61 - DNA, 351 bp, Artificial Sequence
  source: /note=“Description of Artificial Sequence Synthetic polynucleotide”
caagtgcagctggtcgagagcggaggaggagtggtgcagcccggcagatctctgaggctgagctgcgccgccagcggattcaccttcagctcctacgccatgcactgggtgagacaagcccccggcaagggactggagtgggtggccgtgatttcctacgacggctccaacaagtactacgccgacagcgtgaagggaagattcaccatctctagagacaacagcaagaacacactgtatctgcagatgaactctctgagagccgaggacaccgccgtgtactactgcgccagagtgagggacagagccttcgacatttggggccaaggcaccatggtgacagtgagctca

SEQ ID NO: 62 - DNA, 351 bp, Artificial Sequence
  source: /note=“Description of Artificial Sequence Synthetic polynucleotide”
caagccgtgctgacccagcccgcctctctgagcgctagccccggcgctagcgcttctctgacatgcacactgaggagcggcatcaacgtgggcacctatagaatctactggtaccagcagaagcccggcagccctcctcagtatctgctgagatacaagtccgacagcgacaagcagcaaggcagcggcgtgccttctagattcagcggcagcaaggacgccagcgctaatgccggcattctgctgatcagcggactgcagagcgaggatgaggccgactactactgcatgatctggcacagcagcgccagaaactgggtgttcggcggcggcaccaagctgacagtgcta

SEQ ID NO: 63 - DNA, 345 bp, Artificial Sequence
  source: /note=“Description of Artificial Sequence Synthetic polynucleotide”
gaggtgcagctggtggaaaccggcggcggactgattcagcccggaggatctctgaggctgagctgtgccgctagcggcttcaccgtgagcagcaactatatgagctgggtgagacaagcccccggcaaaggactggagtgggtgagcgtgatctacagcggcggcagcacatactacgccgacagcgtgaagggaagattcaccatctctagagacaacagcaagaacacactgtatctgcagatgaactctctgagggccgaggacaccgccgtgtactactgcgccagaggactgggcagcgctgattactggggccaaggcacactggtgacagtgtcctca

SEQ ID NO: 64 - DNA, 336 bp, Artificial Sequence
  source: /note=“Description of Artificial Sequence Synthetic polynucleotide”
gacgtggtgatgacccagagccctctgtctctgcccgtgacactgggacagcccgccagcatcagctgcagaagctcccagtctctgctgcacagcaatggcaacacctatctgaactggttccagcaaagacccggccagtcccccagaaggctgatctacaaggtgagcattagagatagcggcgtgcccgacagatttagcggcagcggaagcggcacagacttcacactgaagatctctagagtggaggctgaggacgtgggactgtactactgcatgcagagcacccagtggcccatcacctttggcggcggcaccaagctggagatcaaa

SEQ ID NO: 65 - DNA, 351 bp, Artificial Sequence
  source: /note=“Description of Artificial Sequence Synthetic polynucleotide”
caagtgcagctggtcgagagcggaggaggagtggtgcagcccggcagatctctgaggctgagctgcgccgccagcggattcaccttcagctcctacgccatgcactgggtgagacaagcccccggcaagggactggagtgggtggccgtgatttcctacgacggctccaacaagtactacgccgacagcgtgaagggaagattcaccatctctagggacaacagcaagaacacactgtatctgcagatgaactctctgagagccgaggacaccgccgtgtactactgcgccagagtgagggacagagccttcgacatttggggccaaggcaccatggtgacagtgagctca

SEQ ID NO: 66 - DNA, 360 bp, Artificial Sequence
  source: /note=“Description of Artificial Sequence Synthetic polynucleotide”
caagccgtgctgacccagcccgcctctctgagcgctagccccggcgctagcgcctctctgacatgcacactgagaagcggcatcaacgtgggcacctatagaatctactggtaccagcagaaacccggctccccccctcagtatctgctgagaatcatcaagagcggcagcagcgacaaacagcaaggcagcggcgtgcctagcagattcagcggctccaaggatgccagcgccaatgccggcattctgctgatctccggactgcagagcgaggacgaggccgactactactgcatgatctggcacagctccgccagaaactgggtgttcggcggcggcacaaagctgacagtgctgggc

SEQ ID NO: 67 - DNA, 351 bp, Artificial Sequence; identical to SEQ ID NO: 65
  source: /note=“Description of Artificial Sequence Synthetic polynucleotide”

SEQ ID NO: 68 - DNA, 354 bp, Artificial Sequence
  source: /note=“Description of Artificial Sequence Synthetic polynucleotide”
caagccgtgctgacccagcccgcctctctgagcgctagccccggcgcttccgcctctctgacatgcacactgaggtccggcatcaatctgggcacctatagaatctactggtaccagcagaagcccggcagccctccccagtatctgctgaggtacaagagcgacagcgataagcagcaaggcagcggcgtgcctagcagatttagcggaagcaaggacgcctccgctaatgccggcattctgctgatcagcggactgcagagcgaggatgaggccgactactactgcatgatctggcactcctccgccagaaactgggtgttcggcggaggcaccaagctgacagtgctgggc

SEQ ID NO: 69 - protein, 32 aa, Artificial Sequence
  source: /note=“Description of Artificial Sequence Synthetic polypeptide”
GVPDRFSGSGSGTDFTLKISRVEAEDVGLYYC

SEQ ID NO: 70 - protein, 10 aa, Artificial Sequence
  source: /note=“Description of Artificial Sequence Synthetic peptide”
FGGGTKLEIK

SEQ ID NO: 71 - protein, 465 aa, Artificial Sequence
  source: /note=“Description of Artificial Sequence Synthetic polypeptide”
MAVLGLLLCLVTFPSCVLSQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRDRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 465 aa, Artificial Sequence
  source: /note=“Description of Artificial Sequence Synthetic polypeptide”
MAVLGLLLCLVTFPSCVLSQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRDRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 243 aa, Artificial Sequence
  source: /note=“Description of Artificial Sequence Synthetic polypeptide”
METDTLLLWVLLLWVPGSTGQAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSARNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 446 aa, Artificial Sequence
  source: /note=“Description of Artificial Sequence Synthetic polypeptide”
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRDRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 446 aa, Artificial Sequence
  source: /note=“Description of Artificial Sequence Synthetic polypeptide”
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRDRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 223 aa, Artificial Sequence
  source: /note=“Description of Artificial Sequence Synthetic polypeptide”
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSARNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - DNA, 1398 bp, Artificial Sequence
  source: /note=“Description of Artificial Sequence Synthetic polynucleotide”
atggctgtcctggggctgcttctctgcctggtgacgttcccaagctgtgtcttaagccaagtgcagctggtcgagagcggaggaggagtggtgcagcccggcagatctctgagactgagctgtgccgcctccggcttcaccttcagcagctacgccatgcactgggtgagacaagcccccggcaagggactggaatgggtggccgtcatctcctacgacggctccaacaagtactacgccgacagcgtgaagggaagattcaccatctctagagacaacagcaagaacacactgtatctgcagatgaactctctgagagctgaggacacagccgtgtactattgcgctagggtgagagatagagccttcgacatctggggccaaggcaccatggtgaccgtgagctcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgaggctctgcacaaccactacacgcagaagagcctctccctgtctccgggttaa

SEQ ID NO: 78 - DNA, 1398 bp, Artificial Sequence
  source: /note=“Description of Artificial Sequence Synthetic polynucleotide”
atggctgtcctggggctgcttctctgcctggtgacgttcccaagctgtgtcttaagccaagtgcagctggtcgagagcggaggaggagtggtgcagcccggcagatctctgagactgagctgtgccgcctccggcttcaccttcagcagctacgccatgcactgggtgagacaagcccccggcaagggactggaatgggtggccgtcatctcctacgacggctccaacaagtactacgccgacagcgtgaagggaagattcaccatctctagagacaacagcaagaacacactgtatctgcagatgaactctctgagagctgaggacacagccgtgtactattgcgctagggtgagagatagagccttcgacatctggggccaaggcaccatggtgaccgtgagctcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccggatgtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtgtccaacaaagccctcccagcccccgaggagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgaggctctgcacaaccactacacgcagaagagcctctccctgtctccgggttaa

SEQ ID NO: 79 - DNA, 732 bp, Artificial Sequence
  source: /note=“Description of Artificial Sequence Synthetic polynucleotide”
atggagacagacacactcctgctatgggtgctgctgctctgggtaccaggttccacaggtcaagccgtgctgacacaacccgccagcctcagcgccagccccggcgctagcgcttctctgacatgcacactgaggtccggcatcaacgtgggcacctatagaatctactggtaccagcagaaacccggctcccctcctcagtatctgctgaggtacaagtccgatagcgacaagcagcaaggctccggcgtgccttctagatttagcggcagcaaggatgccagcgccaatgccggcattctgctgatcagcggactgcagagcgaggatgaggccgactactactgcatgatctggcactccagcgccagaaactgggtgttcggcggcggaaccaagctgaccgtgctaggccagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggaaaccaccacaccctccaaacaaagcaacaacaagtacgcggccagcagctacctgagcctgacgcccgagcagtggaagtcccacagaagctacagctgccaggtcacgcatgaagggagcaccgtggaaaagacagtggcccctacagaatgttcataa

SEQ ID NO: 80 - DNA, 1341 bp, Artificial Sequence
  source: /note=“Description of Artificial Sequence Synthetic polynucleotide”
caagtgcagctggtcgagagcggaggaggagtggtgcagcccggcagatctctgagactgagctgtgccgcctccggcttcaccttcagcagctacgccatgcactgggtgagacaagcccccggcaagggactggaatgggtggccgtcatctcctacgacggctccaacaagtactacgccgacagcgtgaagggaagattcaccatctctagagacaacagcaagaacacactgtatctgcagatgaactctctgagagctgaggacacagccgtgtactattgcgctagggtgagagatagagccttcgacatctggggccaaggcaccatggtgaccgtgagctcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgaggctctgcacaaccactacacgcagaagagcctctccctgtctccgggttaa

SEQ ID NO: 81 - DNA, 1341 bp, Artificial Sequence
  source: /note=“Description of Artificial Sequence Synthetic polynucleotide”
caagtgcagctggtcgagagcggaggaggagtggtgcagcccggcagatctctgagactgagctgtgccgcctccggcttcaccttcagcagctacgccatgcactgggtgagacaagcccccggcaagggactggaatgggtggccgtcatctcctacgacggctccaacaagtactacgccgacagcgtgaagggaagattcaccatctctagagacaacagcaagaacacactgtatctgcagatgaactctctgagagctgaggacacagccgtgtactattgcgctagggtgagagatagagccttcgacatctggggccaaggcaccatggtgaccgtgagctcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccggatgtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtgtccaacaaagccctcccagcccccgaggagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgaggctctgcacaaccactacacgcagaagagcctctccctgtctccgggttaa

SEQ ID NO: 82 - DNA, 672 bp, Artificial Sequence
  source: /note=“Description of Artificial Sequence Synthetic polynucleotide”
caagccgtgctgacacaacccgccagcctcagcgccagccccggcgctagcgcttctctgacatgcacactgaggtccggcatcaacgtgggcacctatagaatctactggtaccagcagaaacccggctcccctcctcagtatctgctgaggtacaagtccgatagcgacaagcagcaaggctccggcgtgccttctagatttagcggcagcaaggatgccagcgccaatgccggcattctgctgatcagcggactgcagagcgaggatgaggccgactactactgcatgatctggcactccagcgccagaaactgggtgttcggcggcggaaccaagctgaccgtgctaggccagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggaaaccaccacaccctccaaacaaagcaacaacaagtacgcggccagcagctacctgagcctgacgcccgagcagtggaagtcccacagaagctacagctgccaggtcacgcatgaagggagcaccgtggaaaagacagtggcccctacagaatgttcataa

(19) United States
(12) Patent Application Publication
(10) Pub. No.: US 2023/0416382 A1
(43) Pub. Date: Dec. 28, 2023

(54) ANTI-C-C CHEMOKINE RECEPTOR 8 (CCR8) ANTIBODIES AND METHODS OF USE THEREOF

(71) Applicant: Five Prime Therapeutics, Inc., Thousand Oaks, CA (US)

(72) Inventors: Susannah D. BARBEE, San Francisco, CA (US); Shujun YUAN, San Mateo, CA (US); Marcel MEURY, Foster City, CA (US); Alessandro PALUMBO, San Francisco, CA (US); Terence WONG, Alameda, CA (US); Kathrin ZUBERBUHLER, Pacifica, CA (US); Emilia FALKOWSKA, San Francisco, CA (US)

(73) Assignee: Five Prime Therapeutics, Inc., Thousand Oaks, CA (US)

(21) Appl. No.: 18/248,979

(22) PCT Filed: Oct. 13, 2021
(86) PCT No.: PCT/US2021/054797

§ 371 Date: Apr. 13, 2023

Related U.S. Application Data
(60) Provisional application No. 63/107,755, filed on Oct. 30, 2020.
(60) Provisional application No. 63/091,889, filed on Oct. 14, 2020.

(51) Int. Cl. C07K 16/28; A61P 35/00

(52) U.S. Cl. CPC C07K 16/2866; A61P 35/00; C07K 2317/565

(57) ABSTRACT

This present invention provides anti-C—C chemokine type 8 (CCR8) antibodies and antigen binding fragments thereof, methods of making the antibodies or antigen binding fragments thereof, and methods of use thereof to bind to human CCR8 on CCR8 expressing cells, e.g., tumor-infiltrating Treg cells, to remove CCR8 expressing cells, e.g, tumor-infiltrating Treg cells, to reduce or inhibit tumor growth and/or to treat cancer.

### RELATED APPLICATIONS

This application claims the benefit of priority to U.S. Provisional Application No. 63/091,889, filed on Oct. 14, 2020, and U.S. Provisional Application No. 63/107,755, filed on Oct. 30, 2020. The entire contents of each of these applications are incorporated herein by reference.

### BACKGROUND

Regulatory T (Treg) cells are a major immune cell population that plays a crucial role in maintaining self-tolerance and resolution of immune responses by employing multifaceted immunoregulatory mechanisms (Vignali D A, et al., Nat Rev Immunol 2008; 8:523-32). However, Treg cells readily infiltrate into the tumor microenvironment (TME) and dampen anti-tumor immune responses, thereby becoming a barrier to effective cancer immunotherapy (Tanaka A, Sakaguchi S. Cell Res 2017; 27:109-18). Treg modulation strategies have been shown to increase antitumor immunity and reduce tumor burden in both preclinical and clinical settings (Gooden M J, et al., Br J Cancer 2011; 105:93-103). However, although these strategies have demonstrated enhanced antitumor immune responses, certain drawbacks exist, such as autoimmunity and specificity of targeting (Curtin J F M, et al., PLoS One 2008; 3:e1983). Because Tregs and activated effector lymphocytes both express surface molecules that can be used as therapeutic targets, there is the potential for ablation of essential tumor-specific effector cells required to control tumor progression in these types of antibody-mediated immunotherapies (Nishikawa H, Sakaguchi S. Int J Cancer 2010; 127:759-67). Therefore, the development of a more effective approach to specifically and selectively target tumor-infiltrating Tregs is required.

C—C chemokine receptor 8 (CCR8) is a chemokine receptor that is selectively expressed on a subset of intratumoral Tregs bearing the highest levels of suppressive markers, and its expression correlates with poor prognosis in multiple tumor types (Yano H, et al., Immunology 2019, 157: 232-247). This subset of Tregs expressing CCR8 (CCR8+ Tregs) has been demonstrated to be a major driver of immunosuppression and is critical for Treg function and suppression (Barsheshet Y, et al., Proc Natl Acad Sci USA 2017; 114:6086-91). However, there are currently no known CCR8-targeted therapeutics in clinical trials.

Accordingly, there remains a need in the art to develop CCR8-targeted therapeutics, such as anti-CCR8 antibodies, that can be used for therapeutic purposes in the treatment of cancer.

### SUMMARY OF THE INVENTION

The present invention provides anti-C—C chemokine type 8 (CCR8) antibodies and antigen binding fragments thereof, methods of making the antibodies or antigen binding fragments thereof, and methods of using such antibodies to detect human CCR8, to bind to human CCR8 on CCR8 expressing cells, e.g., tumor-infiltrating Treg cells, to remove CCR8 expressing cells, e.g, tumor-infiltrating Treg cells, to reduce or inhibit tumor growth and/or to treat cancer.

Accordingly, the present invention provides, in one aspect, an antibody, or antigen-binding fragment thereof, that binds to C—C Chemokine Receptor 8 (CCR8), wherein the antibody, or antigen-binding fragment thereof, has an enhanced antibody-dependent cell-mediated cytotoxicity (ADCC) activity.

In another aspect, the present invention provides an antibody, or antigen-binding fragment thereof, that binds to C—C Chemokine Receptor 8 (CCR8), wherein the antibody, or antigen-binding fragment thereof, has a dissociation constant (K_{d}) for CCR8 less than 10 nM.

In one aspect, the present invention provides an antibody, or antigen-binding fragment thereof, that binds to C—C Chemokine Receptor 8 (CCR8), wherein the antibody, or antigen-binding fragment thereof, induces Fc receptor activation with an EC50 less than 3 nM.

In another aspect, the present invention provides an antibody, or antigen-binding fragment thereof, that binds to C—C Chemokine Receptor 8 (CCR8), wherein the antibody, or antigen-binding fragment thereof, induces natural killer cell-mediated killing against CCR8 expressing cells with an EC50 less than 1 nM.

In some embodiments, the antibody, or antigen-binding fragment thereof, specifically binds to human CCR8 and/or Cynomolgus CCR8. In some embodiments, the antibody, or antigen-binding fragment thereof, does not bind to murine CCR8.

In some embodiments, the CCR8 expressing cells comprise tumor infiltrating regulatory T (Treg) cells.

In some embodiments, the antibody, or antigen-binding fragment thereof, comprises an isotype selected from a group consisting of hIgG1, hIgG2, hIgG3, hIgG4, mIgG1 and mIgG2a. In some embodiments, the antibody, or antigen-binding fragment thereof, comprises an isotype of hIgG1.

In some embodiments, the antibody, or antigen-binding fragment thereof, is a humanized antibody or antigen-binding fragment thereof.

In some embodiments, the antibody, or antigen-binding fragment thereof, has a mutated Fc region. In other embodiments, the antibody, or antigen-binding fragment thereof, comprises one or more mutations selected from a group consisting of S239D, A330L and I332E. In another embodiment, the antibody, or antigen-binding fragment thereof, comprises each of the mutations S239D, A330L and I332E.

In some embodiments, the antibody, or antigen-binding fragment thereof, has an enhanced ADCC activity against CCR8-expressing cells. In other embodiments, CCR8-expressing cells are tumor-infiltrating regulatory T (Treg) cells.

In some embodiments, the antibody, or antigen-binding fragment thereof, binds and/or removes tumor-infiltrating Treg cells. In other embodiments, the antibody, or antigen-binding fragment thereof, has no effect on peripheral Treg cells.

In some embodiments, the antibody, or antigen-binding fragment thereof, is not internalized by an effector cell. In some embodiments, the effector cell is selected from a group consisting of natural killer (NK) cells, macrophages, neutrophils and eosinophils.

In some embodiments, the antibody, or antigen-binding fragment thereof, elicits an antigen-specific memory response.

In some embodiments, the antibody, or antigen-binding fragment thereof, comprises a heavy chain variable region comprising a CDR3 domain comprising an amino acid sequence selected from a group consisting of SEQ ID NOs: 11, 17, 23, and 29.

In some embodiments, the antibody, or antigen-binding fragment thereof, comprises a heavy chain variable region comprising a CDR2 domain comprising an amino acid sequence selected from a group consisting of SEQ ID NOs: 10, 16, and 28.

In some embodiments, the antibody, or antigen-binding fragment thereof, comprises a heavy chain variable region comprising a CDR1 domain comprising an amino acid sequence selected from a group consisting of SEQ ID NOs: 9, 15, 21, and 27.

In some embodiments, the antibody, or antigen-binding fragment thereof, comprises a light chain variable region comprising a CDR3 domain comprising an amino acid sequence selected from a group consisting of SEQ ID NOs: 14, 20, and 32.

In some embodiments, the antibody, or antigen-binding fragment thereof, comprises a light chain variable region comprising a CDR2 domain comprising an amino acid sequence selected from a group consisting of SEQ ID NOs: 13, 31, and 37.

In some embodiments, the antibody, or antigen-binding fragment thereof, comprises a light chain variable region comprising a CDR1 domain comprising an amino acid sequence selected from a group consisting of SEQ ID NOs: 12, 30, and 42.

In some embodiments, the antibody, or antigen-binding fragment thereof, comprises a heavy chain variable region comprising an amino acid sequence selected from a group consisting of SEQ ID NOs: 45, 46 and 48.

In some embodiments, the antibody, or antigen-binding fragment thereof, comprises a light chain variable region comprising an amino acid sequence selected from a group consisting of SEQ ID NOs: 51, 52 and 54-56.

In some embodiments, the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO:10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:14.

In some embodiments, the antibody, or antigen-binding fragment thereof, comprises a heavy chain variable region having a CDR1 domain comprising the amino acid sequence of SEQ ID NO: 15, a CDR2 domain comprising the amino acid sequence set forth in SEQ ID NO:16; and a CDR3 domain comprising the amino acid sequence set forth in SEQ ID NO:17; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:20.

In some embodiments, the antibody, or antigen-binding fragment thereof, comprises a heavy chain variable region having a CDR1 domain comprising the amino acid sequence of SEQ ID NO: 21, a CDR2 domain comprising the amino acid sequence set forth in SEQ ID NO:16; and a CDR3 domain comprising the amino acid sequence set forth in SEQ ID NO:23; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:20.

In some embodiments, the antibody, or antigen-binding fragment thereof, comprises a heavy chain variable region having a CDR1 domain comprising the amino acid sequence of SEQ ID NO: 27, a CDR2 domain comprising the amino acid sequence set forth in SEQ ID NO:28; and a CDR3 domain comprising the amino acid sequence set forth in SEQ ID NO:29; and a light chain variable region having a CDR1 domain comprising the amino acid sequence set forth in SEQ ID NO:30; a CDR2 domain comprising the amino acid sequence set forth in SEQ ID NO:31; and a CDR3 domain comprising the amino acid sequence set forth in SEQ ID NO:32.

In some embodiments, the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:37; and a CDR3 domain comprising the amino acid sequence set forth in SEQ ID NO:14.

In some embodiments, the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:42; a CDR2 domain comprising the amino acid sequence set forth in SEQ ID NO:13; and a CDR3 domain comprising the amino acid sequence set forth in SEQ ID NO:14.

In some embodiments, the antibody, or antigen-binding fragment thereof, comprises a heavy chain variable region comprising the amino acid sequence of SEQ ID NO: 45, and a light chain variable region comprising the amino acid sequence set forth in SEQ ID NO:51.

In some embodiments, the antibody, or antigen-binding fragment thereof, comprises a heavy chain variable region comprising the amino acid sequence of SEQ ID NO:46, and a light chain variable region comprising the amino acid sequence set forth in SEQ ID NO:52.

In some embodiments, the antibody, or antigen-binding fragment thereof, comprises a heavy chain variable region comprising the amino acid sequence of SEQ ID NO:48, and a light chain variable region comprising the amino acid sequence set forth in SEQ ID NO:54.

In some embodiments, the antibody, or antigen-binding fragment thereof, comprises a heavy chain variable region comprising the amino acid sequence of SEQ ID NO:45, and a light chain variable region comprising the amino acid sequence set forth in SEQ ID NO:55.

In some embodiments, the antibody, or antigen-binding fragment thereof, comprises a heavy chain variable region comprising the amino acid sequence of SEQ ID NO:45, and a light chain variable region comprising the amino acid sequence set forth in SEQ ID NO:56.

In some embodiments, the antibody, or antigen-binding fragment thereof, comprises a heavy chain comprising the amino acid sequence of SEQ ID NO:74, and a light chain comprising the amino acid sequence set forth in SEQ ID NO:76.

In some embodiments, the antibody, or antigen-binding fragment thereof, comprises a heavy chain comprising the amino acid sequence of SEQ ID NO:75, and a light chain comprising the amino acid sequence set forth in SEQ ID NO:76.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 9, a CDR2 domain comprising the amino acid sequence set forth in SEQ ID NO:10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:14.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 15, a CDR2 domain comprising the amino acid sequence set forth in SEQ ID NO:16; and a CDR3 domain comprising the amino acid sequence set forth in SEQ ID NO:17; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth 16 in SEQ ID NO:20.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 21, a CDR2 domain comprising the amino acid sequence set forth in SEQ ID NO:16; and a CDR3 domain comprising the amino acid sequence set forth in SEQ ID NO:23; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:20.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 27, a CDR2 domain comprising the amino acid sequence set forth in SEQ ID NO:28; and a CDR3 domain comprising the amino acid sequence set forth in SEQ ID NO:29; and a light chain variable region having a CDR1 domain comprising the amino acid sequence set forth in SEQ ID NO:30; a CDR2 domain comprising the amino acid sequence set forth in SEQ ID NO:31; and a CDR3 domain comprising the amino acid sequence set forth in SEQ ID NO:32.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:37; and a CDR3 domain comprising the amino acid sequence set forth in SEQ ID NO:14.

In one aspect, the present invention provides an antibody, or antigen-binding fragment thereof, that binds to C—C Chemokine Receptor 8 (CCR8) comprising a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:42; a CDR2 domain comprising the amino acid sequence set forth in SEQ ID NO:13; and a CDR3 domain comprising the amino acid sequence set forth in SEQ ID NO:14.

In one aspect, the present invention provides an antibody, or antigen-binding fragment thereof, that binds to C—C Chemokine Receptor 8 (CCR8) comprising a heavy chain variable region comprising the amino acid sequence of SEQ ID NO: 45, and a light chain variable region comprising the amino acid sequence set forth in SEQ ID NO:51.

In one aspect, the present invention provides an antibody, or antigen-binding fragment thereof, that binds to C—C Chemokine Receptor 8 (CCR8) comprising a heavy chain variable region comprising the amino acid sequence of SEQ ID NO:46, and a light chain variable region comprising the amino acid sequence set forth in SEQ ID NO:52.

In one aspect, the present invention provides an antibody, or antigen-binding fragment thereof, that binds to C—C Chemokine Receptor 8 (CCR8) comprising a heavy chain variable region comprising the amino acid sequence of SEQ ID NO:48, and a light chain variable region comprising the amino acid sequence set forth in SEQ ID NO:54.

In one aspect, the present invention provides an antibody, or antigen-binding fragment thereof, that binds to C—C Chemokine Receptor 8 (CCR8) comprising a heavy chain variable region comprising the amino acid sequence of SEQ ID NO:45, and a light chain variable region comprising the amino acid sequence set forth in SEQ ID NO:55.

In one aspect, the present invention provides an antibody, or antigen-binding fragment thereof, that binds to C—C Chemokine Receptor 8 (CCR8) comprising a heavy chain variable region comprising the amino acid sequence of SEQ ID NO:45, and a light chain variable region comprising the amino acid sequence set forth in SEQ ID NO:56.

In one aspect, the present invention provides an antibody, or antigen-binding fragment thereof, that binds to C—C Chemokine Receptor 8 (CCR8) comprising a heavy chain comprising the amino acid sequence of SEQ ID NO:74, and a light chain comprising the amino acid sequence set forth in SEQ ID NO:76.

In one aspect, the present invention provides an antibody, or antigen-binding fragment thereof, that binds to C—C Chemokine Receptor 8 (CCR8) comprising a heavy chain comprising the amino acid sequence of SEQ ID NO:75, and a light chain comprising the amino acid sequence set forth in SEQ ID NO:76.

In one aspect, the present invention provides an antibody which competes for binding to CCR8 with an antibody, or antigen binding fragment thereof, as described herein.

In one aspect, the present invention provides an antibody which binds to the same epitope on CCR8 as an antibody, or antigen binding fragment thereof, as described herein.

In one aspect, the present invention provides a pharmaceutical composition comprising an antibody, or antigen-binding fragment thereof, as described herein, and a pharmaceutically acceptable carrier.

In one aspect, the present invention provides a kit comprising an antibody, or antigen-binding fragment thereof, as described herein, or a pharmaceutical composition of the invention and instructions for use.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 51 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12-14 respectively, and wherein the VL when paired with a heavy chain variable region (VH) comprising the amino acid sequence set forth in SEQ ID NO: 45 binds to CCR8.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 15-17, respectively, and wherein the VH when paired with a light chain variable region (VL) comprising the amino acid sequence set forth in SEQ ID NO: 52 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 13 and 20, respectively, and wherein the VL when paired with a heavy chain variable region (VH) comprising the amino acid sequence set forth in SEQ ID NO: 46 binds to CCR8.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 21, 16 and 23, respectively, and wherein the VH when paired with a light chain variable region (VL) comprising the amino acid sequence set forth in SEQ ID NO: 51 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 13 and 20 respectively, and wherein the VL when paired with a heavy chain variable region (VH) comprising the amino acid sequence set forth in SEQ ID NO: 45 binds to CCR8.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 27-29, respectively, and wherein the VH when paired with a light chain variable region (VL) comprising the amino acid sequence set forth in SEQ ID NO: 54 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 30-32, respectively, and wherein the VL when paired with a heavy chain variable region (VH) comprising the amino acid sequence set forth in SEQ ID NO: 48 binds to CCR8.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 55 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 37 and 14, respectively, and wherein the VL when paired with a heavy chain variable region (VH) comprising the amino acid sequence set forth in SEQ ID NO: 45 binds to CCR8.

In one aspect, the present invention provides a polynucleotide comprising a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 56 binds to CCR8.

In one aspect, the present invention provides a polynucleotide comprising a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 42, 13 and 14, respectively, and wherein the VL when paired with a heavy chain variable region (VH) comprising the amino acid sequence set forth in SEQ ID NO: 45 binds to CCR8.

In one aspect, the present invention provides a polynucleotide comprising a polynucleotide encoding a polypeptide comprising an immunoglobulin light chain comprising the amino acid sequence set forth in SEQ ID NO: 76, and wherein the light chain when paired with a heavy chain comprising the amino acid sequence set forth in SEQ ID NO: 74 binds to CCR8.

In one aspect, the present invention provides a polynucleotide comprising a polynucleotide encoding a polypeptide comprising an immunoglobulin light chain comprising the amino acid sequence set forth in SEQ ID NO: 76, and wherein the light chain when paired with a heavy chain comprising the amino acid sequence set forth in SEQ ID NO: binds to CCR8.

In one aspect, the present invention provides a polynucleotide comprising a polynucleotide encoding a polypeptide comprising an immunoglobulin heavy chain comprising the amino acid sequence set forth in SEQ ID NO: 74, and wherein the heavy chain when paired with a light chain comprising the amino acid sequence set forth in SEQ ID NO: 76 binds to CCR8.

In one aspect, the present invention provides a polynucleotide comprising a polynucleotide encoding a polypeptide comprising an immunoglobulin heavy chain comprising the amino acid sequence set forth in SEQ ID NO: 75, and wherein the heavy chain when paired with a light chain comprising the amino acid sequence set forth in SEQ ID NO: 76 binds to CCR8.

In some embodiments, the VH when paired with a VL specifically binds to human CCR8 and/or Cynomolgus CCR8, and the VL when paired with a VH specifically binds to human CCR8 and/or Cynomolgus CCR8.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In one aspect, the present invention provides a vector comprising the polynucleotide of the invention.

In another aspect, the present invention provides a host cell comprising the polynucleotide as described herein or the vector of the invention.

In one aspect, the present invention provides a method of producing an antibody, or antigen-binding fragment thereof of, comprising expressing the antibody, or antigen-binding fragment thereof in the host cell and isolating the expressed antibody, or antigen-binding fragment thereof.

In one aspect, the present invention provides a method of treating cancer in a subject, the method comprising administering to the subject a therapeutically effective amount of the antibody or antigen-binding fragment thereof or the pharmaceutical composition as described herein, thereby treating cancer in the subject.

In some embodiments, the cancer is selected from a group consisting of colon cancer, breast cancer, lung cancer, liver cancer, pancreatic cancer, ovarian cancer, kidney cancer, bladder cancer, colorectal cancer, endometrial cancer, melanoma, squamous cell carcinoma of the head and neck, renal cell carcinoma, hepatocellular carcinoma and malignant glioma.

In some embodiments, the antibody or antigen-binding fragment thereof binds to CCR8 expressed on tumor infiltrating Treg cells, and/or removes the tumor infiltrating Treg cells in the subject.

In one aspect, the present invention provides a method of removing tumor infiltrating regulatory T (Treg) cells from a subject, the method comprising administering to the subject a therapeutically effective amount of the antibody or antigen-binding fragment thereof or the pharmaceutical composition of the invention, thereby removing tumor infiltrating Treg cells from the subject.

In one aspect, the present invention provides a method of reducing tumor growth in a subject, the method comprising administering to the subject a therapeutically effective amount of the antibody or antigen-binding fragment thereof or the pharmaceutical composition of the invention, thereby reducing tumor growth in the subject.

In one aspect, the present invention provides a method of generating an antibody or antigen-binding fragment thereof that binds specifically to human CCR8, the method comprising preparing a soluble CCR8 by presenting the CCR8 protein in a synthetic membrane system; wherein the CCR8 is a mutant form of CCR8, and generating antibodies or antigen-binding fragment thereof against the soluble CCR8.

In some embodiments, the CCR8 protein comprises one or more mutations in the intracellular region and/or the transmembrane domain.

In other embodiments, the synthetic membrane system comprises a nanodisc composed of a phospholipid bilayer encircled by two copies of a membrane scaffold protein.

### BRIEF DESCRIPTION OF THE DRAWINGS

FIGS. 1A and 1B are graphs depicting enrichment of CCR8 expression in the tumor microenvironment. Human peripheral blood mononuclear cells (PBMCs) (A) and dissociated renal cell carcinoma (RCC) specimens (B) were stained with a combination of antibodies to define leukocyte subsets and anti-CCR8 and anti-CCR4 antibodies to evaluate the expression of CCR8 and the closely related family member CCR4. Data represent mean+/−SEM of ≥4 independent samples. The following cell surface immunophenotypes were used to define naïve CD4+ effector (CD4+CD25− CTLA4−), activated CD4+ effector (CD4+CD25+ FOXP3− CTLA4−) and regulatory T cells (CD4+CD25+ FOXP3+ CTLA4− or CD4+CD25+ CTLA4+). Statistical significance was determined using unpaired Student's t-test and p values less than 0.05 considered significant. ***p<0.0001, **p=0.0005 and *p=0.08.

FIGS. 2A-C are graphs depicting that an anti-murine CCR8 depleting antibody inhibits tumor growth and increases survival of CT26-tumor-bearing mice as a single agent. BALB/c mice were inoculated subcutaneously with CT26 colon carcinoma cells and treatment was initiated at an average tumor volume of 144 mm3. The anti-CCR8 mIgG2a and mIgG2a isotype control antibodies were dosed at 10 mg/kg on Days 4 and 7, whereas the anti-CTLA4 (clone 9D9) was dosed at 1 mg/kg on Days 4, 7 and 11. The study was terminated on Day 35. Tumor volumes were monitored over time (A) or assessed on Day 20 post-tumor cell inoculation (B). (C) Kaplan-Meier survival analysis. Error bars are small where not visible. Data represent mean+/−standard deviation (SD) of n=10 mice per group. Statistical significance was determined vs mIgG2a isotype control using One-way ANOVA and p<0.05 was considered significant. eADCC; Enhanced antibody dependent cellular cytotoxicity.

FIGS. 3A-C are graphs depicting that an anti-murine CCR8 depleting antibody inhibits tumor growth and increases survival of MC38-tumor-bearing mice as a single agent. C57BL/6 mice were inoculated subcutaneously with MC38 colon carcinoma cells and treatment initiated at an average tumor volume of 123 mm3. The anti-CCR8 mIgG2a and mIgG2a isotype control antibodies were dosed at 10 mg/kg on Days 6 and 9, whereas the anti-CTLA4 (clone 9D9) antibody was dosed at 1 mg/kg on Days 6, 9 and 13. The study was terminated on Day 63. Tumor volumes were monitored over time (A) or assessed on Day 23 post-tumor cell inoculation (B). (C) Kaplan-Meier survival analysis. Error bars are small where not visible. Data represent mean+/−standard deviation (SD) of n=10 mice per group. Statistical significance was determined vs mIgG2a isotype control using One-way ANOVA for (B) or Log-rank test for (C). ***p≤0.0001. eADCC; Enhanced antibody dependent cellular cytotoxicity.

FIGS. 4A-C are graphs depicting sustained depletion of intratumoral Tregs by treatment with an anti-CCR8 depleting antibody. C57BL/6 mice were inoculated subcutaneously with MC38 colon carcinoma cells and treatment initiated at an average tumor volume of 100 mm3. Mice were harvested on Days 3, 7 and 10 following a single dose of 10 mg/kg and single cell tumor suspensions generated for FACS analysis. Live CD45+ singlets were gated and CD4+ Tregs defined as CD3+CD4+ FOXP3+, CD4+ effector T cells as CD3+CD4+CD25− FOXP3− and CD8+ effector T cells as CD3+CD8+. Statistical significance was determined by Two-way ANOVA and a p value<0.05 considered significant. ***p≤0.0001

FIGS. 5A-C are graphs depicting selective depletion of murine intratumoral Tregs by treatment with an anti-CCR8 depleting antibody. C57BL/6 mice were inoculated subcutaneously with 0.5×106 MC38 colon carcinoma cells and treatment initiated at an average tumor volume of 96 mm3. Mice were harvested on Day 3 following a single dose of 3 mg/kg and single cell tumor, spleen and peripheral blood suspensions generated for FACS analysis. (A) Tumor (B) Spleen (C) Peripheral blood. Live CD45+ singlets were gated and CD4+ Tregs defined as CD3+CD4+CD25+ FOXP3+, CD4+ effector T cells as CD3+CD4+CD25− FOXP3− and CD8+ effector T cells as CD3+CD8+. Data represent mean+/−SEM of independent mice per group. Statistical significance was determined by unpaired Student's t-test and p values less then 0.05 considered significant. ****p<0.0001 and ***p=0.0002.

FIG. 6 is a graph depicting that treatment with an anti-murine CCR8 depleting antibody promotes the development of an antigen-specific memory response. CT26-tumor-bearing mice treated with anti-CCR8 mIgG2a (eADCC) antibody exhibited complete regressions. Approximately 12 weeks after the initial tumor cell inoculation, mice were re-challenged with CT26 or the unrelated tumor EMT6. Naïve mice were not previously inoculated with tumor cells. The study was terminated on Day 20 post-challenge. Statistical significance was determined by unpaired Student's t-test and p values less than 0.05 considered significant. ***p<0.0001. SEM; standard error of the mean

FIGS. 7A and 7B are graphs depicting efficacy of an anti-murine CCR8 depleting antibody in MC38 tumor-bearing humanized FcgR mice. Humanized FcgR mice were inoculated with 0.5×106 cells and treatment initiated when the average tumor volume was approximately 100 mm3. Mice were treated with a single dose of either 3 or 0.3 mg/kg of mCCR8_hIgG1 (Wild-type) or (eADCC), 3 mg/kg of the hIgG1 isotype and 5 mg/kg of the anti-PD1 control antibodies. Tumor volumes were monitored over time (A) or assessed on Day 20 post-tumor cell inoculation (B). Data represent mean+/−SEM. **p=0.001 and *** p=0.0007. Statistical significance was determined by unpaired Student's t-test and p values less then 0.05 considered significant.

FIGS. 8A-8C depict example plots from FACS analyses of binding of anti-CCR8 antibody clones to HEK 293 cells expressing either human CCR8 (A), cynomologous CCR8 (B) or murine CCR8 (C).

FIG. 9A depicts FACs analysis of huCCR8, huCCR4 and huCX3CR1 293 cells validating expression of the transfected constructs in each of the cell lines. FIG. 9B depicts an example plot from FACS analyses of binding of anti-CCR8 antibody clones to HEK 293 cells expressing either huCCR8, huCCR4 or huCX3CR1.

FIG. 10 depicts an example plot of luminescence induced in ADCC reporter cells following FcR engagement with anti-CCR8 antibodies binding Hut78 cells expressing CCR8.

FIGS. 11A and 11B are graphs depicting that an anti-human CCR8 antibody enhances the ADCC activity of primary human NK cells. (A) Assessment of CCR8 expression on the TALL1 cell line and a CCR8 KO cell line as an indicator of background signal. CCR8 receptor levels (antibody binding units) were quantified on the TALL1 cell line using Quantum Simply Cellular anti-Rat IgG microspheres (BANGs Laboratories). (B) Primary human NK cells were purified from healthy donor PBMCs and co-cultured with TALL1 cells at a target to effector ratio of 1:3. Target cell death was determined by flow cytometry four hours after assay initiation. Data represent mean+/−SEM of 3-8 independent donors. Ab; antibody, KO; knock-out.

### DETAILED DESCRIPTION OF THE INVENTION

The present invention provides anti-C—C chemokine type 8 (CCR8) antibodies and antigen binding fragments thereof, methods of making the antibodies or antigen binding fragments thereof, and methods of using such antibodies to, for example, detect human CCR8, to bind to human CCR8 on CCR8 expressing cells, e.g., tumor-infiltrating Treg cells, to remove CCR8 expressing cells, e.g, tumor-infiltrating Treg cells, to reduce or inhibit tumor growth and/or to treat cancer.

Chemokine receptors have traditionally been very difficult antigens against which to develop antibodies. They have low profiles on the cell surface and are not very accessible to antibody binding. In addition, antibodies generated against peptides corresponding to extracellular domains of chemokine receptors often fail to recognize the intact receptor on the cell, probably because of differences in secondary structure. (See, e.g., Wu et al., J. Exp. Med. 185:1681-91 (1997). Specifically, CCR8 protein has proved to be a particularly unstable protein in comparison to other multi-span G-protein coupled receptors (GPCRs). In addition, the minimal surface exposure and flexible topology makes CCR8 a challenging antibody target. Currently, no soluble protein for immunizations, sorting or screening is available. Therefore, due to these difficulties, researchers in this field have had a low success rate in developing anti-CCR8 antibodies.

The present inventors, however, have successfully developed a unique and superior approach for generating antibodies targeting the specific chemokine receptor CCR8. Specifically, the inventors developed a CCR8 mutagenesis screen in which each residue in the transmembrane and the intracellular regions of CCR8 were substituted with all 19 non-wild type amino acids in order to identify stabilizing CCR8 mutants. Subsequently, the identified CCR8 mutant is presented in a nanodisc as a soluble antigen, and used as an immunogen for antibody production. Using this approach, the inventors have successfully identified a number of anti-CCR8 antibodies as disclosed in the Examples section below.

Accordingly, the present invention provides highly specific anti-human CCR8 antibodies that do not bind the closely related chemokine receptors such as CCR4 and CX3CR1. The anti-CCR8 antibodies were engineered to enhance antibody dependent cellular cytotoxicity (ADCC) activity and elicited potent natural killer (NK) cell-mediated killing of target cells expressing CCR8 at levels observed on human intratumoral Tregs. Furthermore, the inventors have demonstrated in multiple murine tumor models that treatment of the animals with the anti-CCR8 antibodies of the present invention reduced tumor growth in a dose- and FcR-dependent manner, indicating that these antibodies are useful for treating cancer.

Without wishing to be bound by any particular theory, it is believed that the engineered antibodies with enhanced ADCC activity of the present invention possess additional advantages over other CCR8 antibodies existing in the art in that it is believed that they do not bind the ligand binding domain of CCR8 and, as a result, are not internalized by cells, e.g., effector cells of the immune system, thereby exhibiting a more effective and sustained action, e.g., depletion of tumor-infiltrating Treg cells expressing CCR8 and inhibition of tumor growth.

Accordingly, the present invention provides antibodies or antigen binding fragments thereof that specifically bind to CCR8, e.g., human CCR8. The present invention also provides methods of making the anti-CCR8 antibodies as described herein. Furthermore, the present invention provides methods of using the anti-CCR8 antibodies described herein, e.g., methods for treating or preventing cancer, methods for reducing tumor-infiltrating Treg cells, and methods for reducing or inhibiting tumor growth or tumor size, in a subject using anti-CCR8 antibodies or antigen binding fragments thereof.

The section headings used herein are for organizational purposes only and are not to be construed as limiting the subject matter described. All references cited herein, including patent applications and publications, are incorporated herein by reference in their entireties for any purpose.

### I. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, exemplary techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the present invention, which can be had by reference to the specification as a whole. Accordingly, the terms defined immediately below are more fully defined by reference to the specification in its entirety.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.”

The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means +10%. In certain embodiments, about means +5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range. As used herein, ranges include both the upper and lower limit.

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein that includes modifications, such as deletions, additions, and substitutions (generally conservative in nature), to a native sequence, as long as the protein maintains the desired activity. These modifications may be deliberate, as through site-directed mutagenesis, or may be accidental, such as through mutations of hosts that produce the proteins or errors due to PCR amplification.

As used herein, whether a particular amino acid sequence is, for example, at least 95% identical to a specific reference sequence can be determined using, e.g., a computer program. When determining whether a particular sequence is, for example, 95% identical to a reference sequence, the percentage of identity is calculated over the full length of the reference amino acid sequence.

As used herein, the term “CCR8” or “C—C Motif Chemokine Receptor 8” refers to a member of the β-chemokine receptor family, which is predicted to be a seven transmembrane protein similar to G protein-coupled receptors. CCR8 is also known as TER1, CHEMR, CMKBRL2, GPR-CY6, CDw198, CMKBR8, CKR-L1, and CY6. CCR8 is predominantly expressed on regulatory T cells (Treg) and on a small portion of Th2 cells.

The term “CCR8” includes human CCR8, the amino acid sequence of which may be found in for example, GenBank Accession No. NP_005192.1 (SEQ ID NO:1); Macaca fascicularis CCR8, the amino acid sequence of which may be found in for example, GenBank Accession No. NP_001274549.1 (SEQ ID NO:3); mouse (Mus musculus) CCR8, the amino acid sequence of which may be found in for example, GenBank Accession No. NP_031746.1 (SEQ ID NO:5); and rat (Rattus norvegicus) CCR8, the amino acid sequence of which may be found in for example, for example GenBank Accession No. XP_008764924.1 (SEQ ID NO:7).

The term “CCR8” includes a wild type, a variant or an isoform of CCR8 protein or a fragment or domain thereof. In some embodiments, the variant forms of CCR8 include those CCR8 mutants with one or more substitutions in the transmembrane or intracellular regions of the protein. These mutants are generated, for example, to enhance protein stability, while maintaining the natural binding capabilities of CCR8. The term “CCR8” also encompasses CCR8 protein or a fragment thereof coupled to, for example, a mouse or human Fc, a signal peptide sequence, and/or a protein tag.

The nucleotide sequence of human CCR8 can be found in for example, GenBank Accession No. NM_005201.4 (SEQ ID NO: 2). The nucleotide sequence of Macaca fascicularis CCR8 can be found in for example, GenBank Accession No. NM_001287620.1 (SEQ ID NO: 4). The nucleotide sequence of mouse CCR8 can be found in for example, GenBank Accession No. NM_007720.2 (SEQ ID NO: 6). The nucleotide sequence of rat CCR8 can be found in for example, GenBank Accession No. XM_008766702.2 (SEQ ID NO: 8).

The term “antibody” is used herein in its broadest sense and includes certain types of immunoglobulin molecules comprising one or more antigen-binding domains that specifically bind to an antigen or epitope. The term antibody as used herein refers to a molecule comprising at least complementarity-determining region (CDR) 1, CDR2, and CDR3 of a single domain antibody (sdAb), wherein the molecule is capable of binding to an antigen. The term antibody also refers to molecules comprising at least CDR1, CDR2, and CDR3 of a heavy chain and CDR1, CDR2, and CDR3 of a light chain, wherein the molecule is capable of binding to an antigen. The term antibody also includes fragments that are capable of binding an antigen, such as Fv, single-chain Fv (scFv), Fab, Fab′, and (Fab′)2. The term antibody also includes chimeric antibodies, humanized antibodies, and antibodies of various species such as mouse, human, cynomolgus monkey, llama, camel, etc. The term also includes multivalent antibodies such as bivalent or tetravalent antibodies. A multivalent antibody includes, e.g., a single polypeptide chain comprising multiple antigen binding (CDR-containing) domains, as well as two or more polypeptide chains, each containing one or more antigen binding domains, such two or more polypeptide chains being associated with one another, e.g., through a hinge region capable of forming disulfide bond(s) or any other covalent or noncovalent interaction.

The term “single domain antibody” or “sdAb” as used herein, refers to an antibody molecule or antigen binding fragment thereof comprising a single antigen binding domain sequence comprising a CDR1, CDR2, and CDR3, wherein the sdAb is capable of binding to antigen. Single domain antibodies may be derived from dromedary species, such as llama, camel, and alpaca, or from fish species. Alternatively, single domain antibodies may be obtained by laboratory techniques such as selection methods. In some embodiments, a sdAb may be humanized. In some embodiments, a sdAb may comprise part of a chimeric antibody or multivalent antibody.

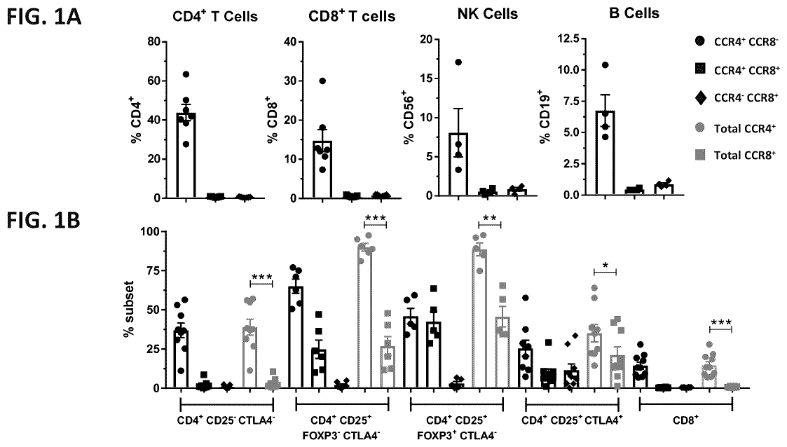

The term “heavy chain variable region” as used herein refers to a region comprising heavy chain CDR1, framework (FR) 2, CDR2, FR3, and CDR3. In some embodiments, a heavy chain variable region also comprises at least a portion of an FR1 and/or at least a portion of an FR4. In some embodiments, a heavy chain CDR1 corresponds to Kabat residues 26 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.); and FIG. 1. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See id.

The term “heavy chain constant region” as used herein refers to a region comprising at least three heavy chain constant domains, CH1, CH2, and CH3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an al constant region) and IgA2 (comprising an α2 constant region) antibodies; and IgM antibodies include, but are not limited to, IgM1 and IgM2.

The term “heavy chain” (abbreviated HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR)2, CDR2, FR3, and CDR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

The term “light chain constant region” as used herein refers to a region comprising a light chain constant domain, CL. Nonlimiting exemplary light chain constant regions include λ and κ.

The term “light chain” (abbreviate LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “isolated antibody”, as used herein, is intended to refer to an antibody that is substantially free of other antibodies having different antigenic specificities (e.g., an isolated antibody that specifically binds CCR8 is substantially free of antibodies that specifically bind antigens other than CCR8). An isolated antibody that specifically binds CCR8 may, however, have cross-reactivity to other antigens, such as CCR8 molecules from other species. Moreover, an isolated antibody may be substantially free of other cellular material and/or chemicals.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a sdAb, a Fab, an scFv, a (Fab′)2, etc. The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

A “CDR-grafted antibody” as used herein refers to a humanized antibody in which the complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The terms “multivalent” or “polyvalent” antibody, as used herein, refer interchangeably to antibodies comprising more than one antigen binding domain, such as two (“bivalent”) or four (“tetravalent”) antigen binding domains. In some embodiments, the two or more antigen binding domains may be identical in amino acid sequence. In other embodiments, the antigen binding domains may differ in amino acid sequence. In some embodiments, a multivalent antibody comprises two or more sdAb variable regions, while in some embodiments, a multivalent antibody comprises two or more sets of heavy and light chain variable regions.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

The terms “an anti-CCR8 antibody” and “an anti-C—C chemokine receptor 8 antibody”, used interchangeably herein, refer to an antibody that specifically binds to CCR8, e.g., human CCR8. An antibody “which binds” an antigen of interest, i.e., CCR8, is one capable of binding that antigen with sufficient affinity such that the antibody is useful in targeting a cell expressing the antigen. In a preferred embodiment, the antibody specifically binds to human CCR8 (hCCR8). Examples of anti-CCR8 antibodies are disclosed in the Examples, below. Unless otherwise indicated, the term “anti-CCR8 antibody” is meant to refer to an antibody which binds to wild type CCR8, a variant, or an isoform of CCR8.

In one embodiment, the phrase “specifically binds to hCCR8” or “specific binding to hCCR8”, as used herein, refers to the ability of an anti-CCR8 antibody to interact with CCR8 (human or cynomolgus monkey CCR8) with a dissociation constant (K_{D}) of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less. In another embodiment, the phrase “specifically binds to hCCR8” or “specific binding to hCCR8”, as used herein, refers to the ability of an anti-CCR8 antibody to interact with hCCR8 with a dissociation constant (K_{D}) of between about 1 pM (0.001 nM) to 2,000 nM, between about 500 pM (0.5 nM) to 1,000 nM, between about 500 pM (0.5 nM) to 500 nM, between about 1 nM) to 200 nM, between about 1 nM to 100 nM, between about 1 nM to 50 nM, between about 1 nM to 20 nM, or between about 1 nM to 5 nM. In one embodiment, K_{D} is determined by surface plasmon resonance or by any other method known in the art.

The term “antibody-dependent cell mediated cytotoxicity” or “ADCC” or “antibody-dependent cellular cytotoxicity” refers to a mechanism of cell-mediated immune defense through which Fc receptor-bearing effector cells can recognize and kill antibody-coated target cells expressing tumor- or pathogen-derived antigens on their surface. Specifically, recruitment of the effect cell to the target cell is mediated by the interaction between the Fc receptor expressed on the effector cell and the Fc region of an antibody bound with a cell surface antigen on the target cell, e.g., a tumor infiltrating Treg cell. Once the Fc receptor binds to the Fc region of the antibody, the effector cell releases cytotoxic factors that cause the death of the target cell. Non-limiting examples of effector cells include natural killer (NK) cells, macrophages, neutrophils and eosinophils.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

The term “epitope” refers to a region of an antigen that is bound by an antibody, or an antibody fragment. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, NJ). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₒₙ” or “kₐ”, as used herein, is intended to refer to the on rate constant for association of an antibody to the antigen to form the antibody/antigen complex.

The term “k_{off}” or “k_{d}”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction. K_{D} is calculated by kₐ/k_{d}. In one embodiment, the antibodies of the invention have a K_{D} of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about nM or less, or about 0.001 nM or less.

The term “labeled antibody” as used herein, refers to an antibody, or an antigen binding portion thereof, with a label incorporated that provides for the identification of the binding protein, e.g., an antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The term “antibody drug conjugate” refers to an ADC comprising an antibody, or antigen-binding portion thereof, that specifically binds to CCR8, whereby the antibody is conjugated to one or more chemical agent(s) or payloads. In one embodiment, the chemical agent is linked to the antibody via a linker.

As used herein, the term “effector cell” refers to a type of cells in the immune system that mediates an immune response against an antigen. Exemplary effector cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. In preferred embodiments, an effector cell is capable of inducing antibody-dependent cellular toxicity (ADCC), e.g., a natural killer cell or a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. In some embodiments, an effector cell can phagocytose a target antigen or a target cell. In other embodiments, an effector cell can lyse a target cell.

As used herein, the term “T cell” refers to a lymphocyte (e.g., white blood cell) that functions in cell-mediated immunity. In some embodiments, the presence of a T cell receptor (TCR) on the cell surface distinguishes T cells from other lymphocytes. As is known in the art, T cells typically do not present antigens, and rely on other lymphocytes (e.g., natural killer cells and B cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NK cells), gamma delta T cells, and Mucosal associated invariant T cells (MALT).

As used herein, the term “Treg cell” refers to Regulatory T cells (Treg), also sometimes referred to as Suppressor T cells. Treg cells maintain immunological tolerance. During an immune response, Tregs stop T cell-mediated immunity and suppress auto-reactive T cells that have escaped negative selection within the thymus. Treg cells have also been described as able to suppress other types of immune cells such as NK cells and B cells. There are two major classifications of Treg: natural Treg and peripheral Treg. Natural Treg cells are a class of thymically generated T-cells, while peripheral Treg develop in the periphery from naïve T cells in response to signals such as low doses of antigen, presence of certain microbes, lymphopenia or, in some cases, through activation by immature dendritic cells. In some cases, peripheral Treg are thought to be generated in response to inflammatory conditions, particularly those which may be due at least in part to the absence of natural Treg cells. Previous studies have shown that accumulation of Treg cells that have infiltrated into human tumors can block antitumor immunity, and thus enhance tumor progression. The presence of tumor infiltrating Tregs in the tumor microenvironment (TME) is also linked with unfavorable prognosis of cancer (Kim J H et al., Immune Netw. 2020 February; 20(1): e4.). Therefore, Tregs, particularly tumor infiltrating Tregs, are a key factor of hindrance in anti-tumor immunity in various types of cancer patients.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, myeloma and leukemia. In some embodiments, the cancer comprises a solid tumor cancer. In other embodiments, the cancer comprises a blood based cancer, e.g., leukemia, lymphoma or myeloma. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

In one embodiment, the antibodies of the invention are administered to a patient having a solid tumor, including an advanced solid tumor. In other embodiments, the antibodies of the invention are administered to a patient having a blood based cancer. In another embodiment, administration of the antibodies of the invention induce cell death of CCR8 expressing cells, e.g., tumor infiltrating Treg cells, and/or reduce or inhibit tumor growth or tumor volume. In some embodiments, the tumor growth or tumor volume is reduced by at least 10%, 15%, 20%, 25%, 30%, 35%, 40%, 45%, 50%, 55%, 60%, 65%, 70%, 75%, 80%, 85%, 90%, 95% or 100%. In some embodiments, administration of the antibodies of the invention results in complete regression of tumor growth.

The term “sample,” as used herein, refers to a composition that is obtained or derived from a subject that contains a cellular and/or other molecular entity that is to be characterized, quantitated, and/or identified, for example based on physical, biochemical, chemical and/or physiological characteristics. An exemplary sample is a tissue sample.

The term “tissue sample” refers to a collection of similar cells obtained from a tissue of a subject. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, synovial fluid, or interstitial fluid; cells from any time in gestation or development of the subject. The tissue sample may also be primary or cultured cells or cell lines. Optionally, the tissue sample is obtained from a disease tissue/organ, e.g. a tumor biopsy or synovial biopsy tissue sample. The tissue sample may contain compounds that are not naturally intermixed with the tissue in nature such as preservatives, anticoagulants, buffers, fixatives, nutrients, antibiotics, or the like. A “control sample” or “control tissue”, as used herein, refers to a sample, cell, or tissue obtained from a source known, or believed, not to be afflicted with the disease for which the subject is being treated.

For the purposes herein a “section” of a tissue sample means a part or piece of a tissue sample, such as a thin slice of tissue or cells cut from a solid tissue sample.

“Administering” refers to the physical introduction of a composition comprising a therapeutic agent to a subject, using any of the various methods and delivery systems known to those skilled in the art. Routes of administration for antibodies disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intratumoral, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include a topical, epidermal or mucosal route of administration, for example, orally, intranasally, vaginally, rectally, sublingually or topically. Administering can also be performed, for example, once, a plurality of times, and/or over one or more extended periods.

“Treatment,” as used herein, refers to therapeutic treatment, for example, wherein the object is to slow down (lessen) the targeted pathologic condition or disorder as well as, for example, wherein the object is to inhibit recurrence of the condition or disorder. In certain embodiments, the term “treatment” covers any administration or application of a therapeutic for disease in a patient, and includes inhibiting or slowing the disease or progression of the disease; partially or fully relieving the disease, for example, by causing regression, or restoring or repairing a lost, missing, or defective function; stimulating an inefficient process; or causing the disease plateau to have reduced severity. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down. In one embodiment, the symptoms of a disease or disorder, or pain and distress associated with an infection, are alleviated by at least 5%, at least 10%, at least 20%, at least 30%, at least 40%, at least 50%, at least 60%, at least 70%, at least 80%, at least 90%, or at least 95%.

Administration of a therapeutic agent “in combination with” one or more further therapeutic agents includes simultaneous (concurrent) and consecutive (sequential) administration in any order. For example, “concurrent” administration herein comprises administration of two or more agents on the same day, for example, during a single clinic, outpatient, or hospital visit. “Consecutive” or “sequential” administration herein means administration of two or more agents on different days.

The term “combination therapy”, as used herein, refers to the administration of two or more therapeutic substances, e.g., an anti-CCR8 antibody and an additional therapeutic agent. The additional therapeutic agent may be administered simultaneously (concomitant with), or consecutively (sequentially, e.g., prior to, or following the administration of the anti-CCR8 antibody). For example, “concurrent” administration herein comprises administration of two or more agents on the same day, for example, during a single clinic, outpatient, or hospital visit. “Consecutive” or “sequential” administration herein means administration of two or more agents on different days.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug. “Promoting cancer regression” means that administering an effective amount of the drug, alone or in combination with another anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction.

By way of example for the treatment of tumors, a therapeutically effective amount of an anti-cancer agent may inhibit cell growth, inhibit tumor growth, or reduce tumor size by at least about 5%, at least about 10%, by at least about 15%, at least about 20%, by at least about 25%, by at least about 30%, by at least about 40%, by at least about 50%, by at least about 60%, by at least about 70%, or by at least about 80%, by at least about 90%, by at least about 95%, or by at least about 100% relative to untreated subjects, relative to baseline, or, in certain embodiments, relative to patients treated with a standard-of-care therapy.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The term “increase” in the context, e.g., of a disease symptom, such as for example, tumor growth, refers to a statistically significant increase in such level. The increase can be, for example, at least 5%, 10%, 15%, 20%, 25%, 30%, 35%, 40%, 45%, 50%, 55%, 60%, 65%, 70%, 75%, 80%, 85%, 90%, or 95%, or above the level of detection for the detection method. The increase can also be, for example, about 1-10%, 10-20%, 1-30%, 20-50%, 30-60%, 40-70%, 50-80%, or 60-90% above the level of detection for the detection method. In certain embodiments, the increase is up to a level accepted as within the range of normal for an individual without such disorder which can also be referred to as a normalization of a level. In certain embodiments, the increase is the normalization of the level of a sign or symptom of a disease, an increase in the difference between the subject level of a sign of the disease and the normal level of the sign for the disease.

The term “decrease”, as used herein, in the context of a disease symptom refers to a statistically significant decrease in such level. The decrease can be, for example, at least 5%, 10%, 15%, 20%, 25%, 30%, 35%, 40%, 45%, 50%, 55%, 60%, 65%, 70%, 75%, 80%, 85%, 90%, or 95%, or below the level of detection for the detection method. The decrease can also be, for example, about 1-10%, 10-20%, 1-30%, 20-50%, 30-60%, 40-70%, 50-80%, or 60-90% below the level of detection for the detection method. In certain embodiments, the reduction is down to a level accepted as within the range of normal for an individual without such disorder which can also be referred to as a normalization of a level.

The term “control level” refers to an accepted or pre-determined level of a biological marker, e.g., the size of tumor obtained before administration of an antibody or an antigen-binding portion thereof. The level of a biological marker present in a subject or population of subjects having one or more particular characteristics, e.g., the presence or absence of a particular disease or condition.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

Various aspects of the invention are described in further detail in the following subsections.

### II. Anti-CCR8 Antibodies

The present invention is based, at least in part, on the development of engineered anti-CCR8 antibodies that have an enhanced ADCC activity. The inventors have successfully demonstrated in the working examples that CCR8 is a specific target for tumor infiltrating regulatory T cells (Treg), and treatment of the anti-CCR8 antibodies of the present invention can selectively deplete intratumoral or tumor infiltrating Treg cells, while have no effect on peripheral Treg cells. As a result, treatment with the anti-CCR8 antibodies of the present invention resulted in a selective depletion of tumor infiltrating Treg cells, and a significant reduction in tumor size and/or tumor growth in mouse tumor models. In addition, the present inventors have also demonstated that treatment with the anti-CCR8 antibodies promotes the development of an antigen-specific memory response.

Accordingly, the present invention provides anti-CCR8 antibodies, or antigen-binding fragments thereof. In one embodiment, the antibodies disclosed herein bind human CCR8. In another embodiment, the antibodies disclosed herein bind cynomolgus monkey CCR8. In another embodiment, the antibodies disclosed herein bind human CCR8 expressed on tumor infiltrating Treg cells and are capable of selectively depleting tumor infiltrating Treg cells, thereby preventing or reducing tumor growth.

The antibodies disclosed herein have characteristics including, but not limited to, binding to human and/or cynomolgus monkey CCR8 in vitro, inducing cytotoxicity in cells expressing CCR8, including, but not limited to, tumor infiltrating Treg cells, and decreasing or inhibiting cancer, tumor cellular proliferation or tumor growth, or tumor invasion and metastasis in vivo.

In some embodiments, the antibody or antigen-binding fragment thereof specifically binds to human CCR8 and/or Cynomolgus CCR8. In some embodiments, the antibody or antigen-binding fragment thereof has a dissociation constant (K_{d}) for human CCR8 less than 10 nM, and/or a dissociation constant (K_{d}) for Cynomolgus CCR8 less than 10 nM. In some embodiments, the antibody or antigen-binding fragment thereof does not bind to murine CCR8.

In some embodiments, the antibody or antigen-binding fragment thereof induces Fc receptor activation. In other embodiments, the antibody or antigen-binding fragment thereof induces Fc receptor activation with an EC50 less than 3 nM.

In some embodiments, the antibody or antigen-binding fragment thereof induces natural killer cell-mediated killing against cells expressing CCR8, e.g., tumor infiltrating cells. In some embodiments, the antibody or antigen-binding fragment thereof induces natural killer cell-mediated killing against cells expressing CCR8, e.g., tumor infiltrating cells, with an EC50 less than less than 1 nM,

In one embodiment, an anti-CCR8 antibody disclosed herein is capable of inducing cytotoxicity of a cell expressing CCR8, e.g., tumor infiltrating Treg cells. In one embodiment, an anti-CCR8 antibody disclosed herein is not being internalized into a cell expressing CCR8 or an effector cell. The anti-CCR8 antibodies disclosed herein are highly specific for intratumoral Treg cells, and have no effect on peripheral blood or spleenic Treg cells.

In some embodiments, an anti-CCR8 antibody, or fragment thereof, comprises any appropriate isotype, including, for example: IgG (e.g., IgG1, IgG2, IgG3, IgG4), IgM, IgA1, IgA2, IgD, or IgE. In some embodiments, an antibody, or fragment thereof, is an IgG isotype, e.g., IgG1.

The anti-CCR8 antibodies of the present invention have an enhanced antibody-dependent cell mediated cytotoxicity (ADCC) activity. Specifically, the anti-CCR8 antibodies have been engineered to possess an enhanced ADCC activity against cells expressing CCR8, e.g., tumor infiltrating Treg cells. Upon binding of the anti-CCR8 antibodies to the antigen expressed on the tumor infiltrating Treg cells, effector cells of the immune system are recruited to the tumor infiltrating Treg cells via the interaction between the Fc receptor expressed on the effector cells and the Fc region of the antibodies bound with CCR8. Once the Fc receptor binds to the Fc region of the antibody, the effector cells release cytotoxic factors that cause the death of the tumor infiltrating Treg cells, thereby specifically eliminating tumor infiltrating Treg cells. In some embodiments, the anti-CCR8 antibodies of the present invention suppress the intratumoral accumulation of Treg cells. In other embodiments, the anti-CCR8 antibodies of the present invention have an effect of removing or reducing tumor-infiltrating Treg cells, thereby treating cancer and/or reducing tumor growth.

In some embodiments, the anti-CCR8 antibodies have been engineered to improve Fc affinity for the activating Fc receptors on the effector cells. The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. The improvement of Fc affinity for FcγRs has led to increased ADCC activity, i.e., inducing cytotoxicity by releasing cytotoxic factors, such as granzymes and perforins, and depleting or removing target cells, e.g., CCR8 expressing cells, e.g., tumor infiltrating Treg cells.

Various approaches for Fc engineering are known in the art. For example, multiple mutations within the Fc domain have been identified that either directly or indirectly enhance binding of Fc receptors and through this significantly enhance cellular cytotoxicity or improve ADCC activity (Lazar, G. A., et al. (2006). PNAS 103, 4005-4010; Shields, R. L., et al. (2001). J. Biol. Chem. 276, 6591-6604; Stewart, R., et al. (2011). Protein Engineering, Design and Selection 24, 671-678; Richards, J. O., et al. (2008). Mol Cancer Ther 7, 2517-2527). In some embodiments, the Fc receptor is a FcγR, such as FcγRIIIa. Non-limiting examples of Fc domain mutations that enhance binding to Fc receptors include S239D/A330L/I332E (dubbed 3M), S298A/E333A/K334A (AAA), S239D/I332E, F243L, R292P, Y300L, V305I, P396L, M252Y, S254T, T256E, M428L, N434S, M252I, T256D, M428L And G236A (Saunders K O, Front Immunol. 2019; 10: 1296; Dall'Acqua et al 2006, J. Biol Chem Vol. 281(33) 23514-23524; Zalevsky et al 2010 Nature Biotech, Vol. 28(2) 157-159), the entire contents of each of the references are hereby incorporated by reference).

In some embodiments, the anti-CCR8 antibodies comprise one or more of the Fc domain mutations, as described herein. In some embodiments, the anti-CCR8 antibodies comprise one or more of the S239D/A330L/I332E mutations, e.g., the S239D/A330L/I332E mutations. In one embodiment, the antibody, or antigen binding fragment thereof, comprises the S239D mutation. In one embodiment, the antibody, or antigen binding fragment thereof, comprises the A330L mutation. In another embodiment, the antibody, or antigen binding fragment thereof, comprises the I332E mutation. In one embodiment, the antibody, or antigen binding fragment thereof, comprises the S239D/A330L mutations. In another embodiment, the antibody, or antigen binding fragment thereof, comprises the S239D/I332E mutations. In another embodiment, the antibody, or antigen binding fragment thereof, comprises the A330L/I332E mutations. In one embodiment, the antibody has each of the S239D/A330L/I332E mutations.

Another alternative approach to enhance antibody effector functions has focussed on glycosylation of the Fc domain. It is known that Fc receptors, e.g., FcγRs, interact with the carbohydrates on the CH2 domain and that the composition of these glycans has a substantial effect on effector function activity. Previously studies have shown that afucosylated (non-fucosylated) antibodies, which exhibit greatly enhanced ADCC activity through increased binding to FcγRIIIa (Jefferis, R. (2009). Methods Mol. Biol. 483, 223-238; Niwa, R., et al. (2004). Clin. Cancer Res. 10, 6248-6255; Okazaki, A., (2004). J. Mol. Biol. 336, 1239-1249; Ferrara, C., (2006). J. Biol. Chem. 281, 5032-5036; Yamane-Ohnuki, N., and Satoh, M. (2009). MAbs 1, 230-236).7-10).

In some embodiments, the anti-CCR8 antibodies have a mutation at the O- or N-linked glycosylation site. In another embodiment, the glycosylation of the anti-CCR8 antibody or antigen binding portion is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, a modified anti-CCR8 antibody can be made that has an altered type of glycosylation, such as a hypofucosylated antibody having reduced amounts of fucosyl residues or an antibody having increased bisecting GlcNAc structures. In some embodiments, the anti-CCR8 antibodies are not fucosylated. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety.

In addition to increasing affinity for receptors by introducing point mutations or modifying glycans, the Fc can be optimized by exchanging Fc domains across isotypes. Therefore, by creating a Fc region that can interact with multiple Fc receptors, one creates an antibody with expanded, novel abilities to engage effector cells. In some embodiments, the anti-CCR8 antibodies of the present invention comprise a chimeric heavy chain constant (CH) region, wherein the chimeric CH region comprises segments derived from the CH regions of more than one immunoglobulin isotype. For example, the antibodies of the invention may comprise a chimeric CH region comprising part or all of a CH2 domain derived from a human lgG1, human lgG2 or human lgG4 molecule, combined with part or all of a CH3 domain derived from a human IgG1, human lgG2 or human lgG4 molecule.

In some embodiments, exchanging between isotypes may increase the binding affinity between the Fc domain of the antibodies and the Fc receptor presented on the effector cells. In some embodiments, the anti-CCR8 antibody or antigen binding fragment thereof, comprises an IgG isotype, e.g., IgG1 or IgG2a. In some embodiments, the anti-CCR8 antibody or antigen binding fragment thereof, comprises an IgG1 isotype.

The antibody molecules, antigen-binding proteins, e.g., antigen-binding fragments of an antibody, may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

Exemplary Humanized Anti-CCR8 Antibodies

In some embodiments, the anti-CCR8 antibody or antigen-binding fragment thereof is a humanized antibody or antigen-binding fragment thereof. Humanized antibodies may be useful as therapeutic molecules because humanized antibodies may reduce or eliminate the human immune response to non-human antibodies (such as the human anti-mouse antibody (HAMA) response), which can result in an immune response to an antibody therapeutic, and decreased effectiveness of the therapeutic.

In some embodiments, anti-CCR8 antibodies, or antigen binding fragments thereof, of the present invention and the nucleic acid molecules of the present invention that encode the antibodies, or antigen binding fragments thereof, include the CDR amino acid sequences, the heavy chain (VH) and light chain (VL) variable region sequences, and the framework sequences shown in Tables 1-3.

TABLE 1
Heavy Chain and Light Chain CDR Sequences

| Antibody | VH CDR1 | VH CDR2 | VH CDR3 | VL CDR1 | VL CDR2 | VL CDR3 |
|---|---|---|---|---|---|---|
| I2676 | GFTFSSY | AVISYDG | ARVRDRA | TLRSGIN | YKSDSDK | WHSSAR |
| | AMH | SNKYYA | FDI | VGTYRIY | QQGS | NWV |
| | (SEQ ID | DSVKG | (SEQ ID | (SEQ ID | (SEQ ID | (SEQ ID |
| | NO: 9) | (SEQ ID | NO: 11) | NO: 12) | NO: 13) | NO: 14) |
| | | NO: 10) | | | | |
| I2677 | SYGMH | VISYDGS | DRRGGG | TLRSGIN | YKSDSDK | MIWHSS |
| | (SEQ ID | NKYYAD | YGDY | VGTYRIY | QQGS | ARNWV |
| | NO: 15) | SVKG | (SEQ ID | (SEQ ID | (SEQ ID | (SEQ ID |
| | | (SEQ ID | NO: 17) | NO: 12) | NO: 13) | NO: 20) |
| | | NO: 16) | | | | |
| I3144 | SYAMH | VISYDGS | VRDRAFD | TLRSGIN | YKSDSDK | MIWHSS |
| | (SEQ ID | NKYYAD | I | VGTYRIY | QQGS | ARNWV |
| | NO: 21) | SVKG | (SEQ ID | (SEQ ID | (SEQ ID | (SEQ ID |
| | | (SEQ ID | NO: 23) | NO: 12) | NO: 13) | NO: 20) |
| | | NO: 16) | | | | |
| I3145 | SNYMS | VIYSGGS | GLGSADY | RSSQSLL | KVSIRDS | MQSTQ |
| | (SEQ ID | TYYADS | (SEQ ID | HSNGNT | (SEQ ID | WPIT |
| | NO: 27) | VKG | NO: 29) | YLN | NO: 31) | (SEQ ID |
| | | (SEQ ID | | (SEQ ID | | NO: 32) |
| | | NO: 28) | | NO: 30) | | |
| I3210 | GFTFSSY | AVISYDG | ARVRDRA | TLRSGIN | IIKSGSSD | WHSSAR |
| | AMH | SNKYYA | FDI | VGTYRIY | KQQGS | NWV |
| | (SEQ ID | DSVKG | (SEQ ID | (SEQ ID | (SEQ ID | (SEQ ID |
| | NO: 9) | (SEQ ID | NO: 11) | NO: 12) | NO: 37) | NO: 14) |
| | | NO: 10) | | | | |
| I3213 | GFTFSSY | AVISYDG | ARVRDRA | TLRSGIN | YKSDSDK | WHSSAR |
| | AMH | SNKYYA | FDI | LGTYRIY | QQGS | NWV |
| | (SEQ ID | DSVKG | (SEQ ID | (SEQ ID | (SEQ ID | (SEQ ID |
| | NO: 9) | (SEQ ID | NO: 11) | NO: 42) | NO: 13) | NO: 14) |
| | | NO: 10) | | | | |

TABLE 2
VH and VL sequences

| | Amino Acid Sequence | Nucleic Acid Sequence |
|---|---|---|
| Description | (SEQ ID NO) | (SEQ ID NO) |
| I2676 | QVQLVESGGGVVQPGRSLR | CAAGTGCAGCTGGTCGAGAGCGGAGGAGGAGTGGT |
| Heavy chain | LSCAASGFTFSSYAMHWVR | GCAGCCCGGCAGATCTCTGAGACTGAGCTGTGCCGC |
| variable region | QAPGKGLEWVAVISYDGSN | CTCCGGCTTCACCTTCAGCAGCTACGCCATGCACTG |
| (VH) | KYYADSVKGRFTISRDNSKN | GGTGAGACAAGCCCCCGGCAAGGGACTGGAATGGG |
| | TLYLQMNSLRAEDTAVYYC | TGGCCGTCATCTCCTACGACGGCTCCAACAAGTACT |
| | ARVRDRAFDIWGQGTMVTV | ACGCCGACAGCGTGAAGGGAAGATTCACCATCTCT |
| | SS | AGAGACAACAGCAAGAACACACTGTATCTGCAGAT |
| | (SEQ ID NO: 45) | GAACTCTCTGAGAGCTGAGGACACAGCCGTGTACTA |
| | | TTGCGCTAGGGTGAGAGATAGAGCCTTCGACATCTG |
| | | GGGCCAAGGCACCATGGTGACCGTGAGCTCA |
| | | (SEQ ID NO: 57) |
| I2676 | QAVLTQPASLSASPGASASL | CAAGCCGTGCTGACACAACCCGCCAGCCTCAGCGCC |
| Light chain | TCTLRSGINVGTYRIYWYQQ | AGCCCCGGCGCTAGCGCTTCTCTGACATGCACACTG |
| variable region | KPGSPPQYLLRYKSDSDKQQ | AGGTCCGGCATCAACGTGGGCACCTATAGAATCTAC |
| (VL) | GSGVPSRFSGSKDASANAGI | TGGTACCAGCAGAAACCCGGCTCCCCTCCTCAGTAT |
| | LLISGLQSEDEADYYCMIWH | CTGCTGAGGTACAAGTCCGATAGCGACAAGCAGCA |
| | SSARNWVFGGGTKLTVL | AGGCTCCGGCGTGCCTTCTAGATTTAGCGGCAGCAA |
| | (SEQ ID NO: 51) | GGATGCCAGCGCCAATGCCGGCATTCTGCTGATCAG |
| | | CGGACTGCAGAGCGAGGATGAGGCCGACTACTACT |
| | | GCATGATCTGGCACTCCAGCGCCAGAAACTGGGTGT |
| | | TCGGCGGCGGAACCAAGCTGACCGTGCTA (SEQ ID |
| | | NO: 58) |
| I2677 | EVQLVESGGGVVQPGRSLRL | GAGGTGCAGCTGGTGGAAAGCGGAGGCGGAGTGGT |
| Heavy chain | SCAASGFTFSSYGMHWVRQ | GCAGCCCGGCAGATCTCTGAGGCTGAGCTGTGCCGC |
| variable region | APGKGLEWVAVISYDGSNK | TAGCGGCTTCACCTTCAGCAGCTACGGCATGCACTG |
| (VH) | YYADSVKGRFTISRDNSKNT | GGTGAGGCAAGCCCCCGGCAAGGGACTGGAGTGGG |
| | LYLQMNSLRAEDTAVYYCA | TCGCCGTGATCAGCTACGACGGCAGCAACAAGTACT |
| | KDRRGGGYGDYWGQGTLV | ACGCCGACAGCGTGAAGGGAAGATTCACCATCTCT |
| | TVSS | AGAGACAACAGCAAGAACACCCTCTACCTCCAGAT |
| | (SEQ ID NO: 46) | GAACTCTCTGAGGGCCGAGGATACCGCCGTGTACTA |
| | | CTGCGCCAAGGACAGAAGAGGCGGCGGATACGGCG |
| | | ATTACTGGGGCCAAGGCACACTGGTGACAGTGAGC |
| | | TCA |
| | | (SEQ ID NO: 59) |
| I2677 | QAVLTQPASLSASPGASASL | CAAGCCGTGCTGACCCAGCCCGCCTCTCTGAGCGCT |
| Light chain | TCTLRSGINVGTYRIYWYQQ | AGCCCCGGCGCCTCCGCTTCTCTGACATGCACACTG |
| variable region | KPGSPPQYLLRYKSDSDKQQ | AGGTCCGGAATCAACGTGGGCACCTATAGAATCTAC |
| (VL) | GSGVPSRFSGSKDASANAGI | TGGTACCAGCAGAAGCCCGGCAGCCCTCCTCAGTAT |
| | LLISGLQSEDEADYYCMIWH | CTGCTGAGATACAAGAGCGACAGCGATAAGCAGCA |
| | SSARNWVFGGGTQLTVL | AGGCTCCGGAGTGCCTAGCAGATTCAGCGGCAGCA |
| | (SEQ ID NO: 52) | AAGACGCCAGCGCCAATGCCGGAATTCTGCTGATCA |
| | | GCGGACTGCAGAGCGAGGACGAAGCCGACTACTAC |
| | | TGCATGATCTGGCACTCCAGCGCCAGAAACTGGGTG |
| | | TTTGGCGGCGGCACCCAGCTGACAGTGCTA |
| | | (SEQ ID NO: 60) |
| I3144 | QVQLVESGGGVVQPGRSLR | CAAGTGCAGCTGGTCGAGAGCGGAGGAGGAGTGGT |
| Heavy chain | LSCAASGFTFSSYAMHWVR | GCAGCCCGGCAGATCTCTGAGGCTGAGCTGCGCCGC |
| variable region | QAPGKGLEWVAVISYDGSN | CAGCGGATTCACCTTCAGCTCCTACGCCATGCACTG |
| (VH) | KYYADSVKGRFTISRDNSKN | GGTGAGACAAGCCCCCGGCAAGGGACTGGAGTGGG |
| | TLYLQMNSLRAEDTAVYYC | TGGCCGTGATTTCCTACGACGGCTCCAACAAGTACT |
| | ARVRDRAFDIWGQGTMVTV | ACGCCGACAGCGTGAAGGGAAGATTCACCATCTCT |
| | SS | AGAGACAACAGCAAGAACACACTGTATCTGCAGAT |
| | (SEQ ID NO: 45) | GAACTCTCTGAGAGCCGAGGACACCGCCGTGTACTA |
| | | CTGCGCCAGAGTGAGGGACAGAGCCTTCGACATTTG |
| | | GGGCCAAGGCACCATGGTGACAGTGAGCTCA |
| | | (SEQ ID NO: 61) |
| I3144 | QAVLTQPASLSASPGASASL | CAAGCCGTGCTGACCCAGCCCGCCTCTCTGAGCGCT |
| Light chain | TCTLRSGINVGTYRIYWYQQ | AGCCCCGGCGCTAGCGCTTCTCTGACATGCACACTG |
| variable region | KPGSPPQYLLRYKSDSDKQQ | AGGAGCGGCATCAACGTGGGCACCTATAGAATCTA |
| (VL) | GSGVPSRFSGSKDASANAGI | CTGGTACCAGCAGAAGCCCGGCAGCCCTCCTCAGTA |
| | LLISGLQSEDEADYYCMIWH | TCTGCTGAGATACAAGTCCGACAGCGACAAGCAGC |
| | SSARNWVFGGGTKLTVL | AAGGCAGCGGCGTGCCTTCTAGATTCAGCGGCAGC |
| | (SEQ ID NO: 51) | AAGGACGCCAGCGCTAATGCCGGCATTCTGCTGATC |
| | | AGCGGACTGCAGAGCGAGGATGAGGCCGACTACTA |
| | | CTGCATGATCTGGCACAGCAGCGCCAGAAACTGGG |
| | | TGTTCGGCGGCGGCACCAAGCTGACAGTGCTA |
| | | (SEQ ID NO: 62) |
| I3145 | EVQLVETGGGLIQPGGSLRL | GAGGTGCAGCTGGTGGAAACCGGCGGCGGACTGAT |
| Heavy chain | SCAASGFTVSSNYMSWVRQ | TCAGCCCGGAGGATCTCTGAGGCTGAGCTGTGCCGC |
| variable region | APGKGLEWVSVIYSGGSTYY | TAGCGGCTTCACCGTGAGCAGCAACTATATGAGCTG |
| (VH) | ADSVKGRFTISRDNSKNTLY | GGTGAGACAAGCCCCCGGCAAAGGACTGGAGTGGG |
| | LQMNSLRAEDTAVYYCARG | TGAGCGTGATCTACAGCGGCGGCAGCACATACTAC |
| | LGSADYWGQGTLVTVSS | GCCGACAGCGTGAAGGGAAGATTCACCATCTCTAG |
| | (SEQ ID NO: 48) | AGACAACAGCAAGAACACACTGTATCTGCAGATGA |
| | | ACTCTCTGAGGGCCGAGGACACCGCCGTGTACTACT |
| | | GCGCCAGAGGACTGGGCAGCGCTGATTACTGGGGC |
| | | CAAGGCACACTGGTGACAGTGTCCTCA |
| | | (SEQ ID NO: 63) |
| I3145 | DVVMTQSPLSLPVTLGQPAS | GACGTGGTGATGACCCAGAGCCCTCTGTCTCTGCCC |
| Light chain | ISCRSSQSLLHSNGNTYLNW | GTGACACTGGGACAGCCCGCCAGCATCAGCTGCAG |
| variable region | FQQRPGQSPRRLIYKVSIRDS | AAGCTCCCAGTCTCTGCTGCACAGCAATGGCAACAC |
| (VL) | GVPDRFSGSGSGTDFTLKISR | CTATCTGAACTGGTTCCAGCAAAGACCCGGCCAGTC |
| | VEAEDVGLYYCMQSTQWPI | CCCCAGAAGGCTGATCTACAAGGTGAGCATTAGAG |
| | TFGGGTKLEIK | ATAGCGGCGTGCCCGACAGATTTAGCGGCAGCGGA |
| | (SEQ ID NO: 54) | AGCGGCACAGACTTCACACTGAAGATCTCTAGAGTG |
| | | GAGGCTGAGGACGTGGGACTGTACTACTGCATGCA |
| | | GAGCACCCAGTGGCCCATCACCTTTGGCGGCGGCAC |
| | | CAAGCTGGAGATCAAA |
| | | (SEQ ID NO: 64) |
| I3210 | QVQLVESGGGVVQPGRSLR | CAAGTGCAGCTGGTCGAGAGCGGAGGAGGAGTGGT |
| Heavy chain | LSCAASGFTFSSYAMHWVR | GCAGCCCGGCAGATCTCTGAGGCTGAGCTGCGCCGC |
| variable region | QAPGKGLEWVAVISYDGSN | CAGCGGATTCACCTTCAGCTCCTACGCCATGCACTG |
| (VH) | KYYADSVKGRFTISRDNSKN | GGTGAGACAAGCCCCCGGCAAGGGACTGGAGTGGG |
| | TLYLQMNSLRAEDTAVYYC | TGGCCGTGATTTCCTACGACGGCTCCAACAAGTACT |
| | ARVRDRAFDIWGQGTMVTV | ACGCCGACAGCGTGAAGGGAAGATTCACCATCTCT |
| | SS | AGGGACAACAGCAAGAACACACTGTATCTGCAGAT |
| | (SEQ ID NO: 45) | GAACTCTCTGAGAGCCGAGGACACCGCCGTGTACTA |
| | | CTGCGCCAGAGTGAGGGACAGAGCCTTCGACATTTG |
| | | GGGCCAAGGCACCATGGTGACAGTGAGCTCA |
| | | (SEQ ID NO: 65) |
| I3210 | QAVLTQPASLSASPGASASL | CAAGCCGTGCTGACCCAGCCCGCCTCTCTGAGCGCT |
| Light chain | TCTLRSGINVGTYRIYWYQQ | AGCCCCGGCGCTAGCGCCTCTCTGACATGCACACTG |
| variable region | KPGSPPQYLLRIIKSGSSDKQ | AGAAGCGGCATCAACGTGGGCACCTATAGAATCTA |
| (VL) | QGSGVPSRFSGSKDASANAG | CTGGTACCAGCAGAAACCCGGCTCCCCC |
| | ILLISGLQSEDEADYYCMIW | CCTCAGTATCTGCTGAGAATCATCAAGAGCGGCAGC |
| | HSSARNWVFGGGTKLTVLG | AGCGACAAACAGCAAGGCAGCGGCGTGCCTAGCAG |
| | (SEQ ID NO: 55) | ATTCAGCGGCTCCAAGGATGCCAGCGCCAATGCCG |
| | | GCATTCTGCTGATCTCCGGACTGCAGAGCGAGGACG |
| | | AGGCCGACTACTACTGCATGATCTGGCACAGCTCCG |
| | | CCAGAAACTGGGTGTTCGGCGGCGGCACAAAGCTG |
| | | ACAGTGCTGGGC |
| | | (SEQ ID NO: 66) |
| I3213 | QVQLVESGGGVVQPGRSLR | CAAGTGCAGCTGGTCGAGAGCGGAGGAGGAGTGGT |
| Heavy chain | LSCAASGFTFSSYAMHWVR | GCAGCCCGGCAGATCTCTGAGGCTGAGCTGCGCCGC |
| variable region | QAPGKGLEWVAVISYDGSN | CAGCGGATTCACCTTCAGCTCCTACGCCATGCACTG |
| (VH) | KYYADSVKGRFTISRDNSKN | GGTGAGACAAGCCCCCGGCAAGGGACTGGAGTGGG |
| | TLYLQMNSLRAEDTAVYYC | TGGCCGTGATTTCCTACGACGGCTCCAACAAGTACT |
| | ARVRDRAFDIWGQGTMVTV | ACGCCGACAGCGTGAAGGGAAGATTCACCATCTCT |
| | SS | AGGGACAACAGCAAGAACACACTGTATCTGCAGAT |
| | (SEQ ID NO: 45) | GAACTCTCTGAGAGCCGAGGACACCGCCGTGTACTA |
| | | CTGCGCCAGAGTGAGGGACAGAGCCTTCGACATTTG |
| | | GGGCCAAGGCACCATGGTGACAGTGAGCTCA |
| | | (SEQ ID NO: 67) |
| I3213 | QAVLTQPASLSASPGASASL | CAAGCCGTGCTGACCCAGCCCGCCTCTCTGAGCGCT |
| Light chain | TCTLRSGINLGTYRIYWYQQ | AGCCCCGGCGCTTCCGCCTCTCTGACATGCACACTG |
| variable region | KPGSPPQYLLRYKSDSDKQQ | AGGTCCGGCATCAATCTGGGCACCTATAGAATCTAC |
| (VL) | GSGVPSRFSGSKDASANAGI | TGGTACCAGCAGAAGCCCGGCAGCCCTCCCCAGTAT |
| | LLISGLQSEDEADYYCMIWH | CTGCTGAGGTACAAGAGCGACAGCGATAAGCAGCA |
| | SSARNWVFGGGTKLTVLG | AGGCAGCGGCGTGCCTAGCAGATTTAGCGGAAGCA |
| | (SEQ ID NO: 56) | AGGACGCCTCCGCTAATGCCGGCATTCTGCTGATCA |
| | | GCGGACTGCAGAGCGAGGATGAGGCCGACTACTAC |
| | | TGCATGATCTGGCACTCCTCCGCCAGAAACTGGGTG |
| | | TTCGGCGGAGGCACCAAGCTGACAGTGCTGGGC |
| | | (SEQ ID NO: 68) |

TABLE 3
Framework Sequences

| | | | SEQ ID |
|---|---|---|---|
| Antibody | Region | Sequence | NO: |
| I2676 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |
| I2677 | VH FR1 | EVQLVESGGGVVQPGRSLRLSCAASGFTFS | 35 |
| | VH FR2 | WVRQAPGKGLEWVA | 36 |
| | VH FR3 | RFTISRDNSKNTLYLQMNSLRAEDTAVYYCAK | 38 |
| | VH FR4 | WGQGTLVTVSS | 39 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTQLTVL | 40 |
| I3144 | VH FR1 | QVQLVESGGGVVQPGRSLRLSCAASGFTFS | 41 |
| | VH FR2 | WVRQAPGKGLEWVA | 36 |
| | VH FR3 | RFTISRDNSKNTLYLQMNSLRAEDTAVYYCAR | 43 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVL | 44 |
| I3145 | VH FR1 | EVQLVETGGGLIQPGGSLRLSCAASGFTVS | 47 |
| | VH FR2 | WVRQAPGKGLEWVS | 53 |
| | VH FR3 | RFTISRDNSKNTLYLQMNSLRAEDTAVYYCAR | 43 |
| | VH FR4 | WGQGTLVTVSS | 39 |
| | VL FR1 | DVVMTQSPLSLPVTLGQPASISC | 49 |
| | VL FR2 | WFQQRPGQSPRRLIY | 50 |
| | VL FR3 | GVPDRFSGSGSGTDFTLKISRVEAEDVGLYYC | 69 |
| | VL FR4 | FGGGTKLEIK | 70 |
| I3210 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |
| I3213 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |

Nonlimiting exemplary humanized anti-CCR8 antibodies include I2676, I2677, I3144, I3145, I3210 and I3213, described herein. Nonlimiting exemplary humanized anti-CCR8 antibodies also include antibodies comprising a heavy chain variable region of an antibody selected from I2676, I2677, I3144, I3145, I3210 and I3213 and/or a light chain variable region of an antibody selected from I2676, I2677, I3144, I3145, I3210 and I3213. Nonlimiting exemplary humanized antibodies include antibodies comprising a heavy chain variable region selected from SEQ ID NOs: 45, 46 and 48 and/or a light chain variable region selected from SEQ ID NOs: 51, 52 and 54-56.

In some embodiments, a humanized anti-CCR8 antibody comprises heavy chain CDR1, CDR2, and CDR3 and/or a light chain CDR1, CDR2, and CDR3 of an antibody selected from I2676, I2677, I3144, I3145, I3210 and I3213. Nonlimiting exemplary humanized anti-CCR8 antibodies include antibodies comprising sets of heavy chain CDR1, CDR2, and CDR3 selected from: SEQ ID NOs: 9-11; SEQ ID NOs: 15-17; SEQ ID Nos: 21, 16 and 23, and SEQ ID Nos: 27-29. Nonlimiting exemplary humanized anti-CCR8 antibodies also include antibodies comprising sets of light chain CDR1, CDR2, and CDR3 selected from: SEQ ID NOs: 12-14; SEQ ID NOs: 12, 13 and 20; SEQ ID Nos: 30-32; SEQ ID Nos: 12, 37 and 14; and SEQ ID Nos: 42, 13 and 14.

In some embodiments, a humanized anti-CCR8 antibody, or antigen-binding fragment thereof, comprises a heavy chain comprising the amino acid sequence selected from SEQ ID NOs:71, 72, 74 and 75, and a light chain comprising the amino acid sequence selected from SEQ ID NO:73 and 76.

Further Exemplary Humanized Anti-CCR8 Antibodies

In some embodiments, a humanized anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48, and wherein the antibody binds CCR8. In some embodiments, a humanized anti-CCR8 antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56, wherein the antibody binds CCR8. In some embodiments, a humanized anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56; wherein the antibody binds CCR8.

In some embodiments, a humanized anti-CCR8 antibody comprises at least one of the CDRs discussed herein. That is, in some embodiments, an anti-CCR8 antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein.

Further, in some embodiments, a humanized anti-CCR8 antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary humanized anti-CCR8 antibodies also include humanized antibodies that compete for binding to CCR8 with an antibody described herein. Thus, in some embodiments, a humanized anti-CCR8 antibody is provided that competes for binding to CCR8 with an antibody selected from I2676, I2677, I3144, I3145, I3210 and I3213.

Exemplary Humanized Antibody Constant Regions

In some embodiments, a humanized antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a humanized antibody described herein comprises a human IgG constant region. In some embodiments, a humanized antibody described herein comprises a human IgG4 heavy chain constant region. In some such embodiments, a humanized antibody described herein comprises an S241P mutation (Kabat numbering) in the human IgG4 constant region. In some embodiments, a humanized antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, a humanized anti-CCR8 antibody comprising a human IgG1 heavy chain or a human IgG3 heavy chain is selected.

An anti-CCR8 antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

As noted above, a humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an anti-CCR8 antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized anti-CCR8 antibody also comprises a human heavy chain constant region and/or a human light chain constant region.

Exemplary Chimeric Anti-CCR8 Antibodies

In some embodiments, an anti-CCR8 antibody is a chimeric antibody. In some embodiments, an anti-CCR8 antibody comprises at least one non-human variable region and at least one human constant region. In some such embodiments, all of the variable regions of an anti-CCR8 antibody are non-human variable regions, and all of the constant regions of an anti-CCR8 antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

Nonlimiting exemplary chimeric antibodies include chimeric antibodies comprising the heavy and/or light chain variable regions of an antibody selected from 12676, 12677, 13144, 13145, 13210 and 13213. Additional nonlimiting exemplary chimeric antibodies include chimeric antibodies comprising heavy chain CDR1, CDR2, and CDR3, and/or light chain CDR1, CDR2, and CDR3 of an antibody selected from I2676, I2677, I3144, I3145, I3210 and I3213.

Nonlimiting exemplary chimeric anti-CCR8 antibodies include antibodies comprising the following pairs of heavy and light chain variable regions: SEQ ID NOs: 45 and 51; SEQ ID NOs: 46 and 52; SEQ ID NOs: 48 and 54; SEQ ID NOs: 45 and 55; and SEQ ID NOs: 45 and 56. Nonlimiting exemplary anti-CCR8 antibodies include antibodies comprising a set of heavy chain CDR1, CDR2, and CDR3, and light chain CDR1, CDR2, and CDR3 shown above in Table 1.

In some embodiments, a chimeric anti-CCR8 antibody comprises at least one of the CDRs discussed herein. That is, in some embodiments, a chimeric anti-CCR8 antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein.

In some embodiments, a chimeric anti-CCR8 antibody, or antigen-binding fragment thereof, comprises a heavy chain comprising the amino acid sequence selected from SEQ ID NOs:71, 72, 74 and 75, and a light chain comprising the amino acid sequence selected from SEQ ID NO:73 and 76.

Further Exemplary Chimeric Anti-CCR8 Antibodies

In some embodiments, a chimeric anti-CCR8 antibody comprises heavy chain CDR1, CDR2, and CDR3 and/or a light chain CDR1, CDR2, and CDR3 of an antibody selected from I2676, I2677, I3144, I3145, I3210 and I3213. Nonlimiting exemplary chimeric anti-CCR8 antibodies include antibodies comprising sets of heavy chain CDR1, CDR2, and CDR3 selected from: SEQ ID NOs: 9-11; SEQ ID NOs: 15-17; SEQ ID Nos: 21, 16 and 23, and SEQ ID Nos: 27-29. Nonlimiting exemplary chimeric anti-CCR8 antibodies also include antibodies comprising sets of light chain CDR1, CDR2, and CDR3 selected from: SEQ ID NOs: 12-14; SEQ ID NOs: 12, 13 and 20; SEQ ID Nos: 30-32; SEQ ID Nos: 12, 37 and 14; and SEQ ID Nos: 42, 13 and 14.

In some embodiments, a chimeric anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48, and wherein the antibody binds CCR8. In some embodiments, a chimeric anti-CCR8 antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56, wherein the antibody binds CCR8. In some embodiments, a chimeric anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56; wherein the antibody binds CCR8.

Further, in some embodiments, a chimeric anti-CCR8 antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary chimeric anti-CCR8 antibodies also include chimeric antibodies that compete for binding to CCR8 with an antibody described herein. Thus, in some embodiments, a chimeric anti-CCR8 antibody is provided that competes for binding to CCR8 with an antibody selected from I2676, I2677, I3144, I3145, I3210 and I3213.

Exemplary Anti-CCR8 Chimeric Antibody Constant Regions

In some embodiments, a chimeric antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a chimeric antibody described herein comprises a human IgG constant region, such as an IgG1, IgG2, IgG3, or IgG4 constant region. In some embodiments, a chimeric antibody described herein comprises a human IgG4 heavy chain constant region. In some such embodiments, a chimeric antibody described herein comprises a human IgG4 constant region with an S241P mutation. In some embodiments, a chimeric antibody described herein comprises a human IgG4 constant region and a human κ light chain.

As noted above, whether or not effector function is desirable may depend on the particular method of treatment intended for an antibody. Thus, in some embodiments, when effector function is desirable, a chimeric anti-CCR8 antibody comprising a human IgG1 heavy chain constant region or a human IgG3 heavy chain constant region is selected. In some embodiments, when effector function is not desirable, a chimeric anti-CCR8 antibody comprising a human IgG4 or IgG2 heavy chain constant region is selected.

Exemplary Anti-CCR8 Human Antibodies

Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

In some embodiments, a human anti-CCR8 antibody binds to CCR8. Exemplary human anti-CCR8 antibodies also include antibodies that compete for binding to CCR8 with an antibody described herein. Thus, in some embodiments, a human anti-CCR8 antibody is provided that competes for binding to CCR8 with an antibody selected from I2676, I2677, I3144, I3145, I3210 and I3213.

In some embodiments, a human anti-CCR8 antibody comprises one or more human constant regions. In some embodiments, the human heavy chain constant region is of an isotype selected from IgA, IgG, and IgD. In some embodiments, the human light chain constant region is of an isotype selected from κ and λ. In some embodiments, a human antibody described herein comprises a human IgG constant region, such as an IgG1, IgG2, IgG3, or IgG4 constant region. In some embodiments, a human antibody described herein comprises a human IgG4 heavy chain constant region. In some such embodiments, a human antibody described herein comprises a human IgG4 heavy chain constant region with an S241P mutation. In some embodiments, a human antibody described herein comprises a human IgG4 constant region and a human κ light chain.

In some embodiments, when effector function is desirable, a human anti-CCR8 antibody comprising a human IgG1 heavy chain constant region or a human IgG3 heavy chain constant region is selected. In some embodiments, when effector function is not desirable, a human anti-CCR8 antibody comprising a human IgG4 or IgG2 heavy chain constant region is selected.

Additional Exemplary Anti-CCR8 Antibodies

Exemplary anti-CCR8 antibodies also include, but are not limited to, mouse, humanized, human, chimeric, and engineered antibodies that comprise, for example, one or more of the CDR sequences described herein. In some embodiments, an anti-CCR8 antibody comprises a heavy chain variable region described herein. In some embodiments, an anti-CCR8 antibody comprises a light chain variable region described herein. In some embodiments, an anti-CCR8 antibody comprises a heavy chain variable region described herein and a light chain variable region described herein. In some embodiments, an anti-CCR8 antibody comprises heavy chain CDR1, CDR2, and CDR3 described herein. In some embodiments, an anti-CCR8 antibody comprises light chain CDR1, CDR2, and CDR3 described herein. In some embodiments, an anti-CCR8 antibody comprises heavy chain CDR1, CDR2, and CDR3 described herein and light chain CDR1, CDR2, and CDR3 described herein.

In some embodiments, an anti-CCR8 antibody comprises a heavy chain variable region of an antibody selected from I2676, I2677, I3144, I3145, I3210 and I3213. Nonlimiting exemplary anti-CCR8 antibodies include antibodies comprising a heavy chain variable region comprising a sequence selected from SEQ ID NOs: 45, 46 and 48.

In some embodiments, an anti-CCR8 antibody comprises a light chain variable region of an antibody selected from I2676, I2677, I3144, I3145, I3210 and I3213. Nonlimiting exemplary anti-CCR8 antibodies include antibodies comprising a light chain variable region comprising a sequence selected from SEQ ID NOs: 51, 52 and 54-56.

In some embodiments, an anti-CCR8 antibody comprises a heavy chain variable region and a light chain variable region of an antibody selected from I2676, I2677, I3144, I3145, I3210 and I3213. Nonlimiting exemplary anti-CCR8 antibodies include antibodies comprising the following pairs of heavy and light chain variable regions: SEQ ID NOs: 45 and 51; SEQ ID NOs: 46 and 52; SEQ ID NOs: 48 and 54; SEQ ID NOs: 45 and 55; and SEQ ID NOs: 45 and 56.

In some embodiments, an anti-CCR8 antibody comprises heavy chain CDR1, CDR2, and CDR3 of an antibody selected from I2676, I2677, I3144, I3145, I3210 and I3213. Nonlimiting exemplary anti-CCR8 antibodies include antibodies comprising sets of heavy chain CDR1, CDR2, and CDR3 selected from: SEQ ID NOs: 9-11; SEQ ID NOs: 15-17; SEQ ID Nos: 21, 16 and 23, and SEQ ID Nos: 27-29. Nonlimiting exemplary anti-CCR8 antibodies also include antibodies comprising sets of light chain CDR1, CDR2, and CDR3 selected from: SEQ ID NOs: 12-14; SEQ ID NOs: 12, 13 and 20; SEQ ID Nos: 30-32; SEQ ID Nos: 12, 37 and 14; and SEQ ID Nos: 42, 13 and 14.

Nonlimiting exemplary anti-CCR8 antibodies include antibodies comprising a set of heavy chain CDR1, CDR2, and CDR3, and light chain CDR1, CDR2, and CDR3 shown above in Table 1.

In some embodiments, a human anti-CCR8 antibody, or antigen-binding fragment thereof, comprises a heavy chain comprising the amino acid sequence selected from SEQ ID NOs:71, 72, 74 and 75, and a light chain comprising the amino acid sequence selected from SEQ ID NOs:73 and 76.

Further Exemplary Anti-CCR8 Antibodies

In some embodiments, an anti-CCR8 antibody comprises heavy chain CDR1, CDR2, and CDR3 and/or a light chain CDR1, CDR2, and CDR3 of an antibody selected from I2676, I2677, I3144, I3145, I3210 and I3213. Nonlimiting exemplary anti-CCR8 antibodies include antibodies comprising sets of heavy chain CDR1, CDR2, and CDR3 selected from: SEQ ID NOs: 9-11; SEQ ID NOs: 15-17; SEQ ID Nos: 21, 16 and 23, and SEQ ID Nos: 27-29. Nonlimiting exemplary anti-CCR8 antibodies also include antibodies comprising sets of light chain CDR1, CDR2, and CDR3 selected from: SEQ ID NOs: 12-14; SEQ ID NOs: 12, 13 and 20; SEQ ID Nos: 30-32; SEQ ID Nos: 12, 37 and 14; and SEQ ID Nos: 42, 13 and 14.

In some embodiments, an anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48, and wherein the antibody binds CCR8. In some embodiments, an anti-CCR8 antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56, wherein the antibody binds CCR8. In some embodiments, an anti-CCR8 antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56; wherein the antibody binds CCR8.

Further, in some embodiments, an anti-CCR8 antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary anti-CCR8 antibodies also include chimeric antibodies that compete for binding to CCR8 with an antibody described herein. Thus, in some embodiments, a chimeric anti-CCR8 antibody is provided that competes for binding to CCR8 with an antibody selected from I2676, I2677, I3144, I3145, I3210 and I3213.

Exemplary Anti-CCR8 Heavy Chain Variable Regions

In some embodiments, anti-CCR8 antibody heavy chain variable regions are provided. In some embodiments, an anti-CCR8 antibody heavy chain variable region is a mouse variable region, a human variable region, or a humanized variable region.

An anti-CCR8 antibody comprises a heavy chain variable region comprising a heavy chain CDR1, FR2, CDR2, FR3, and/or CDR3. In some embodiments, an anti-CCR8 antibody heavy chain variable region further comprises a heavy chain FR1 and/or FR4. Nonlimiting exemplary heavy chain variable regions include, but are not limited to, heavy chain variable regions having an amino acid sequence selected from SEQ ID NOs: 45, 46 and 48.

In some embodiments, an anti-CCR8 antibody comprises a heavy chain variable region comprising a CDR1 domain comprising an amino acid sequence selected from a group consisting of SEQ ID NOs: 11, 17, 23, and 29, or a substantially similar sequence thereof having at least 90%, at least 95%, at least 98% or at least 99% sequence identity.

In some embodiments, an anti-CCR8 antibody comprises a heavy chain variable region comprising a CDR2 domain comprising an amino acid sequence selected from a group consisting of SEQ ID NOs: 10, 16, and 28, or a substantially similar sequence thereof having at least 90%, at least 95%, at least 98% or at least 99% sequence identity.

In some embodiments, an anti-CCR8 antibody comprises a heavy chain variable region comprising a CDR3 domain comprising an amino acid sequence selected from a group consisting of SEQ ID NOs: 11, 17, 23, and 29, or a substantially similar sequence thereof having at least 90%, at least 95%, at least 98% or at least 99% sequence identity.

Nonlimiting exemplary heavy chain variable regions include, but are not limited to, heavy chain variable regions comprising sets of CDR1, CDR2, and CDR3 selected from: SEQ ID NOs: 9-11; SEQ ID NOs: 15-17; SEQ ID Nos: 21, 16 and 23, and SEQ ID Nos: 27-29.

In some embodiments, an anti-CCR8 antibody heavy chain comprises a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 45, 46 and 48, wherein the heavy chain, together with a light chain, is capable of forming an antibody that binds CCR8.

In some embodiments, an anti-CCR8 antibody comprises a heavy chain comprising at least one of the CDRs discussed herein. That is, in some embodiments, an anti-CCR8 antibody heavy chain comprises at least one CDR selected from a heavy chain CDR1 discussed herein, a heavy chain CDR2 discussed herein, and a heavy chain CDR3 discussed herein. Further, in some embodiments, an anti-CCR8 antibody heavy chain comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the heavy chain comprising the mutated CDR.

In some embodiments, a heavy chain comprises a heavy chain constant region. In some embodiments, a heavy chain comprises a human heavy chain constant region. In some embodiments, the human heavy chain constant region is of an isotype selected from IgA, IgG, and IgD. In some embodiments, the human heavy chain constant region is an IgG constant region. In some embodiments, a heavy chain comprises a human igG4 heavy chain constant region. In some such embodiments, the human IgG4 heavy chain constant region comprises an S241P mutation.

In some embodiments, when effector function is desirable, a heavy chain comprises a human IgG1 or IgG3 heavy chain constant region. In some embodiments, when effector function is less desirable, a heavy chain comprises a human IgG4 or IgG2 heavy chain constant region.

Exemplary Anti-CCR8 Light Chain Variable Regions

In some embodiments, anti-CCRI antibody light chain variable regions are provided. In some embodiments, an anti-CCR8 antibody light chain variable region is a mouse variable region, a human variable region, or a humanized variable region.

An anti-CCR8 antibody comprises a light chain variable region comprising a light chain CDR1, FR2, CDR2, FR3, and/or CDR3. In some embodiments, an anti-CCR8 antibody light chain variable region further comprises a light chain FR1 and/or FR4. Nonlimiting exemplary light chain variable regions include light chain variable regions having an amino acid sequence selected from SEQ ID NOs: 51, 52 and 54-56.

In some embodiments, an anti-CCR8 antibody comprises a light chain variable region comprising a CDR1 domain comprising an amino acid sequence selected from a group consisting of SEQ ID NOs: 12, 30, and 42, or a substantially similar sequence thereof having at least 90%, at least 95%, at least 98% or at least 99% sequence identity.

In some embodiments, an anti-CCR8 antibody comprises a light chain variable region comprising a CDR2 domain comprising an amino acid sequence selected from a group consisting of SEQ ID NOs: 13, 31, and 37, or a substantially similar sequence thereof having at least 90%, at least 95%, at least 98% or at least 99% sequence identity.

In some embodiments, an anti-CCR8 antibody comprises a light chain variable region comprising a CDR3 domain comprising an amino acid sequence selected from a group consisting of SEQ ID NOs: 14, 20, and 32, or a substantially similar sequence thereof having at least 90%, at least 95%, at least 98% or at least 99% sequence identity.

Nonlimiting exemplary light chain variable regions include, but are not limited to, light chain variable regions comprising sets of CDR1, CDR2, and CDR3 selected from: SEQ ID NOs: 12-14; SEQ ID NOs: 12, 13 and 20; SEQ ID Nos: 30-32; SEQ ID Nos: 12, 37 and 14; and SEQ ID Nos: 42, 13 and 14.

In some embodiments, an anti-CCR8 antibody light chain comprises a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 51, 52 and 54-56, wherein the light chain, together with a heavy chain, is capable of forming an antibody that binds CCR8.

In some embodiments, an anti-CCR8 antibody comprises a light chain comprising at least one of the CDRs discussed herein. That is, in some embodiments, an anti-CCR8 antibody light chain comprises at least one CDR selected from a light chain CDR1 discussed herein, a light chain CDR2 discussed herein, and a light chain CDR3 discussed herein. Further, in some embodiments, an anti-CCR8 antibody light chain comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the light chain comprising the mutated CDR.

In some embodiments, a light chain comprises a human light chain constant region. In some embodiments, a human light chain constant region is selected from a human κ and a human λ light chain constant region.

Exemplary Properties of Anti-CCR8 Antibodies

In some embodiments, an antibody having a structure described herein binds to the CCR8 with a binding affinity (KD) of less than 10 nM, induces Fc receptor activation, and/or induces natural killer (NK) cell-mediated killing against cells expressing CCR8, e.g., tumor infiltrating Treg cells.

In some embodiments, an anti-CCR8 antibody binds to CCR8 with a binding affinity (KD) less than 10 nM, less than 5 nM, less than 1 nM, less than 0.5 nM, less than 0.1 nM, or less than 0.05 nM. In some embodiments, an anti-CCR8 antibody has a KD of between 0.01 and 1 nM, between 0.01 and 0.5 nM, between 0.01 and 0.1 nM, between 0.01 and 0.05 nM, or between 0.02 and 0.05 nM.

In some embodiments, an anti-CCR8 antibody induces Fc receptor activation with an EC50 less than 3 nM, less than 2 nM, less than 1 nM, less than 0.5 nM, less than 0.1 nM, less than 0.05 nM or less than 0.01 nM. In some embodiments, the antibody or antigen-binding fragment thereof has an EC50 less than 100 pM. In other embodiments, the antibody or antigen-binding fragment thereof has an EC50 less than 10 pM. In some embodiments, an anti-CCR8 antibody has an EC50 of between 0.001 and 0.01 nM, between 0.01 and 1 nM, between 0.01 and 0.5 nM, between 0.01 and 0.1 nM, between 0.01 and 0.05 nM, or between and 0.05 nM. In some embodiments, the antibody or antigen-binding fragment thereof has an EC50 less than 3 nM in an in vitro Fc receptor activation assay. In one embodiment, Fc receptor activation is measured by a luciferase reporter assay, e.g., by incubating CCR8-expressing cells and Jurkat cells expressing FcγRIIIa and a luciferase gene under the control of the NFAT promoter (see, e.g., Example 10), or by any other method known in the art.

In some embodiments, an anti-CCR8 antibody induces natural killer cell-mediated killing against cells expressing CCR8, e.g., tumor infiltrating cells. In some embodiments, an anti-CCR8 antibody induces natural killer cell-mediated killing against cells expressing CCR8, e.g., tumor infiltrating cells, with an EC50 less than 1 nM, less than 0.1 nM, less than pM, less than 80 pM, less than 70 pM, less than 60 pM, less than 50 pM, less than 40 pM, less than 30 pM, less than 20 pM, less than 10 pM, less than 5 pM, less than 1 pM. In some embodiments, the antibody or antigen-binding fragment thereof induces natural killer cell-mediated killing against cells expressing CCR8 with an EC50 less than 100 pM. In other embodiments, the antibody or antigen-binding fragment thereof induces natural killer cell-mediated killing against cells expressing CCR8 with an EC50 less than 10 pM. In some embodiments, an anti-CCR8 antibody has an EC50 of between 1 pM and 10 pM, between 10 pM and 50 pM, between 10 pM and 100 pM, between 20 pM and 50 pM, or between 20 pM and 100 pM. In other embodiments, the antibody or antigen-binding fragment thereof has an EC50 less than 1 nM in an in vitro natural killer cell-mediated killing assay. In one embodiment, the antibody induced natural killer cell-mediated killing activity is measured by an in vitro assay, as described in e.g., Example 11 of the application, or by any other method known in the art.

In one embodiment, an anti-CCR8 antibody disclosed herein is not internalized into a cell expressing CCR8 or an effector cell. The anti-CCR8 antibodies disclosed herein are highly specific for intratumoral Treg cells, and have no effect on peripheral blood or spleenic Treg cells.

### Conjugates Containing Anti-CCR8 Antibodies of the Invention

In some embodiments, the anti-CCR8 antibody, or antibody portion thereof, of the present invention is derivatized or linked to one or more functional molecule(s) (e.g., another peptide or protein). For example, an antibody can be derived by functionally linking an antibody or antibody portion (by chemical coupling, genetic fusion, noncovalent association or otherwise) to one or more other molecular entities, such as another antibody (e.g., a bispecific antibody or a diabody; a trispecific antibody or a tribody, a tetraspecific antibody or a tetrabody), a detectable agent, a pharmaceutical agent, a protein or peptide that can mediate the association of the antibody or antibody portion with another molecule (such as a streptavidin core region or a polyhistidine tag). The antigen recognition domain, e.g., the single-chain variable fragment (scFv) of an antibody, may be linked to the scFv of another antibody to create a tandem scFV. Alternatively, the scFv of an antibody may be linked via CH3 domains to the scFv of another antibody, paired through heterodimerization, to create a minibody. In some embodiments, the anti-CCR8 antibody or antibody portion thereof is capable of recruiting and activating T cells, resulting in T cell mediated cytotoxicity. In other embodiments, the anti-CCR8 antibody or antibody portion thereof is capable of recruiting and activating natural killer (NK) cells, resulting in NK cell mediated cytotoxicity. For example, the anti-CCR8 antibody or antibody portion thereof may be linked to another antibody, e.g., an antibody targeting NKp30 receptor, which may result in recruitment and activation of natural killer cells.

In further embodiments, the anti-CCR8 antibodies described herein may be conjugated to a drug moiety, e.g., a a cytotoxic or therapeutic agent, to form an anti-CCR8 Antibody Drug Conjugate (ADC). Antibody-drug conjugates (ADCs) may increase the therapeutic efficacy of antibodies in treating disease, e.g., cancer, due to the ability of the ADC to selectively deliver one or more drug moiety(s) to target cells, e.g., CCR8 expressing cells, e.g., tumor infiltrating Treg cells. Thus, in certain embodiments, the present invention provides anti-CCR8 ADCs for therapeutic use, e.g., treatment of cancer.

Non-limiting examples of drugs that may be used in ADCs, i.e., drugs that may be conjugated to the anti-CCR8 antibodies, include mitotic inhibitors, antitumor antibiotics, immunomodulating agents, gene therapy vectors, alkylating agents, antiangiogenic agents, antimetabolites, boron-containing agents, chemoprotective agents, hormone agents, glucocorticoids, photoactive therapeutic agents, oligonucleotides, radioactive isotopes, radiosensitizers, topoisomerase inhibitors, tyrosine kinase inhibitors, and combinations thereof.

Useful detectable agents with which an antibody or antibody portion thereof, may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present the addition of hydrogen peroxide and diaminobenzidine leads to a colored reaction product, which is detectable. An antibody may also be derivatized with biotin, and detected through indirect measurement of avidin or streptavidin binding.

In one embodiment, the antibody is conjugated to an imaging agent. Examples of imaging agents that may be used in the compositions and methods described herein include, but are not limited to, a radiolabel (e.g., indium), an enzyme, a fluorescent label, a luminescent label, a bioluminescent label, a magnetic label, and biotin.

In some embodiments, the anti-CCR8 antibody, or antibody portion thereof, of the present invention is derivatized or linked to one or more functional molecule(s) for use in generating a Chimeric Antigen Receptor (CAR). As used herein, the term “chimeric antigen receptors (CARs, also known as chimeric immunoreceptors, chimeric T cell receptors or artificial T cell receptors),” refers to receptor proteins that have been engineered to give T cells the new ability to target a specific protein. These receptors are chimeric because they combine both antigen-binding and T-cell activating functions into a single receptor. CARs are generally composed of four regions: an antigen recognition domain, e.g., the single-chain variable fragment (scFv) of an antibody; an extracellular hinge region, a transmembrane domain, and an intracellular T-cell signaling domain. CARs link an extracellular antigen recognition domain to an intracellular signalling domain, which activates the T cell when an antigen is bound. In some embodiments, the scFv of an anti-CCR8 antibody of the present invention is linked to an extracellular hinge region, a transmembrane domain and an intracellular T-cell signaling domain in order to generate a chimeric antigen receptor. Alternatively, the CAR technology may be applied to other immune cells such as natural killer (NK) cells. For example, the NK cells may be engineered to express CARs comprising the scFv of the anti-CCR8 antibody.

In other embodiments, the anti-CCR8 antibody, or antibody portion thereof, of the present invention is derivatized or linked to one or more functional molecule(s) for use in generating a Bi-specific T-cell engager (BiTE). As used herein, the term “Bi-specific T-cell engager (BiTE)” refers to a class of artificial bispecific monoclonal antibodies that are investigated for use as anti-cancer drugs. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. In some embodiments, the scFv of an anti-CCR8 antibody of the present invention is linked to another scFv which binds to CD3.

Nucleic Acids Encoding anti-CCR8 Antibodies of the Invention

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 51 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12-14 respectively, and wherein the VL when paired with a heavy chain variable region (VH) comprising the amino acid sequence set forth in SEQ ID NO: 45 binds to CCR8.

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 15-17, respectively, and wherein the VH when paired with a light chain variable region (VL) comprising the amino acid sequence set forth in SEQ ID NO: 52 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 13 and 20, respectively, and wherein the VL when paired with a heavy chain variable region (VH) comprising the amino acid sequence set forth in SEQ ID NO: 46 binds to CCR8.

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 21, 16 and 23, respectively, and wherein the VH when paired with a light chain variable region (VL) comprising the amino acid sequence set forth in SEQ ID NO: 51 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 13 and 20, respectively, and wherein the VL when paired with a heavy chain variable region (VH) comprising the amino acid sequence set forth in SEQ ID NO: 45 binds to CCR8.

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 27-29, respectively, and wherein the VH when paired with a light chain variable region (VL) comprising the amino acid sequence set forth in SEQ ID NO: 54 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 30-32, respectively, and wherein the VL when paired with a heavy chain variable region (VH) comprising the amino acid sequence set forth in SEQ ID NO: 48 binds to CCR8.

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 55 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 12, 37 and 14, respectively, and wherein the VL when paired with a heavy chain variable region (VH) comprising the amino acid sequence set forth in SEQ ID NO: 45 binds to CCR8.

The present invention also provides a polynucleotide including a polynucleotide encoding a polypeptide comprising an immunoglobulin heavy chain or a fragment thereof comprising a heavy chain variable region (VH) comprising complementarity determining regions (CDRs) 1, 2, and 3 with the amino acid sequences set forth in SEQ ID NOs: 9-11, respectively, and wherein the VH when paired with a light chain variable region (VL) comprising the amino acid sequence set forth in SEQ ID NO: 56 binds to CCR8.

The present invention also provides a polynucleotide including a polynucleotide encoding a polypeptide comprising an immunoglobulin light chain or a fragment thereof comprising a light chain variable region (VL) comprising complementarity determining regions (CDRs) 1, 2, and 3 with the amino acid sequences set forth in SEQ ID NOs: 42, 13 and 14, respectively, and wherein the VL when paired with a heavy chain variable region (VH) comprising the amino acid sequence set forth in SEQ ID NO: 45 binds to CCR8.

In some embodiments, the VH when paired with a VL specifically binds to human CCR8 and/or Cynomolgus CCR8, and the VL when paired with a VH specifically binds to human CCR8 and/or Cynomolgus CCR8.

The present invention also provides a polynucleotide comprising a polynucleotide encoding a polypeptide comprising an immunoglobulin light chain comprising the amino acid sequence set forth in SEQ ID NO: 76, and wherein the light chain when paired with a heavy chain comprising the amino acid sequence set forth in SEQ ID NO: 74 binds to CCR8.

The present invention also provides a polynucleotide comprising a polynucleotide encoding a polypeptide comprising an immunoglobulin heavy chain comprising the amino acid sequence set forth in SEQ ID NO: 74, and wherein the heavy chain when paired with a light chain comprising the amino acid sequence set forth in SEQ ID NO: 76 binds to CCR8.

In some embodiments, the polynucleotide encoding a polypeptide comprising an immunoglobin light chain comprises the nucleotide sequence of SEQ ID NO: 79.

In some embodiments, the polynucleotide encoding a polypeptide comprising an immunoglobin light chain comprises the nucleotide sequence of SEQ ID NO: 82.

In some embodiments, the polynucleotide encoding a polypeptide comprising an immunoglobin heavy chain comprises the nucleotide sequence of SEQ ID NO: 80.

In some embodiments, the polynucleotide encoding a polypeptide comprising an immunoglobin heavy chain comprises the nucleotide sequence of SEQ ID NO: 77.

The present invention also provides a polynucleotide comprising a polynucleotide encoding a polypeptide comprising an immunoglobulin light chain comprising the amino acid sequence set forth in SEQ ID NO: 76, and wherein the light chain when paired with a heavy chain comprising the amino acid sequence set forth in SEQ ID NO: 75 binds to CCR8.

The present invention also provides a polynucleotide comprising a polynucleotide encoding a polypeptide comprising an immunoglobulin heavy chain comprising the amino acid sequence set forth in SEQ ID NO: 75, and wherein the heavy chain when paired with a light chain comprising the amino acid sequence set forth in SEQ ID NO: 76 binds to CCR8.

In some embodiments, the polynucleotide encoding a polypeptide comprising an immunoglobin light chain comprises the nucleotide sequence of SEQ ID NO: 79.

In some embodiments, the polynucleotide encoding a polypeptide comprising an immunoglobin light chain comprises the nucleotide sequence of SEQ ID NO: 82.

In some embodiments, the polynucleotide encoding a polypeptide comprising an immunoglobin heavy chain comprises the nucleotide sequence of SEQ ID NO: 81.

In some embodiments, the polynucleotide encoding a polypeptide comprising an immunoglobin heavy chain comprises the nucleotide sequence of SEQ ID NO: 78.

### III. Methods of Generating Anti-CCR8 Antibodies

As set forth herein, the present invention provides anti-C—C chemokine type 8 (CCR8) antibodies and antibody fragments thereof, methods of making the antibodies or antigen binding fragments thereof.

Chemokine receptors have traditionally been very difficult antigens to develop antibodies against. CCR8 protein was proved to be a particularly unstable protein in comparison to other multi-span GPCRs. The minimal surface exposure and flexible topology makes CCR8 a challenging antibody target. Currently, no soluble protein for immunizations, sorting or screening is available. Therefore, due to these difficulties, researchers in this field have had a low success rate in developing antibodies to CCR8.

The present inventors, however, have successfully developed a unique and superior approach for generating antibodies targeting the specific chemokine receptor CCR8. Specifically, the inventors first developed a CCR8 mutagenesis screen in which each residue in the transmembrane and the intracellular regions of CCR8 were substituted with all 19 non-wild type amino acids in order to identify stabilizing CCR8 mutants. About 2000 unique sequences were screened for beneficial mutations, and a particular mutant with 11 amino acid substitutions was identified to improve stability while maintaining natural ligand binding capabilities of CCR8. Subsequently, the identified CCR8 mutant is presented in a nanodisc as a soluble antigen, and used as an immunogen for antibody production. Using this approach, the inventors had successfully identified a number of anti-CCR8 antibodies, as described herein.

Therefore, in one aspect, the present invention provides a method of generating an antibody or antigen-binding fragment thereof that bind specifically to human CCR8 protein. The method comprises preparing a soluble CCR8 by presenting the CCR8 protein in a synthetic membrane system; wherein the CCR8 protein is a mutant form of CCR8, and generating antibodies or antigen-binding fragment thereof against the soluble CCR8.

In some embodiments, the CCR8 protein comprises one or more mutations in the intracellular region and/or the transmembrane domain.

In some embodiments, the synthetic membrane system comprises a nanodisc composed of a phospholipid bilayer encircled by two copies of a membrane scaffold protein.

Additional methods can be used for obtaining antibodies, or antigen binding fragments thereof, of the present invention. For example, antibodies, and antigen-binding fragments thereof, can be produced using recombinant DNA methods. Expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments thereof or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments thereof. Furthermore, transgenic animals, preferably mammals, comprising the aforementioned host cells may be used for the large scale production of the antibody or antibody fragments thereof.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments thereof, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments thereof can then be isolated from the growth medium, cellular lysates, or cellular membrane fractions. The isolation and purification of the, e.g., microbially expressed antibodies or antigen binding fragments thereof may be by any conventional means such as, for example, preparative chromatographic separations and immunological separations such as those involving the use of monoclonal or polyclonal antibodies directed, e.g., against the constant region of the antibody.

Aspects of the present invention relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies, and antigen-binding portions thereof.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment thereof may be undertaken by culturing a modified recombinant host under culture conditions appropriate for the growth of the host cells and the expression of the coding sequences. The antibodies or antigen binding fragments thereof may be recovered by isolating them from the culture. The expression systems may be designed to include signal peptides so that the resulting antibodies are secreted into the medium; however, intracellular production is also possible.

The present invention also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments thereof may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, a polynucleotide(s) may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment thereof; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment thereof). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

Monoclonal antibodies, and antigen-binding fragments thereof, may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody, or an antigen-binding portion thereof, that specifically binds to a specified antigen, e.g., CCR8, e.g., wild type CCR8, mutant CCR8, e.g., presented in a nanodisc. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies, and antigen-binding portions thereof, includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597 WO92/18619; WO 91/17271; WO 92/20791; WO 92/15679; WO 93/01288; WO 92/01047; WO 92/09690; and WO 90/02809.

In addition to the use of display libraries, the specified antigen (e.g., CCR8) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397.

For additional antibody production techniques, see Antibodies: A Laboratory Manual, eds. Harlow et al., Cold Spring Harbor Laboratory, 1988. The present present invention is not necessarily limited to any particular source, method of production, or other special characteristics of an antibody.

Methods for generating human antibodies in transgenic mice are also known in the art. Any such known methods can be used in the context of the present invention to make human antibodies that specifically bind to human CCR8.

Using VELOCIMMUNE™ technology (see, for example, U.S. Pat. No. 6,596,541, Regeneron Pharmaceuticals, VELOCIMMUNE®) or any other known method for generating monoclonal antibodies, high affinity chimeric antibodies to human CCR8 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified lgG1 or lgG4. While the constant region selected may vary according to specific use, high affinity antigen-binding and target specificity characteristics reside in the variable region.

### IV. Therapeutic Compositions and Methods

Methods of Treating Cancer

The present present invention is based, at least in part, on the development of engineered anti-CCR8 antibodies that have an enhanced ADCC activity. The inventors have successfully demonstrated in the working examples that treatment with the anti-CCR8 antibodies of the present invention can selectively deplete intratumoral or tumor infiltrating Treg cells while having no effect on peripheral Treg cells. As a result, treatment with the anti-CCR8 antibodies of the present invention results in a selective depletion of tumor infiltrating Treg cells, and a significant reduction in tumor size and/or tumor growth in mouse tumor models. In addition, the present inventors have demonstated that treatment with the anti-CCR8 antibodies promotes the development of an antigen-specific memory response.

Accordingly, in one aspect, the present invention provides a method for treating cancer in a subject by administrating to the subject an effective amount of an anti-CCR8 antibody and antigen-binding portions thereof, as described herein.

Any type of cancer may be treated using the compositions and methods disclosed herein. In some embodiments, the cancer comprises a solid tumor cancer. In other embodiments, the cancer cancer comprises a blood-based cancer, e.g., leukemia, lymphoma, e.g., T cell lymphoma, or myeloma.

Examples of cancers that may be treated using the compositions and methods disclosed herein include, but are not limited to squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, leukemia, lymphoma, myeloma, melanoma, and various types of head and neck cancer. In some embodiments, lung cancer is non-small cell lung cancer or lung squamous cell carcinoma. In some embodiments, leukemia is acute myeloid leukemia or chronic lymphocytic leukemia. In some embodiments, lymphoma is T cell lymphoma. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma. In some embodiments, the cancer is selected from bladder cancer, cervical cancer (such as squamous cell cervical cancer), head and neck squamous cell carcinoma, rectal adenocarcinoma, non-small cell lung cancer, endometrial cancer, prostate adenocarcinoma, colon cancer, ovarian cancer (such as serous epithelial ovarian cancer), and melanoma. In one particular embodiment, the cancer is T cell lymphoma.

In another aspect, the present invention provides a method for inhibiting or reducing tumor growth in a subject by administering an effective amount of an anti-CCR8 antibody or antigen-binding portions thereof, as described herein.

In some embodiments, administration of the anti-CCR8 antibodies results in at least 5%, 10%, 15%, 20%, 25%, 30%, 35%, 40%, 45%, 50%, 55%, 60%, 65%, 70%, 75%, 80%, 85%, 90%, or 95% reduction in tumor volume. In some embodiments, administration of the anti-CCR8 antibodies results complete regression of tumor.

In another aspect, the present invention provides a method for reducing tumor infiltrating Treg cells in a subject by administering an effective amount of an anti-CCR8 antibody or antigen-binding portions thereof, as described herein.

In some embodiments, administration of the anti-CCR8 antibodies results in at least 5%, 10%, 15%, 20%, 25%, 30%, 35%, 40%, 45%, 50%, 55%, 60%, 65%, 70%, 75%, 80%, 85%, 90%, or 95% reduction in tumor infiltrating Treg cells. In some embodiments, administration of the anti-CCR8 antibodies results in complete removal of tumor infiltrating Treg cells. In some embodiments, the antibodies have no effect on peripheral Treg cells.

In another aspect, the present invention provides a method for inducing antigen-specific memory response in a subject by administering an effective amount of an anti-CCR8 antibody or antigen-binding portions thereof, as described herein.

In some embodiments, the anti-CCR8 antibodies may be administered with one or more chemotherapeutic agents, as described in detail below.

The antibodies or antigen binding portions thereof preferably are capable of binding human CCR8 both in vivo and in vitro. Accordingly, such antibodies or antigen binding portions thereof can be used to bind hCCR8, e.g., in a cell culture containing hCCR8, in human subjects or in other mammalian subjects having CCR8 with which an antibody disclosed herein cross-reacts.

Preferably, the subject is a human subject. Alternatively, the subject can be a mammal expressing a CCR8 to which antibodies of the present invention are capable of binding. Still further the subject can be a mammal into which CCR8 has been introduced (e.g., by administration of CCR8 or by expression of a CCR8 transgene). Antibodies of the present invention can be administered to a human subject for therapeutic purposes. Moreover, antibodies of the present invention can be administered to a non-human mammal expressing a CCR8 with which the antibody is capable of binding for veterinary purposes or as an animal model of human disease. Regarding the latter, such animal models may be useful for evaluating the therapeutic efficacy of antibodies of the present invention (e.g., testing of dosages and time courses of administration).

Routes of Administration, Carriers, and Dosages

In various embodiments, antibodies may be administered in vivo by various routes, including, but not limited to, oral, intra-arterial, parenteral, intranasal, intravenous, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising antibodies and other polypeptides are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising antibodies and other polypeptides may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical packs and kits comprising one or more containers, each containing one or more doses of an antibody or combinations of antibodies are also provided. In some embodiments, a unit dosage is provided wherein the unit dosage contains a predetermined amount of a composition comprising an antibody or combination of antibodies, with or without one or more additional agents. In some embodiments, such a unit dosage is supplied in single-use prefilled syringe for injection, for example, or as a kit. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment of the specific indication. The therapeutically effective amount is typically dependent on the weight of the subject being treated, his or her physical or health condition, the extensiveness of the condition to be treated, or the age of the subject being treated.

In some embodiments, an anti-CCR8 antibody is administered at a dose of 0.3 to 10 mg/kg, 0.5 to 10 mg/kg, 0.5 to 5 mg/kg, or 1 to 5 mg/kg body weight, such as at 0.3, 0.5, 1, 2, 3, 4, 5, or 10 mg/kg. In some embodiments, an anti-CCR8 antibody may be administered every week, every 2 weeks, every 3 weeks, or every 4 weeks. In some embodiments, an anti-CCR8 antibody may be administered at 1, 2, 3, or 4 mg/kg every 2 weeks. In some such embodiments, an anti-CCR8 antibody may be administered at 1, 2, 3, or 4 mg/kg every 2 weeks.

In certain embodiments, the dose of an anti-CCR8 antibody is a fixed dose in a pharmaceutical composition. In other embodiments, the method of the present invention can be used with a flat dose (a dose given to a patient irrespective of the body weight of the patient).

Combination with Other Therapies

Antibodies may be administered alone or with other modes of treatment. They may be provided before, substantially contemporaneously with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof.

Combinations with Immune Stimulating Agents

In some embodiments, the combination treatments herein may be further combined with at least one immune stimulating agent. The term “immune stimulating agent” as used herein refers to a molecule that stimulates the immune system by either acting as an agonist of an immune-stimulatory molecule, including a co-stimulatory molecule, or acting as an antagonist of an immune inhibitory molecule, including a co-inhibitory molecule. An immune stimulating agent may be a biologic or a small molecule compound. Examples of biologic immune stimulating agents include, but are not limited to, antibodies, antibody fragments, fragments of receptor or ligand polypeptides, for example that block receptor-ligand binding, vaccines and cytokines.

In some embodiments, the at least one immune stimulating agent comprises an agonist of an immune stimulatory molecule, including a co-stimulatory molecule, while in some embodiments, the at least one immune stimulating agent comprises an antagonist of an immune inhibitory molecule, including a co-inhibitory molecule. In some embodiments, the at least one immune stimulating agent comprises an agonist of an immune-stimulatory molecule, including a co-stimulatory molecule, found on immune cells, such as T cells. In some embodiments, the at least one immune stimulating agent comprises an antagonist of an immune inhibitory molecule, including a co-inhibitory molecule, found on immune cells, such as T cells. In some embodiments, the at least one immune stimulating agent comprises an agonist of an immune stimulatory molecule, including a co-stimulatory molecule, found on cells involved in innate immunity, such as NK cells. In some embodiments, the at least one immune stimulating agent comprises an antagonist of an immune inhibitory molecule, including a co-inhibitory molecule, found on cells involved in innate immunity, such as NK cells. In some embodiments, the combination enhances the antigen-specific T cell response in the treated subject and/or enhances the innate immunity response in the subject.

In certain embodiments, an immune stimulating agent targets a stimulatory or inhibitory molecule that is a member of the immunoglobulin super family (IgSF). For example, an immune stimulating agent may be an agent that targets (or binds specifically to) another member of the B7 family of polypeptides. An immune stimulating agent may be an agent that targets or binds to a member of the TNF family of membrane bound ligands or a co-stimulatory or co-inhibitory receptor binding specifically to a member of the TNF family. Exemplary TNF and TNFR family members that may be targeted by the immune stimulating agents herein include CD40 and CD40L, OX-40, OX-40L, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACT, APRIL, BCMA, LTPR, LIGHT, DcR3, HVEM, VEGI/TL1A, TRAMP/DR3, EDAR, EDA1, XEDAR, EDA2, TNFR1, Lymphotoxin α/TNFP, TNFR2, TNFα, LTPR, Lymphotoxin α 1β2, FAS, FASL, RELT, DR6, TROY and NGFR.

In some embodiments, an immune stimulating agent may comprise (i) an antagonist of a protein that inhibits T cell activation (e.g., immune checkpoint inhibitor) such as CTLA4 (e.g. an anti-CTLA4 antibody, e.g. YERVOY (ipilimumab) or tremelimumab), LAG-3 (e.g. an anti-LAG-3 antibody, for example, BMS-986016 (WO10/19570, WO14/08218), or IMP-731 or IMP-321 (WO08/132601, WO09/44273), TIM3, Galectin 9, CEACAM-1, BTLA, CD69, Galectin-1, TIGIT, CD113, GPR56, VISTA, B7-H3 (e.g. MGA271 (WO11/109400)), B7-H4, 2B4, CD48, GARP, PD1H, LAIR1, TIM-1, TIM-4, and ILT4 and/or may comprise (ii) an agonist of a protein that stimulates T cell activation such as B7-2, CD28, 4-1BB (CD137) (e.g. a CD137 agonist antibody such as urelumab or PF-05082566 (WO12/32433)), 4-1BBL, ICOS, ICOS-L, OX40 (e.g. an OX40 agonist antibody, for example, MEDI-6383, MEDI-6469 or MOXR0916 (RG7888; WO06/029879)), OX40L, GITRL, CD70, CD27 (e.g. an agonistic CD27 antibody such as varlilumab (CDX-1127)), CD40, CD40L, DR3 and CD28H. In some embodiments, the agonist of a protein that stimulates T cell activation is an antibody.

In some embodiments, an immune stimulating agent may comprise an agent that inhibits or is an antagonist of a cytokine that inhibits T cell activation (e.g., IL-6, IL-10, TGF-β, VEGF, and other immunosuppressive cytokines), and in some embodiments an immune stimulating agent may comprise an agent that is an agonist of a cytokine, such as IL-2, IL-7, IL-12, IL-15, IL-21 and IFNα (e.g., the cytokine itself) that stimulates T cell activation. TGF-β inhibitors include, e.g., GC1008, LY2157299, TEW7197 and IMC-TR1. In some embodiments, immune stimulating agents may comprise an antagonist of a chemokine, such as CXCR2 (e.g., MK-7123), CXCR4 (e.g. AMD3100), CCR2, or CCR4 (mogamulizumab). In some embodiments, the at least one immune stimulating agent comprises a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In some embodiments, immune stimulating agents may include antagonists of inhibitory receptors on NK cells or agonists of activating receptors on NK cells. In some embodiments, the at least one immune stimulating agent is an antagonist of KIR, e.g. the antibody lirilumab.

Immune stimulating agents may also include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Immune stimulating agents may also include certain vaccines such as mesothelin-targeting vaccines or attenuated listeria cancer vaccines, such as CRS-207.

Immune stimulating agents may also comprise agents that deplete or block Treg cells, such as agents that specifically bind to CD25.

Immune stimulating agents may also comprise agents that inhibit a metabolic enzyme such as indoleamine dioxigenase (IDO), dioxigenase, arginase, or nitric oxide synthetase. IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) and F001287.

Immune stimulating agents may also comprise agents that inhibit the formation of adenosine or inhibit the adenosine A2A receptor.

Immune stimulating agents may also comprise agents that reverse/prevent T cell anergy or exhaustion and agents that trigger an innate immune activation and/or inflammation at a tumor site.

The treatment combinations can also be further combined in a combinatorial approach that targets multiple elements of the immune pathway, such as one or more of the following: at least one agent that enhances tumor antigen presentation (e.g., dendritic cell vaccine, GM-CSF secreting cellular vaccines, CpG oligonucleotides, imiquimod); at least one agent that inhibits negative immune regulation e.g., by inhibiting CTLA4 pathway and/or depleting or blocking Treg or other immune suppressing cells; a therapy that stimulates positive immune regulation, e.g., with agonists that stimulate the CD-137 and/or OX-40 pathway and/or stimulate T cell effector function; at least one agent that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; at least one agent that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); at least one agent that inhibits a metabolic enzyme such as indoleamine dioxigenase (IDO), dioxigenase, arginase or nitric oxide synthetase; at least one agent that reverses/prevents T cell anergy or exhaustion; a therapy that triggers an innate immune activation and/or inflammation at a tumor site; administration of immune stimulatory cytokines or blocking of immuno repressive cytokines.

For example, the at least one immune stimulating agent may comprise one or more agonistic agents that ligate positive costimulatory receptors; one or more antagonists (blocking agents) that attenuate signaling through inhibitory receptors, such as antagonists that overcome distinct immune suppressive pathways within the tumor microenvironment; one or more agents that increase systemically the frequency of anti-tumor immune cells, such as T cells, deplete or inhibit Tregs (e.g., by inhibiting CD25); one or more agents that inhibit metabolic enzymes such as IDO; one or more agents that reverse/prevent T cell anergy or exhaustion; and one or more agents that trigger innate immune activation and/or inflammation at tumor sites.

Other Combination Therapies

For treatment of cancer, as discussed herein, the antibodies may be administered in conjunction with one or more additional anti-cancer agents, such as the chemotherapeutic agent, growth inhibitory agent, anti-angiogenesis agent and/or anti-neoplastic composition. Nonlimiting examples of chemotherapeutic agents, growth inhibitory agents, anti-angiogenesis agents, anti-cancer agents, and anti-neoplastic compositions that can be used in combination with the antibodies of the present invention are as follows.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, an anti-CCR8 antibody may be further administered with gemcitabine-based chemotherapy in which one or more chemotherapy agents including gemcitabine or including gemcitabine and nab-paclitaxel are administered. In some such embodiments, an anti-CCR8 antibody may be administered with at least one chemotherapy agent selected from gemcitabine, nab-paclitaxel, leukovorin (folinic acid), 5-fluorouracil (5-FU), irinotecan, and oxaliplatin. FOLFIRINOX is a chemotherapy regime comprising leukovorin, 5-FU, irinotecan (such as liposomal irinotecan injection), and oxaliplatin. In some embodiments, an an anti-CCR8 antibody may be further administered with gemcitabine-based chemotherapy. In some embodiments, the anti-CCR8 antibody may be further administered with at least one agent selected from (a) gemcitabine; (b) gemcitabine and nab-paclitaxel; and (c) FOLFIRINOX. In some embodiments, the at least one agent is gemcitabine. In some such embodiments, the cancer to be treated is pancreatic cancer.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA, or VEGF receptor(s), and other bioactive and organic chemical agents, etc. Combinations thereof are also included in the invention.

Unless otherwise defined, all technical and scientific terms used herein have the same meaning as commonly understood by one of ordinary skill in the art to which this invention be-longs. Although methods and materials similar or equivalent to those described herein can be used, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1. CCR8 Expression is Restricted to the Tumor Microenvironment

To identify cell types that express CCR8, a flow cytometry based profiling experiment was performed. Human peripheral blood mononuclear cells (PBMCs) were isolated from healthy donors by Ficoll density gradient centrifugation according to standard procedures. A combination of fresh and frozen single cell renal cell carcinoma (RCC) specimens were analyzed. Frozen dissociated tumor cells (DTCs; Conversant/Discovery Life Sciences) were thawed by addition of pre-warmed medium (RPMI 1640, 10% fetal calf serum, 55 μM β-mercaptoethanol and non-essential amino acids). Thawed DTCs were rested for 30 min at 37° C. 5% CO2 prior to staining. Fresh tumor biopsies were processed to single cell suspensions by a combination of mechanical dissociation and enzymatic digestion. Excised tissue was cut into small pieces using a scalpel and transferred to a 75 mL Erlenmeyer flask containing 10 mL of medium supplemented with 0.5 mg/mL Collagenase IV (Worthington Biochemical Corporation; cat #LS004210) and 0.01 mg/mL DNAse I (Worthington; cat #LS002058). The tissue was incubated for 20 min at 37° C. 5% CO2 on an orbital shaker prior to passage through a 70 μM filter and dissociation of any remaining tissue fragments with a syringe. The filter was washed by the addition of 20 mL of medium. Red cells were lysed by incubation with ACK lysis buffer for 2 min at RT and quenched by the addition of 10 mL of medium. Samples were passed through a 30 μM filter and resuspended in FACS buffer (PBS pH 7.2, 0.5% BSA, 2 mM EDTA and 0.09% sodium azide) for staining.

PBMC and tumor single cell suspensions were incubated with Near IR Dead Cell stain (Life Technologies; cat #L34976) for 30 min and washed twice prior to blockade of Fc-receptors for 15 min with 50 μL FACS buffer containing 50 μg/mL of each of the following: Fc receptor binding inhibitor antibody (Thermo Fisher; cat #14-9161-73), purified NA/LE Human BD Fc Block (BD Biosciences; cat #564765 and Hinge-Fc (Five Prime Therapeutics; RPN00343). Fifty microliters of primary antibody cocktail was added directly to the cells in Fc-block and samples were incubated for an additional 30 min. Samples were washed twice in FACS buffer and fixed overnight with 0.5% paraformaldehyde/FACS buffer. Antibody cocktails were made with Brilliant stain buffer (BD Biosciences; cat #566349) diluted 1:10 with FACS buffer and all incubations were performed at 4° C. Intracellular staining for FOXP3 expression was performed on the fixed samples using the FOXP3/transcription factor staining buffer set (eBioscience/Thermo Fischer Scientific; cat #00-5523-00) according to the manufacturer's instructions.

The following antibodies were used for immunophenotyping: CD45 AF700 (clone 2D1), CD25 PerCYP5.5 (clone M-A251), CD4 BV510 (clone RPA-T4), CD8 BV785 (clone RPA-T8), CD3 FITC (clone OKT3), CCR8 PE (clone 263G8), FOXP3 PeCy7 (clone 236A/E7), CCR4 BV421 (clone 291H4), CD56 BV711 (clone 5.1H11), CD19 PerCYP5.5 (clone SJ25C1) and CTLA4 PECF594 (clone BNI3).

CCR8 expression was found to be low or absent on peripheral leukocyte effector populations such as CD4+ and CD8+T, NK and B cells (FIG. 1A). In contrast, the highly related family member CCR4 was expressed on all effector subsets with an average of 44% of CD4+, 15% of CD8+, 8% of NK and 7% of B cells expressing this receptor (FIG. 1A). Intratumoral naïve CD4+ and CD8+ effector T cells do not express CCR8 to an appreciable extent whereas an average of 39% (vs 3% CCR8+) of naïve CD4+ and 15% of CD8+T (vs 0.7% CCR8+) cells express CCR4 (FIG. 1A). The majority of the CD4+CD25+ FOXP3-CTLA4− subset defined as an activated CD4+ effector population expressed CCR4 while a small proportion expressed CCR8 (average of 90 vs 27% for CCR4 vs CCR8 respectively) (FIG. 1B). In addition, CCR4 and CCR8 were highly co-expressed on this cell population. The proportion of the regulatory T cell populations that expressed CCR8 ranged from an average of 46% for CD4+CD25+ FOXP3+ CTLA4− subset to 21% for the CD4+CD25+ CTLA4+ subset (FIG. 1B). While CCR8 tended to be co-expressed with CCR4 on both activated CD4+ effector and the CD4+CD25+ FOXP3+ CTLA4− regulatory T cells, a significant number of patients exhibited differential expression of CCR8 on the CD4+CD25+ CTLA4+ subset with an average of 12% of this population exhibiting a CCR4− CCR8+ immunophenotype (FIG. 1B).

While a small proportion of activated CD4+ effector T cells expressed CCR8, most of the expression was observed on intratumoral Treg populations (FIG. 1B). The absence of CCR8 expression on human peripheral leukocyte effector populations confirmed that CCR8 expression is enriched in the microenvironment of human RCC. Critically, CCR8 is not expressed on CD8+ effector T cells that are known to be key drivers of anti-tumor immunity in humans, which is a key differentiator from the closely related family member CCR4.

### Example 2: Efficacy of Anti-Murine CCR8 Depleting Antibody in the CT26 Syngeneic Tumor Model

The CT26 murine tumor model was utilized to evaluate the impact of anti-CCR8 treatment on tumor growth. Six-eight-week-old female BALB/c mice (n=10 per group) were inoculated with 1×10⁶ CT26 colon adenocarcinoma cells and test articles administered intravenously when the tumors reached an average volume of 144 mm³. Antibodies were produced bearing either the murine wild-type, enhanced ADCC (eADCC; mutated Fc (S239D/A330L/1332E) to enhance binding to FcγRIII) or Fc-silent (mutated Fc to reduce binding to FcγRIII) IgG2a Fc isotype backbones to evaluate the requirement for ADCC-mediated depletion. The anti-mCCR8 (clone I962)-mIgG2a (eADCC and Fc-silent) and mIgG2a isotype control antibodies were dosed at 10 mg/kg on days 4 and 7 post-tumor cell inoculation. The anti-mCTLA4-mIgG2a (clone 9D9) binds to CTLA4 expressed on murine T_{regs} and was dosed at 1 mg/kg on Days 4, 7, and 11. Anti-CTLA4 served as a positive control for T_{reg} depletion-mediated tumor growth inhibition. Tumor length (L) and width (W) was measured using electronic calipers and volume (V) calculated using V=(L×W²)/2. Statistical significance was determined vs mIgG2a isotype control group using One-way ANOVA.

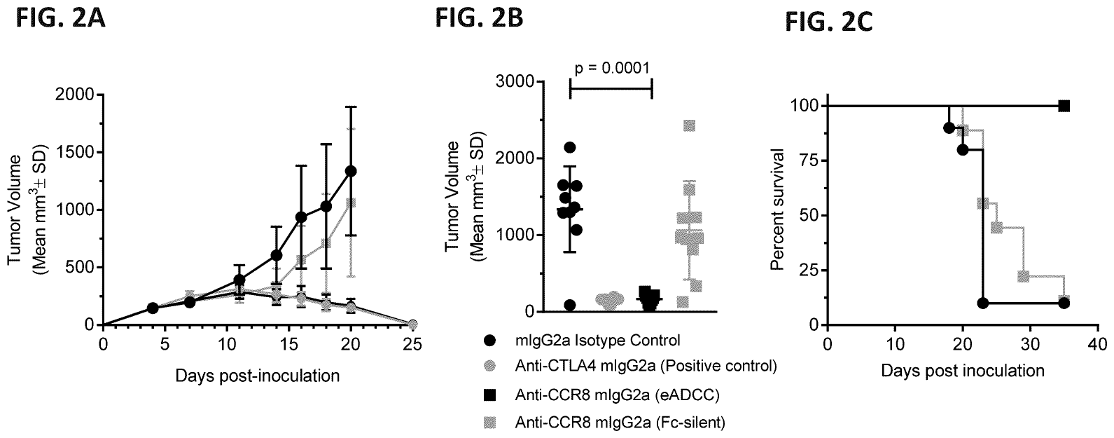

Administration of an anti-murine CCR8 depleting antibody resulted in potent, single agent activity in the CT26 model that was comparable to anti-CTLA4 treatment (FIG. 2A). Following treatment with anti-CCR8 mIgG2a (eADCC) antibody, a reduction in tumor growth was observed by Day 14 post-tumor inoculation with a greater than 80% reduction in tumor burden observed by Day 20 in comparison to mIgG2a-treated control mice (FIG. 2B). Anti-CCR8 mIgG2a (Fc-silent) treated mice exhibited comparable tumor growth kinetics to mIgG2a isotype control treated mice (FIG. 2A). Kaplan-Meier analysis demonstrated that the entire cohort treated with anti-CTLA4 mIgG2a or anti-CCR8 mIgG2a (eADCC) antibody and none of the mice treated with anti-CCR8 mIgG2a (Fc-silent) remained tumor-free until the end of the study on Day 35 (FIG. 2C). These results highlight that anti-tumor activity is dependent on intact Fc-effector function and suggest that ADCC-dependent depletion of intratumoral T_{regs} is a key mechanism of action.

### Example 3: Efficacy of Anti-Murine CCR8 Depleting Antibody in the MC38 Syngeneic Tumor Model

The impact of anti-CCR8 treatment on tumor growth was also tested in the MC38 tumor model. Six to eight-week-old female C57BL/6 mice (n=10 per group) were inoculated with 0.5×10⁶ MC38 colon adenocarcinoma cells and test articles administered intravenously when the tumors reached an average volume of 123 mm³. Antibodies were generated on either the murine wild-type, enhanced ADCC (eADCC; mutated Fc to enhance binding to FcγRIII) or Fc-silent (mutated Fc to reduce binding to FcγRIII) IgG2a Fc isotype backbones to evaluate the requirement for ADCC-mediated depletion. The anti-murine CCR8-mIgG2a (clone 1962; eADCC and Fc-silent) and mIgG2a isotype control antibodies were dosed at 10 mg/kg on days 6 and 9 post-tumor cell inoculation. The anti-mCTLA4-mIgG2a (clone 9D9) binds to CTLA4 expressed on murine T_{regs} and was dosed at 1 mg/kg on Days 6, 9, and 13. This regimen serves as a positive control for T_{reg} depletion-mediated tumor growth inhibition. Tumor length (L) and width (W) was measured using electronic calipers and volume (V) calculated using V=(L×W²)/2. For Day 23 tumor volume comparisons, statistical significance was determined vs mIgG2a isotype control group using One-Way ANOVA. Statistical significance for the Kaplan-Meier analysis was determined vs mIgG2a isotype control group using the Log-rank test. A p value of less than 0.05 was considered significant.

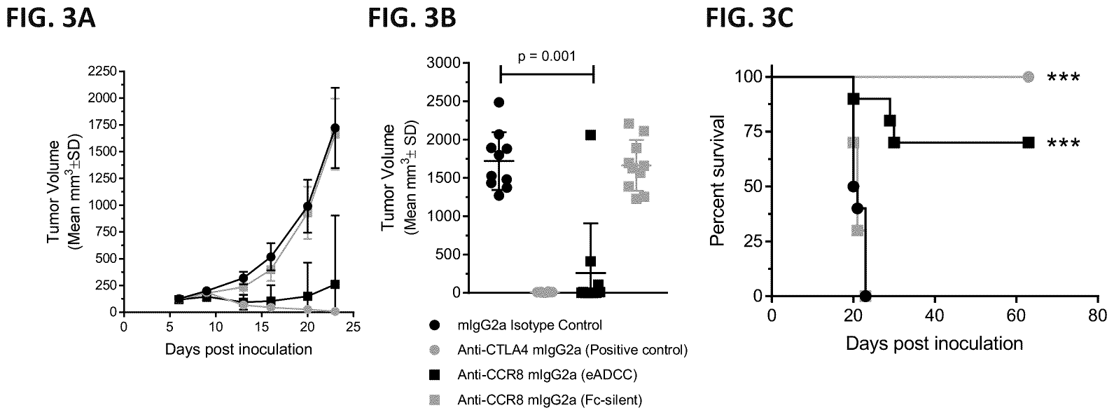

Administration of an anti-murine CCR8 depleting antibody resulted in potent, single agent anti-tumor activity in the MC38 model that was comparable to anti-CTLA4 treatment (FIG. 3A). Following treatment with anti-CCR8 mIgG2a (eADCC) antibody, a reduction in tumor growth was observed by Day 12 post-tumor inoculation with a greater than 80% reduction in tumor burden observed by Day 23 in comparison to mIgG2a-treated control mice (FIG. 3B). Anti-CCR8 mIgG2a (Fc-silent) treated mice exhibited comparable tumor growth kinetics to mIgG2a isotype control treated mice (FIG. 3A). Kaplan-Meier analysis demonstrated that the entire cohort treated with anti-CTLA4 mIgG2a antibody, seven of the ten mice treated with anti-CCR8 mIgG2a (eADCC) antibody and none of the mice treated with anti-CCR8 mIgG2a (Fc-silent) remained tumor-free until the end of the study on Day 63 (FIG. 3C). These results highlight that anti-CCR8 mediated anti-tumor activity is also dependent on Fc-effector function in the MC38 model and suggests that ADCC-dependent depletion of intratumoral T_{regs} is a likely mechanism of action.

### Example 4: Selective Depletion of Intratumoral Tregs by Treatment with an Anti-Murine CCR8 Depleting Antibody in the MC38 Syngeneic Tumor Model

Six to eight-week-old female C57BL/6 mice (n=5 per group) were inoculated with 0.5×10⁶ MC38 colon adenocarcinoma cells and test articles administered intravenously when the tumors reached an average volume of 100 mm³. Antibodies were generated on either the murine wild-type, enhanced ADCC (eADCC; mutated Fc to enhance binding to FcγRIII) or Fc-silent (mutated Fc to reduce binding to FcγRIII) IgG2a Fc isotype backbones to evaluate the requirement for ADCC-mediated depletion. Tumors were harvested on Days 3, 7 and 10 following a single dose of 10 mg/kg for analysis. Single cell suspensions were prepared using a commercial enzyme mix (Miltenyi cat #130-096-730) according to the manufacturer's instructions and mechanical dissociation using the GentleMACS (Miltenyi) m_impTumor_02 program. Cell suspensions were filtered through a 70 mM filter and resuspended at 1×10⁶ cells per mL in PBS/5% FCS (FACS buffer). 1×10⁶ cells were incubated with mouse Fc-block diluted in FACS buffer with 0.09% sodium azide for 15 min prior to the addition of cell surface antibody cocktail for 45 min. Samples were washed twice and incubated with viability stain (Live/Dead Near IR; Invitrogen cat #L34976) for 20 min. All incubations were performed on ice. Samples were washed prior to overnight fixation at 4° C. with Fix/Perm buffer (eBioscience/Thermo Fisher cat #00-5523). FOXP3 intracellular staining was performed according to the manufacturer's instructions (eBioscience/Thermo Fisher cat #00-5523) and samples acquired on a LSRFortessa (BD Biosciences). The following antibodies were used for immunophenotyping; CD3 BUV395 (clone 2C11), CD8 BUV805 (clone 53-6.7), CD25 BV510 (clone PC61), CD4 FITC (clone GK1.5), FOXP3 PE-AF610 (clone FJK-16s) and CD45 AF700 (clone 30-F11).

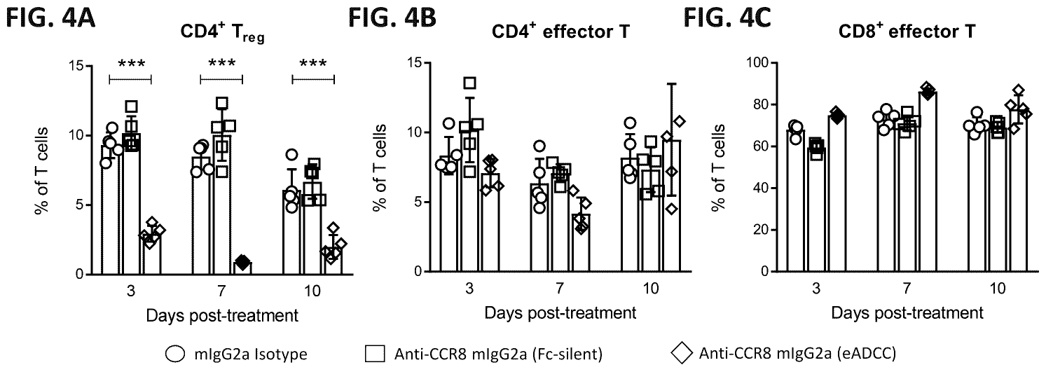

Assessment of the frequency of intratumoral Tregs as a proportion of the total CD3+ T cell population revealed a significant reduction on Day 3 following a single dose of anti-CCR8 mIgG2a (eADCC) antibody (FIG. 4A). This reduction was most significant on Day 7 and persisted until Day 10 (FIG. 4A). Treatment with either an mIgG2a isotype control or anti-CCR8 mIgG2a (Fc-silent) antibody did not alter the proportion of intratumoral Tregs at any time-point analyzed (FIG. 4A). No significant change in the proportion of intratumoral effector CD4⁺ or CD8⁺ T cells was observed following treatment with anti-CCR8 mIgG2a (eADCC) at all time-points analyzed (FIG. 4B, 4C).

The selective reduction of the intratumoral Treg population is consistent with the restricted expression of CCR8 on both mouse and human intratumoral Tregs. Consistent with the requirement for Fc-effector function to promote anti-tumor activity, the reduction of intratumoral Tregs was only observed with the eADCC and not the Fc-silent format of an anti-CCR8 mIgG2a antibody (FIG. 4A). This indicates that ADCC-dependent depletion of intratumoral T_{regs} is the primary mechanism whereby an anti-CCR8 depleting antibody induces anti-tumor activity.

### Example 5: Selective Depletion of Murine Intratumoral but not Peripheral Tregs

Six to eight-week-old female C57BL/6 mice (n=5 per group) were inoculated with 0.5×10⁶ MC38 colon adenocarcinoma cells and test articles administered intravenously when the tumor reached an average volume of 96 mm³. The anti-murine CCR8-(clone I962)-mIgG2a (eADCC) and mIgG2a isotype control antibodies were dosed once at 3 mg/kg and tumor, spleens and peripheral blood harvested 3 days post-treatment (10 days post-tumor cell inoculation) for analysis. Single-cell suspensions were prepared from tumors by enzymatic digestion and from spleens by mechanical dissociation. Staining was performed according to standard methods. Statistical significance was determined by unpaired Student's t-test and p values less than 0.05 considered significant.

The following antibodies were used for immunophenotyping: CD3 BUV395 (clone 145-2C11), CD8 BUV805 (clone 53-6.7), CTLA4 BV421 (clone UC10-4B9), CD25 BV510 (clone PC61), CD4 FITC (clone GK1.5), FOXP3 PE Texas Red (clone FJK-16s) and CD45 AF700 (clone 30-F11).

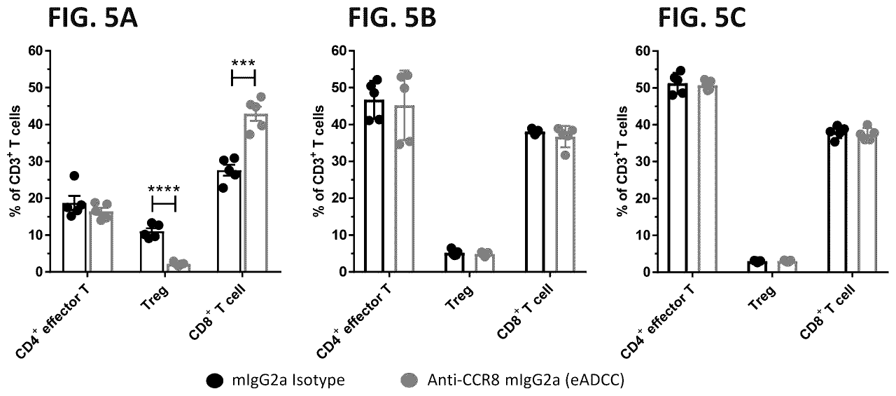

Consistent with a reduction in tumor volume and compared to the mIgG2a isotype control-treated cohort, the frequency of intratumoral CD3⁺ CD4⁺ CD25⁺ FOXP3⁺ Tregs was significantly reduced by treatment with an anti-mCCR8 mIgG2a (eADCC) antibody (FIG. 5A). The average proportion of intratumoral Treg (as a percentage of total CD3⁺ T cells) for the isotype control-treated cohort was 11% vs 2.2% for the anti-mCCR8 mIgG2a (eADCC)-treated cohort, which represents a 5-fold difference (FIG. 5A; p<0.0001). The reduction in intratumoral Treg frequency coincided with a significant increase in effector CD8⁺ T cell frequency (FIG. 5A; 28 vs 43% for isotype vs eADCC respectively; p=0.0002). In contrast, no changes in any T cell subset was observed in the spleen or peripheral blood following anti-mCCR8 mIgG2a (eADCC) treatment (FIG. 5B, 5C). This data confirmed that the depleting activity of an anti-CCR8 targeting antibody is restricted to the tumor microenvironment in a syngeneic mouse tumor model. Based on the tumor-restricted expression of CCR8 in human renal cell carcinoma, it is anticipated that a similar specificity will be observed in humans.

### Example 6: Treatment with an Anti-Murine CCR8 Depleting Antibody Promotes the Development of an Antigen-Specific Memory Response

CT26-bearing BALB/c mice were treated with single or multiple doses of either 10, 3 or 1 mg/kg anti-CCR8 mIgG2a (eADCC). These regimens resulted in complete tumor regressions in all cohorts and these complete regressor mice were pooled for subsequent tumor rechallenge approximately 12 weeks after dosing was initiated. Complete regressor or age-matched naïve BALB/c mice were inoculated with 1 or 5×10⁶ CT26 colon adenocarcinoma or 1×10⁶ EMT6 mammary adenocarcinoma cells and tumor growth monitored until the termination of the study on Day 20 post-tumor cell inoculation. Tumor length (L) and width (W) was measured using electronic calipers and volume (V) calculated using V=(L×W²)/2. For Day 20 tumor volume comparisons, statistical significance was determined using unpaired Student's t-test and p values less than 0.05 considered significant.

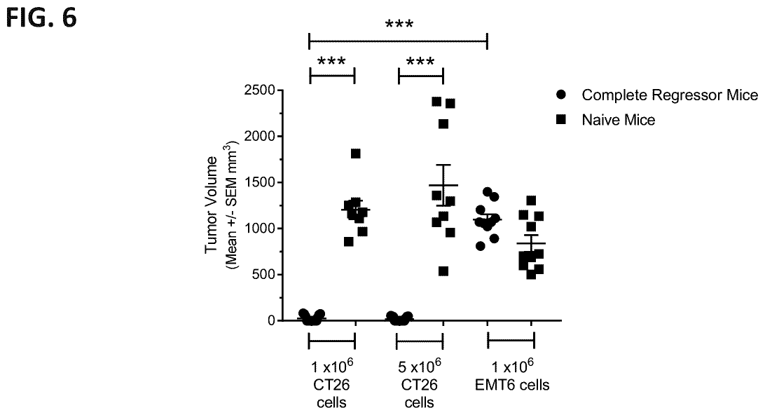

Naïve mice not previously exposed to either CT26 or EMT6 do not have tumor-specific memory responses and therefore were unable to suppress the growth of either tumor (FIG. 6). Complete regressor mice previously exposed to CT26 tumors suppressed the growth of CT26 but not EMT6 tumors, even when inoculated at a five-fold higher concentration of cells (FIG. 6). As EMT6 tumors do not share any antigens with CT26, this indicates that an antigen-specific memory response can be generated by treatment with an anti-CCR8 depleting antibody.

### Example 7: Efficacy of an Anti-Murine CCR8 Depleting Antibody in MC38 Tumor-Bearing Humanized FcγR Mice

Female humanized FcγR mice (n=8 per group; Charles River Hollister; Smith P, et al., Proceedings of the National Academy of Sciences. 2012; 109:6181-6186.) were inoculated subcutaneously with 0.5×10⁶ MC38 colon adenocarcinoma cells and test articles administered intravenously when the tumors reached an average volume of 100 mm³. Anti-murine CCR8 antibodies were generated on either a human IgG1 wild-type or enhanced ADCC (eADCC; mutated Fc (S239D/A330L/I332E) to enhance binding to FcγRIII) Fc isotype backbone to evaluate whether enhanced ADCC activity is required for anti-tumor efficacy. Anti-murine CCR8 antibodies were dosed at 3 or 0.3 mg/kg and a human IgG1 isotype control antibody at 3 mg/kg on Day 6 post-tumor cell inoculation. The anti-PD1-(clone RMP1-14)-mIgG2a (Fc-silent) binds to murine PD1⁺ cells and was dosed at 5 mg/kg on Day 6. This treatment serves as a positive control for the response of MC38 cells in humanized mice. Tumor length (L) and width (W) was measured using electronic calipers and volume (V) calculated using V=(L×W²)/2. Statistical significance was determined vs using unpaired Student's t-test and p values less than 0.05 considered significant.

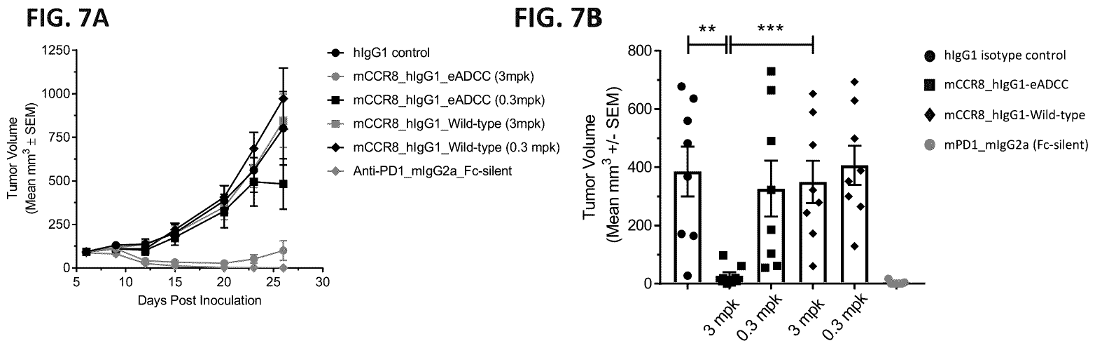

Tumor growth reduction was observed following treatment with 3 mg/kg of the ADCC-enhanced human IgG1 format of an anti-murine CCR8 antibody (FIG. 7A). On Day hIgG1 isotype control-treated mice exhibited an average tumor volume of 386 mm³, whereas anti-mCCR8 hIgG1 (eADCC)-treated mice exhibited an average volume of 28 mm³ (FIG. 7B; p=0.001). No activity was observed when the dose of anti-mCCR8 hIgG1 (eADCC) antibody was reduced to 0.3 mg/kg (FIG. 7A). Mice treated with the anti-mCCR8 hIgG1 (Wild-type) format failed to reduce tumor growth at any dose tested (FIG. 7A; mean tumor volume on Day 20=350 mm³; p=0.007 vs mCCR8 hIgG1 (eADCC)-treated cohort). The reduction of tumor growth observed with the anti-PD1 positive control antibody confirmed that MC38 tumors retained sensitivity to therapy when grown in humanized FcγR mice (FIG. 7A). This data confirmed that an ADCC enhanced format of an anti-human CCR8 depleting antibody is required for optimal anti-tumor activity.

### Example 8: Identification of Human and Cynomologus Monkey Cross-Reactive Anti-CCR8 Antibodies

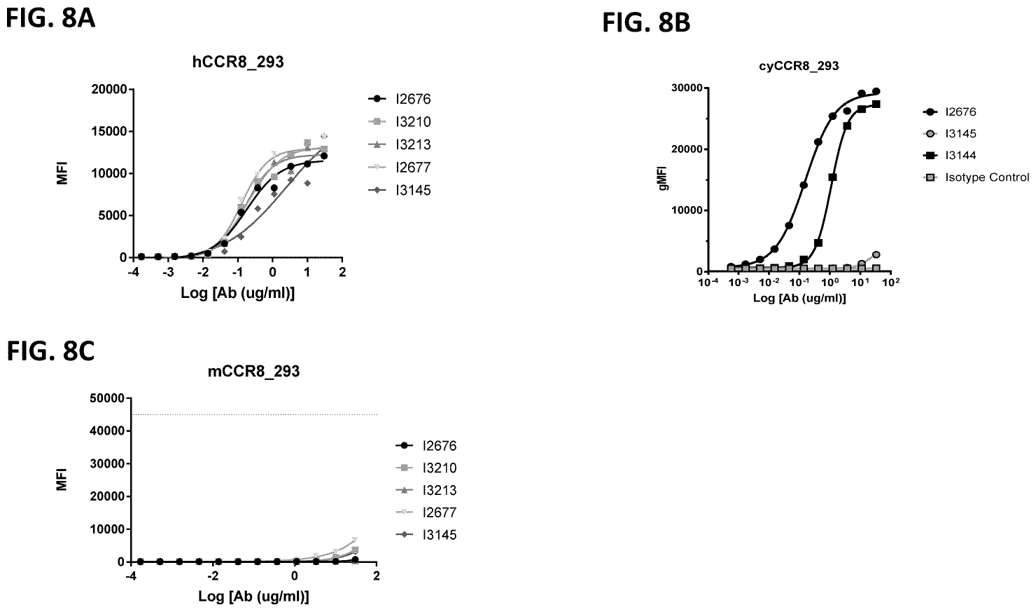

To determine the binding potency of anti-CCR8 antibodies, a flow based binding study was performed. HEK293 were transfected using expression vectors containing human CCR8 (hCCR8), cynomolgus CCR8 (cyCCR8) or murine CCR8 (mCCR8). Anti-CCR8 antibodies were tittered in the presence of hCCR8_293 (FIG. 8A), cyCCR8_293 cells (FIG. 8B) or mCCR8_293 cells (FIG. 8C). The cells were subsequently stained with an anti-hIgG secondary antibody conjugated to Brilliant Violet 421 (Biolegend) to visualize bound anti-CCR8 antibodies. The geometric mean fluorescence intensity of each sample was used to determine the binding EC50 of each antibody with Prism v.8 (Table 4; Graphpad).

TABLE 4
EC50 binding values for HEK293 cells.

| | Human CCR8 | | Cyno CCR8 | | Mouse CCR8 | |
|---|---|---|---|---|---|---|
| Clone | μg/mL | nM | μg/mL | nM | μg/mL | nM |
| I2676 | 0.18 | 1.2 | 0.30 | 2.0 | N.A. | N.A. |
| I2677 | 0.13 | 0.9 | 9.6 | 64.0 | N.A. | N.A. |
| I3144 | 0.69 | 4.6 | 37.1 | 247.3 | N.A. | N.A. |
| I3145 | 2.43 | 16.2 | 0.99 | 6.6 | N.A. | N.A. |
| I3210 | 0.20 | 1.3 | N.D. | N.D. | N.A. | N.A. |
| I3213 | 0.16 | 1.1 | N.D. | N.D. | N.A. | N.A. |

### Example 9: Identification of hCCR8-Specific Antibodies

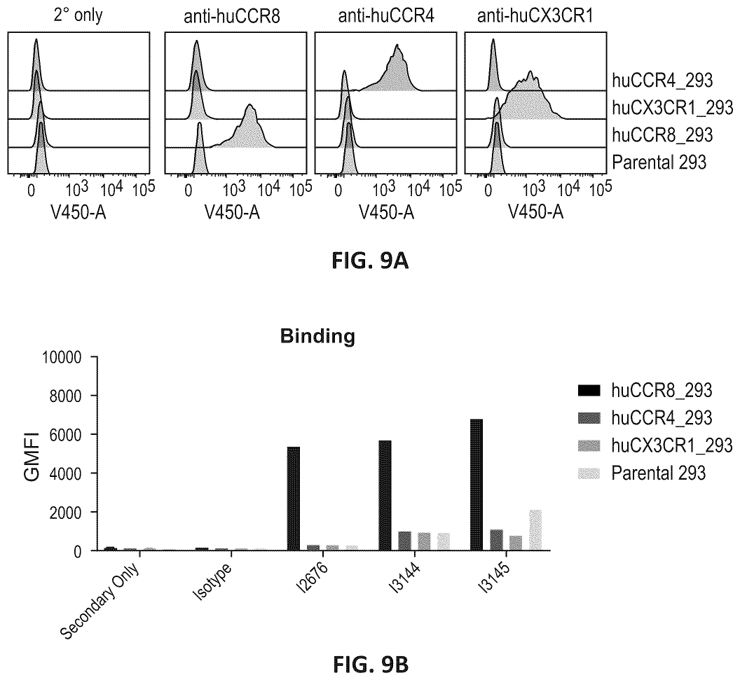

CCR4 and CX3CR1 are the most closely related proteins to CCR8 based on analysis of protein sequence homology (Pharmacological Reviews January 2014, 66 (1) 1-79). To determine the specificity of anti-CCR8 antibodies, a flow cytometry based binding study was performed. HEK293 were transfected using expression vectors containing hCCR8, human CCR4 (hCCR4) or human CX3CR1 (hCX3CR1). Expression of CCR8, CCR4 or CX3CR1 was confirmed on each of the lines by flow cytometry. Accordingly, each of the lines were stained with an unlabeled anti-hCCR8 (Clone L263G8; BioLegend), anti-hCCR4 (Clone L291H4; BioLegend) or anti-hCX3CR1 (Clone K0124E1; BioLegend) antibody. Cells were subsequently stained with an anti-mouse IgG antibody conjugated to Brilliant Violet 421 (Biolegend) to visualize bound antibodies (FIG. 9A).

To assess antibody binding selectivity, the cell lines were first labelled with unique combinations of fluorescent dyes to permit antibody staining of all cell lines in a single tube. hCCR8_293 cells were labeled with Cell Trace Far Red (Invitrogen). hCCR4_293 cells were labeled with carboxyfluorescein succinimidyl ester (Invitrogen). hCX3CR1_293 cells were labeled with both Cell Trace Far Red and carboxyfluorescein succinimidyl ester. Parental 293 cells were not labeled with dye. Anti-CCR8 antibodies were prepared at a concentration of 10 ug/ml and incubated with labeled hCCR8_293, hCCR4_293, hCX3CR1_293 or parental 293 cells that were pre-mixed at an equivalent ratio. The cells were washed and then subsequently stained with an anti-hIgG secondary antibody conjugated to Brilliant Violet 421 (Biolegend) to visualize bound antibodies. To determine the selectivity of binding, the individual cell lines were first identified in each sample and the geometric median fluorescence intensity of the secondary antibody was obtained for each population (FIG. 9B). Antibodies that yielded signal on individual transfectant lines that were greater than that observed on the parental 293 line were characterized as having specific binding for the transfectant line. Table 5 provides a summary of the binding characteristics of the antibodies that were investigated.

TABLE 5
Antibody Selectivity Profile

| Clone | CCR8 | CCR4 | CX3CR1 |
|---|---|---|---|
| I2676 | + | — | — |
| I2677 | + | — | — |
| I3144 | + | — | — |
| I3145 | + | — | — |
| I3210 | + | ND | ND |
| I3213 | + | ND | ND |

### Example 10: Establishing the ADCC Activity of Anti-CCR8 Antibodies

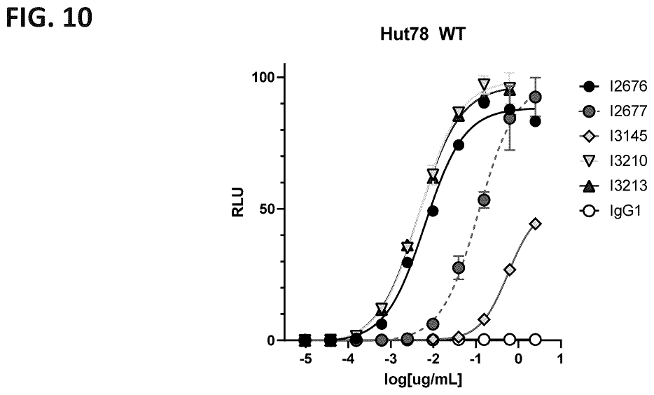

To determine the capacity of anti-CCR8 antibodies to induce ADCC, a FcR-activation reporter cell assay was performed. Anti-CCR8 antibodies were titrated in the presence of CCR8-expressing Hut78 cells and then Jurkat cells expressing FcγRIIIa and a luciferase gene under the control of the NFAT promoter (Promega). Following incubation, luciferase substrate was added, and luminescence was measured by an EnVision plate reader (Perkin Elmer). Luminescence was normalized to the maximal signal per plate, graphed as relative luminescence units (RLU) and used to determine the EC50 for each antibody with Prism v.8 (FIG. 10 and Table 6, Graphpad).

TABLE 6
FcR reporter activation - EC values in Hut78 target cells

EC50

| Antibody | μg/mL | pM |
|---|---|---|
| I2676 | 0.005915 | 39.43 |
| I2677 | 0.1158 | 772.1 |
| I3144 | 0.0091 | 60.77 |
| I3145 | 0.5686 | 3791 |
| I3210 | 0.004819 | 32.13 |
| I3213 | 0.004552 | 30.35 |

### Example 11: Anti-CCR8 Antibodies Elicit NK Cell Mediated Killing of CCR8 Expressing Target Cells

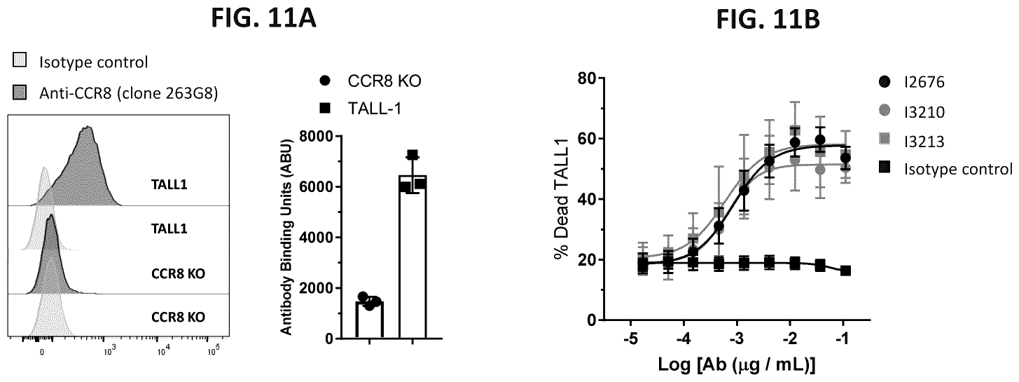

CCR8 receptor levels were quantitated on the TALL1 cell line using Quantum Simply Cellular Rat IgG microspheres (FIG. 11A; BANGs Laboratories; cat #817). Briefly, a saturating amount of anti-CCR8 (clone L263G8) was incubated with 1 drop of each microsphere standard or 250 000 TALL1 cells in a total volume of 100 μL for 30 min at 4° C. Samples were washed ×3 with 1 mL FACS buffer (PBS pH 7.2, 0.5% BSA, 2 mM EDTA and sodium azide) and resuspended in 200 μL FACS buffer for acquisition using an LSR II (BD Biosciences).

Peripheral blood mononuclear cells (PBMCs) were isolated from healthy donors using Ficoll density gradient centrifugation according to standard procedures. Primary human NK cells were purified from PBMCs using negative selection and according to the manufacturer's instructions (Miltenyi Biotec; cat #130-092-657). Purified NK cells were cultured overnight in R10 medium (RPMI with L-Glut and HEPES; Corning cat #10-041-CM, 10% heat-inactivated fetal calf serum, xl Penicillin/Streptomycin, non-essential amino acids and sodium pyruvate) prior to use in the killing assay.

Antibody dilutions were prepared at a ×2 final concentration in R10 medium starting at a top concentration of 2 μg/mL concentration and diluted 1:3 for a total of 11-points. Twenty-five microliters containing 30 000 purified NK cells and 25 μL containing 10 000 target cells were dispensed per well of a 96-well U-bottom TC-treated plate. Fifty microliters of diluted antibody was added per well, mixed and plates centrifuged for 2 minutes at 50 g prior to incubation for 4 hours at 37° C. 5% CO₂. To assess cell death, samples were washed ×1 with PBS and resuspended in Live/Dead Near IR solution (Thermo Fisher; cat #L34976) for 10 min at RT. Samples were washed twice with FACS buffer and fixed overnight in 0.5% paraformaldehyde/FACS prior to acquisition.

Target cells were distinguished from NK cells using forward and side scatter parameters and the percentage of dead target cells determined. Values were entered into the GraphPad Prism analysis software, transformed and EC50 values (1 μg/mL) derived from a sigmoidal, 4PL curve fit of the data (FIG. 11B, Table 7).

TABLE 7
NK Killing EC50 values

| Antibody | EC50 (μg/mL) | EC50 (pM) |
|---|---|---|
| I2676 | 0.0008565 | 6 |
| I3210 | 0.0006496 | 4.5 |
| I3213 | 0.000602 | 4.2 |

### Example 12. Generation of Anti-CCR8 Antibodies

Antibodies against CCR8 were generated using a human antibody phage display library XOMA050 expressing Fab fragments. The target used for the library panning was a mutein of CCR8 with a GFP tag embedded in nanodiscs (Dang et al., Nature; 552(7685):426-429 (2017)). The nanodiscs were biotinylated at their membrane scaffold protein and biotinylated CCR8 nanodiscs were used in a soluble panning approach.

Selection of target specific antibody from phage display was carried out according to methods described by Dominik et al. (Methods Enzymol.; 557:219-45 (2015)). Briefly, the phage display library was cleared with empty nanodiscs and biotinlyated GFP presented on streptavidin coated beads (DYNABEADS® M-280 Streptavidin, Invitrogen). The subtracted library was then panned against biotinylated CCR8 nanodiscs displayed at 800 nM on streptavidin coated beads for 1 hr at RT in the presence of a 20 fold molar excess of competitors (non-biotinylated empty nanodiscs and non-biotinylated GFP). Non-specific phage particles were removed by washing the beads with wash buffer (PBS). Bound phage particles were eluted with 0.5 ml of 100 nM glycine-HCl pH 2.3 and immediately neutralized by addition of an equal volume of 1M Tris-HCl pH 7.4. Eluted phage pool was used to infect TG1 E. coli cells growing in logarithmic phase, and phagemid was rescued as described (Dominik et al., Methods Enzymol.; 557:219-45 (2015)). Selection was repeated for a total of three rounds with decreasing amounts of CCR8 nanodiscs. Single colonies obtained from TG1 cells infected with eluted phage from the third round of panning were screened for binding activity in a PPE FACS assay.

PPE FACS for Selection of Positive Clones

Briefly, single colonies obtained from the TG1 cell infected with eluted phage were used to inoculate media in 96-well plates. Microcultures were grown to an OD600=0.4-0.5 at which point expression of soluble antibody fragment was induced by addition of 1 mM IPTG following overnight culture in a shaker incubator at 30° C. Bacteria were spun down and periplasmic extract was prepared and used to detect antibody binding activity to HEK 293 cells stably overexpressing human CCR8. Untransfected HEK 293 cell lines served as negative controls. Cells were resuspended and centrifuged at 500 g for 5 minutes at 4° C. Untransfected HEK 293 were dyed with CellTrace Far Red at 1:100 according to manufacturer's suggestion and pooled with HEK293 overexpressing human CCR8. Media were aspirated and cells were resuspended in cell staining buffer (Biolegend, San Diego, CA) at 4° C. Cells were centrifuged as previously described, media were aspirated, and cells were resuspended in cell staining buffer at 4° C. to a final concentration of 2×106 cells/ml. Cells were then added to 96-well round bottom plates at 50 μl/well. 100 μl of periplasmic extract neat was added to each well so that each antibody was incubated with HEK 293 cell line stably expressing human CCR8 and the dyed negative control untransfected HEK 293 cell line. The plates were incubated on ice for 30 minutes and centrifuged as previously described. Diluted antibodies were aspirated and each well was resuspended in 200 μl of cell staining buffer at 4° C. The plates were centrifuged as previously described, and the cell staining buffer was aspirated. After this washing step, cells in each well were resuspended in 100 μl of a 1:1000 dilution of a polyclonal anti-human lambda light chain and a polyclonal anti-human kappa light chain coupled to a FITC in cell staining buffer at 4° C., and the plates were incubated in the dark on ice for 30 minutes. The plates were centrifuged as previously described, and the supernatants were aspirated. Each well was resuspended in 200 μl of cell staining buffer at 4° C., and the plates were centrifuged as previously described. The cell staining buffer was then aspirated. Cells in each well were resuspended in 25-100 μl of phosphate-buffer saline (PBS) containing 1% para-formaldehyde (cell fixing buffer) at 4° C., and FACS analysis was performed on a LSRFortessa™ or LSR-II™ (BD Biosciences, San Jose, CA).

DNA sequences of positive binders were determined and aligned based on closest germline similarity. Synthesized gene fragments were subcloned into mammalian expression plasmids containing human IgG constant region variants; and human kappa or human lambda constant regions. Expression plasmids for the paired heavy and light chains were then co-transfected in a HEK 293-based mammalian cell line for expression of the recombinant, full-length, human IgG antibodies.

Alternatively, the CCR8 protein may be presented in a liposome as a soluble antigen, and used as an immunogen for antibody production.

Using these approaches, a number of anti-CCR8 antibodies were identified, as described herein.

Informal Sequence Listing

| SEQ | | |
|---|---|---|
| ID | | |
| NO | Description | Sequence |
| 1 | Human | MDYTLDLSVTTVTDYYYPDIFSSPCDAELIQTNGKLLLAVFYCLLFVFSLLGNSLVIL |
| | CCR8 | VLVVCKKLRSITDVYLLNLALSDLLFVFSFPFQTYYLLDQWVFGTVMCKVVSGFYYIG |
| | NP_005192.1 | FYSSMFFITLMSVDRYLAVVHAVYALKVRTIRMGTTLCLAVWLTAIMATIPLLVFYQV |
| | | ASEDGVLQCYSFYNQQTLKWKIFTNFKMNILGLLIPFTIFMFCYIKILHQLKRCQNHN |
| | | KTKAIRLVLIVVIASLLFWVPFNVVLFLTSLHSMHILDGCSISQQLTYATHVTEIISF |
| | | THCCVNPVIYAFVGEKFKKHLSEIFQKSCSQIFNYLGRQMPRESCEKSSSCQQHSSRS |
| | | SSVDYIL |
| 2 | Human | GTAGTGGGAGGATACCTCCAGAGAGGCTGCTGCTCATTGAGCTGCACTCACATGAGGA |
| | CCR8 | TACAGACTTTGTGAAGAAGGAATTGGCAACACTGAAACCTCCAGAACAAAGGCTGTCA |
| | NM_005201.4 | CTAAGGTCCCGCTGCCTTGATGGATTATACACTTGACCTCAGTGTGACAACAGTGACC |
| | | GACTACTACTACCCTGATATCTTCTCAAGCCCCTGTGATGCGGAACTTATTCAGACAA |
| | | ATGGCAAGTTGCTCCTTGCTGTCTTTTATTGCCTCCTGTTTGTATTCAGTCTTCTGGG |
| | | AAACAGCCTGGTCATCCTGGTCCTTGTGGTCTGCAAGAAGCTGAGGAGCATCACAGAT |
| | | GTATACCTCTTGAACCTGGCCCTGTCTGACCTGCTTTTTGTCTTCTCCTTCCCCTTTC |
| | | AGACCTACTATCTGCTGGACCAGTGGGTGTTTGGGACTGTAATGTGCAAAGTGGTGTC |
| | | TGGCTTTTATTACATTGGCTTCTACAGCAGCATGTTTTTCATCACCCTCATGAGTGTG |
| | | GACAGGTACCTGGCTGTTGTCCATGCCGTGTATGCCCTAAAGGTGAGGACGATCAGGA |
| | | TGGGCACAACGCTGTGCCTGGCAGTATGGCTAACCGCCATTATGGCTACCATCCCATT |
| | | GCTAGTGTTTTACCAAGTGGCCTCTGAAGATGGTGTTCTACAGTGTTATTCATTTTAC |
| | | AATCAACAGACTTTGAAGTGGAAGATCTTCACCAACTTCAAAATGAACATTTTAGGCT |
| | | TGTTGATCCCATTCACCATCTTTATGTTCTGCTACATTAAAATCCTGCACCAGCTGAA |
| | | GAGGTGTCAAAACCACAACAAGACCAAGGCCATCAGGTTGGTGCTCATTGTGGTCATT |
| | | GCATCTTTACTTTTCTGGGTCCCATTCAACGTGGTTCTTTTCCTCACTTCCTTGCACA |
| | | GTATGCACATCTTGGATGGATGTAGCATAAGCCAACAGCTGACTTATGCCACCCATGT |
| | | CACAGAAATCATTTCCTTTACTCACTGCTGTGTGAACCCTGTTATCTATGCTTTTGTT |
| | | GGGGAGAAGTTCAAGAAACACCTCTCAGAAATATTTCAGAAAAGTTGCAGCCAAATCT |
| | | TCAACTACCTAGGAAGACAAATGCCTAGGGAGAGCTGTGAAAAGTCATCATCCTGCCA |
| | | GCAGCACTCCTCCCGTTCCTCCAGCGTAGACTACATTTTGTGAGGATCAATGAAGACT |
| | | AAATATAAAAAACATTTTCTTGAATGGCATGCTAGTAGCAGTGAGCAAAGGTGTGGGT |
| | | GTGAAAGGTTTCCAAAAAAAGTTCAGCATGAAGGATGCCATATATGTTGTTGCCAACA |
| | | CTTGGAACACAATGACTAAAGACATAGTTGTGCATGCCTGGCACAACATCAAGCCTGT |
| | | GATTGTGTTTATTGATGATGTTGAACAAGTGGTAACTTTAAAGGATTCTGTATGCCAA |
| | | GTGAAAAAAAAAGATGTCTGACCTCCTTACATAT |
| 3 | Cyno CCR8 | MDYTLDPSMTTMTDYYYPDSLSSPCDGELIQRNDKLLLAVFYCLLFVFSLLGNSIVIL |
| | NP_001274549.1 | VLVVCKKLRNITDIYLLNLALSDLLEVESFPFQTYYQLDQWVFGTVMCKVVSGFYYIG |
| | | FYSSMFFITLMSVDRYLAVVHAVYAIKVRTIRMGTTLSLVVWLTAIMATIPLLVFYQV |
| | | ASEDGVLQCYSFYNQQTLKWKIFTNFEMNILGLLIPFTIFMFCYIKILHQLKRCQNHN |
| | | KTKAIRLVLIVVIASLLFWVPFNVVLFLTSLHSMHILDGCSISQQLNYATHVTEIISF |
| | | THCCVNPVIYAFVGEKFKKHLSEIFQKSCSHIFIYLGRQMPRESCEKSSSCQQHSFRS |
| | | SSIDYIL |
| 4 | Cyno CCR8 | ATGGATTATACACTTGACCCCAGCATGACAACAATGACCGACTACTACTACCCTGATA |
| | NM_001287620.1 | GCCTCTCAAGCCCCTGTGATGGAGAACTTATCCAGAGAAACGACAAGTTGCTCCTTGC |
| | | TGTCTTTTATTGCCTCCTGTTTGTATTCAGTCTTCTGGGAAACAGCCTGGTCATCCTG |
| | | GTCCTTGTGGTCTGCAAGAAGCTGAGGAACATCACAGACATATACCTCTTGAACCTGG |
| | | CCCTGTCTGACCTGCTTTTTGTCTTCTCCTTCCCCTTTCAGACCTACT |
| | | ATCAGCTGGATCAGTGGGTGTTTGGGACTGTAATGTGCAAAGTGGTGTCTGGCTTTTA |
| | | TTACATTGGCTTCTACAGCAGCATGTTTTTCATCACCCTCATGAGTGTGGACAGGTAC |
| | | CTGGCTGTTGTCCATGCCGTGTATGCCATAAAAGTGAGGACGATCAGGATGGGCACAA |
| | | CCCTGAGCCTGGTAGTATGGCTAACCGCCATTATGGCTACCATCCCATTGCTAGTGTT |
| | | TTACCAAGTGGCCTCTGAAGATGGTGTTCTACAGTGTTATTCATTTTA |
| | | CAATCAACAGACTTTGAAGTGGAAGATCTTCACCAACTTTGAAATGAACATTTTAGGC |
| | | TTGTTGATCCCATTCACCATCTTTATGTTCTGCTACATTAAAATCCTGCACCAGCTGA |
| | | AGAGGTGTCAAAACCACAACAAGACCAAGGCCATCAGGTTGGTGCTCATTGTGGTCAT |
| | | TGCATCTTTACTTTTCTGGGTCCCATTCAACGTGGTTCTTTTCCTCACTTCCTTGCAC |
| | | AGTATGCACATCTTGGATGGATGTAGCATAAGTCAACAACTGAATTAT |
| | | GCCACCCATGTCACAGAAATCATTTCCTTTACTCACTGCTGTGTGAACCCTGTTATCT |
| | | ATGCTTTTGTAGGGGAGAAGTTCAAGAAACACCTCTCAGAAATATTTCAGAAAAGTTG |
| | | CAGCCATATCTTCATCTACCTAGGAAGACAAATGCCTAGGGAGAGCTGTGAAAAGTCA |
| | | TCATCCTGCCAGCAGCACTCCTTCCGTTCCTCCAGCATAGACTACATTTTGTGA |
| 5 | Mouse | MDYTMEPNVTMTDYYPDFFTAPCDAEFLLRGSMLYLAILYCVLFVLGLLGNSLVILVL |
| | CCR8 | VGCKKLRSITDIYLLNLAASDLLFVLSIPFQTHNLLDQWVFGTAMCKVVSGLYYIGFF |
| | NP_031746.1 | SSMFFITLMSVDRYLAIVHAVYAIKVRTASVGTALSLTVWLAAVTATIPLMVFYQVAS |
| | | EDGMLQCFQFYEEQSLRWKLFTHFEINALGLLLPFAILLFCYVRILQQLRGCLNHNRT |
| | | RAIKLVLTVVIVSLLFWVPFNVALFLTSLHDLHILDGCATRQRLALAIHVTEVISFTH |
| | | CCVNPVIYAFIGEKFKKHLMDVFQKSCSHIFLYLGRQMPVGALERQLSSNQRSSHSST |
| | | LDDIL |
| 6 | Mouse | TGGCAGAGGAGTGGGCAGCTCTGAAACCTCAGAAGAAAGGCTCGCTCAGATAATTGGT |
| | CCR8 | CTTCCTGCCTCGATGGATTACACGATGGAGCCCAACGTCACGATGACCGACTACTACC |
| | NM_007720.2 | CTGATTTCTTCACCGCCCCCTGTGACGCAGAGTTCCTCCTCAGGGGCAGCATGCTGTA |
| | | TCTGGCCATCTTGTACTGCGTCTTGTTTGTGCTGGGCCTTCTGGGGAACAGCCTGGTC |
| | | ATCTTAGTCCTCGTGGGCTGCAAGAAACTGAGGAGCATCACAGATATC |
| | | TACCTCCTGAACCTGGCCGCATCCGACCTGCTCTTTGTCCTCTCTATTCCTTTTCAGA |
| | | CCCACAACCTGCTGGACCAGTGGGTGTTTGGGACTGCGATGTGTAAGGTGGTCTCTGG |
| | | CCTTTATTACATTGGTTTTTTCAGCAGTATGTTCTTCATCACCCTAATGAGTGTGGAC |
| | | AGGTATCTGGCTATTGTCCACGCTGTCTATGCCATCAAGGTGAGGACGGCCAGCGTGG |
| | | GCACAGCCCTGAGTCTGACAGTGTGGCTGGCTGCTGTCACAGCCACCA |
| | | TCCCCTTGATGGTTTTTTACCAAGTGGCCTCTGAAGACGGCATGCTACAATGTTTCCA |
| | | GTTTTATGAAGAGCAGTCTTTGAGGTGGAAGCTCTTTACCCACTTTGAAATCAACGCC |
| | | TTGGGTCTGCTGCTCCCCTTTGCCATCCTCCTGTTCTGCTATGTCAGGATCCTGCAGC |
| | | AGCTGCGGGGCTGCCTGAACCACAACAGGACCAGAGCCATCAAGCTGGTGCTCACCGT |
| | | AGTCATTGTGTCTTTACTCTTCTGGGTCCCATTCAACGTGGCCCTTTT |
| | | CCTCACGTCCCTGCACGACCTGCACATCTTGGATGGATGTGCCACGAGGCAGAGGCTG |
| | | GCTCTGGCCATCCATGTCACAGAGGTCATCTCTTTTACCCACTGCTGCGTGAACCCCG |
| | | TCATCTACGCGTTCATAGGAGAGAAGTTTAAGAAACACCTCATGGATGTGTTTCAAAA |
| | | GAGCTGCAGCCACATCTTCCTCTACTTAGGGAGACAAATGCCCGTGGGGGCGTTGGAA |
| | | AGGCAGCTGTCCTCGAACCAGCGATCTTCCCATTCTTCCACCCTGGAT |
| | | GACATCTTGTAAGGGGAGTGTGCAGGGCAGGCAGAC |
| 7 | Rat CCR8 | MDYTLEPNVTMTDYYPDFFTTPCDTELLLRGGTLYLAVLYCILFVLGLLGNSLVILVL |
| | XP_008764924.1 | VACKKLRSITDVYLLNLAASDLLFVLSIPFQTHNLLDQWVFGTVMCKVVSGLYYIGFF |
| | | SSMLFITLMSVDRYLAVVHPVHAIKVRTARVGTALSLAVWLAAIAATVPLMVFYQVSS |
| | | EDGMLQCFQLYDEQSLRWKLFTHFEVNALGLLLPFAILLFCYVRILQQLRGCLNHNRT |
| | | RAIKLVLTIVVVSLLFWVPFNVVLFLTSLHDMHILEGCATRQRLALATHVTEVISFMH |
| | | CCVNPVIYAFIGEKFKKHLVDVFQKSCSHIFLYVGRQMPVGALERQLSSNQRSSHSST |
| | | LDYIL |
| 8 | Rat CCR8 | CAGACATGCGGCAGAGGAGTGGGCAGCTCTGAAACCTCAGAAGGAAGGCTCGCTCACC |
| | XM_008766702.2 | TACCTGGTTTTCCCGCCTCGATGGATTACACGTTGGAGCCCAATGTCACGATGACTGA |
| | | CTACTACCCTGACTTCTTCACCACCCCCTGTGACACAGAGCTCCTCCTCAGGGGTGGC |
| | | ACGTTGTATCTGGCCGTCTTATACTGCATCTTGTTTGTGCTGGGCCTTCTGGGAAACA |
| | | GCCTGGTCATCTTGGTCCTTGTGGCCTGCAAGAAACTGAGGAGTATCA |
| | | CGGACGTCTACCTCCTGAACCTGGCCGCTTCTGACCTGCTCTTCGTCCTCTCCATTCC |
| | | CTTTCAGACCCACAACCTGCTGGACCAGTGGGTGTTTGGGACCGTGATGTGTAAGGTG |
| | | GTCTCTGGCCTCTACTACATTGGCTTCTTCAGCAGCATGCTCTTCATCACCCTCATGA |
| | | GTGTGGACAGGTACCTGGCTGTCGTCCACCCTGTCCATGCCATCAAAGTGAGGACGGC |
| | | CAGAGTGGGCACAGCCCTGAGCCTGGCAGTGTGGCTGGCTGCCATCGC |
| | | GGCCACCGTCCCACTGATGGTTTTTTACCAGGTGTCCTCTGAAGACGGCATGCTACAG |
| | | TGCTTCCAACTTTACGACGAGCAGTCTCTGAGGTGGAAGCTCTTCACCCACTTTGAAG |
| | | TCAACGCCTTGGGTCTGCTGCTCCCCTTTGCCATCCTCCTGTTCTGCTACGTCAGGAT |
| | | CCTGCAGCAGCTGCGGGGTTGCCTGAACCACAACAGGACCAGAGCCATCAAACTGGTG |
| | | CTCACCATAGTCGTCGTGTCTTTACTCTTCTGGGTCCCATTCAACGTG |
| | | GTCCTCTTCCTCACGTCCCTGCACGACATGCACATCTTGGAGGGATGTGCCACCAGGC |
| | | AGAGGCTGGCCCTGGCCACCCACGTCACGGAGGTCATCTCTTTCATGCATTGCTGCGT |
| | | GAACCCTGTCATCTATGCTTTCATCGGAGAGAAGTTCAAGAAGCACCTCGTGGATGTG |
| | | TTTCAAAAGAGCTGCAGCCACATCTTCCTCTACGTCGGGAGACAGATGCCAGTGGGGG |
| | | CGTTGGAAAGGCAACTGTCCTCGAACCAGCGATCTTCCCACTCTTCCACACTGGACTA |
| | | CATCTTGTAAGGGGGGTGGTGTGCACGACAGGCAGCCTCCACCTACATTGCCCTTCCT |
| | | GCTCCCAATCTTCTCCCCCCACCTCCC |

| | | | SEQ |
|---|---|---|---|
| | | | ID |
| Antibody | Region | Sequence | NO: |
| I2676 | VH CDR1 | GFTFSSYAMH | 9 |
| | VH CDR2 | AVISYDGSNKYYADSVKG | 10 |
| | VH CDR3 | ARVRDRAFDI | 11 |
| | VL CDR1 | TLRSGINVGTYRIY | 12 |
| | VL CDR2 | YKSDSDKQQGS | 13 |
| | VL CDR3 | WHSSARNWV | 14 |
| I2677 | VH CDR1 | SYGMH | 15 |
| | VH CDR2 | VISYDGSNKYYADSVKG | 16 |
| | VH CDR3 | DRRGGGYGDY | 17 |
| | VL CDR1 | TLRSGINVGTYRIY | 12 |
| | VL CDR2 | YKSDSDKQQGS | 13 |
| | VL CDR3 | MIWHSSARNWV | 20 |
| I3144 | VH CDR1 | SYAMH | 21 |
| | VH CDR2 | VISYDGSNKYYADSVKG | 16 |
| | VH CDR3 | VRDRAFDI | 23 |
| | VL CDR1 | TLRSGINVGTYRIY | 12 |
| | VL CDR2 | YKSDSDKQQGS | 13 |
| | VL CDR3 | MIWHSSARNWV | 20 |
| I3145 | VH CDR1 | SNYMS | 27 |
| | VH CDR2 | VIYSGGSTYYADSVKG | 28 |
| | VH CDR3 | GLGSADY | 29 |
| | VL CDR1 | RSSQSLLHSNGNTYLN | 30 |
| | VL CDR2 | KVSIRDS | 31 |
| | VL CDR3 | MQSTQWPIT | 32 |
| I3210 | VH CDR1 | GFTFSSYAMH | 9 |
| | VH CDR2 | AVISYDGSNKYYADSVKG | 10 |
| | VH CDR3 | ARVRDRAFDI | 11 |
| | VL CDR1 | TLRSGINVGTYRIY | 12 |
| | VL CDR2 | IIKSGSSDKQQGS | 37 |
| | VL CDR3 | WHSSARNWV | 14 |
| I3213 | VH CDR1 | GFTFSSYAMH | 9 |
| | VH CDR2 | AVISYDGSNKYYADSVKG | 10 |
| | VH CDR3 | ARVRDRAFDI | 11 |
| | VL CDR1 | TLRSGINLGTYRIY | 42 |
| | VL CDR2 | YKSDSDKQQGS | 13 |
| | VL CDR3 | WHSSARNWV | 14 |
| I2676 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |
| I2677 | VH FR1 | EVQLVESGGGVVQPGRSLRLSCAASGFTFS | 35 |
| | VH FR2 | WVRQAPGKGLEWVA | 36 |
| | VH FR3 | RFTISRDNSKNTLYLQMNSLRAEDTAVYYCAK | 38 |
| | VH FR4 | WGQGTLVTVSS | 39 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTQLTVL | 40 |
| I3144 | VH FR1 | QVQLVESGGGVVQPGRSLRLSCAASGFTFS | 41 |
| | VH FR2 | WVRQAPGKGLEWVA | 36 |
| | VH FR3 | RFTISRDNSKNTLYLQMNSLRAEDTAVYYCAR | 43 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVL | 44 |
| I3145 | VH FR1 | EVQLVETGGGLIQPGGSLRLSCAASGFTVS | 47 |
| | VH FR2 | WVRQAPGKGLEWVS | 53 |
| | VH FR3 | RFTISRDNSKNTLYLQMNSLRAEDTAVYYCAR | 43 |
| | VH FR4 | WGQGTLVTVSS | 39 |
| | VL FR1 | DVVMTQSPLSLPVTLGQPASISC | 49 |
| | VL FR2 | WFQQRPGQSPRRLIY | 50 |
| | VL FR3 | GVPDRFSGSGSGTDFTLKISRVEAEDVGLYYC | 69 |
| | VL FR4 | FGGGTKLEIK | 70 |
| I3210 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |
| I3213 | VH FR1 | QVQLVESGGGVVQPGRSLRLSCAAS | 18 |
| | VH FR2 | WVRQAPGKGLEWV | 19 |
| | VH FR3 | RFTISRDNSKNTLYLQMNSLRAEDTAVYYC | 22 |
| | VH FR4 | WGQGTMVTVSS | 24 |
| | VL FR1 | QAVLTQPASLSASPGASASLTC | 25 |
| | VL FR2 | WYQQKPGSPPQYLLR | 26 |
| | VL FR3 | GVPSRFSGSKDASANAGILLISGLQSEDEADYYC | 33 |
| | VL FR4 | FGGGTKLTVLG | 34 |
| I2676 | VH | QVQLVESGGGVVQPGRSLRLSCAASGFTFSSYAMHWV | 45 |
| | | RQAPGKGLEWVAVISYDGSNKYYADSVKGRFTISRDNS | |
| | | KNTLYLQMNSLRAEDTAVYYCARVRDRAFDIWGQGT | |
| | | MVTVSS | |
| | VL | QAVLTQPASLSASPGASASLTCTLRSGINVGTYRIYWYQ | 51 |
| | | QKPGSPPQYLLRYKSDSDKQQGSGVPSRFSGSKDASAN | |
| | | AGILLISGLQSEDEADYYCMIWHSSARNWVFGGGTKLT | |
| | | VL | |
| I2677 | VH | EVQLVESGGGVVQPGRSLRLSCAASGFTFSSYGMHWVR | 46 |
| | | QAPGKGLEWVAVISYDGSNKYYADSVKGRFTISRDNSK | |
| | | NTLYLQMNSLRAEDTAVYYCAKDRRGGGYGDYWGQG | |
| | | TLVTVSS | |
| | VL | QAVLTQPASLSASPGASASLTCTLRSGINVGTYRIYWYQ | 52 |
| | | QKPGSPPQYLLRYKSDSDKQQGSGVPSRFSGSKDASAN | |
| | | AGILLISGLQSEDEADYYCMIWHSSARNWVFGGGTQLT | |
| | | VL | |
| I3144 | VH | QVQLVESGGGVVQPGRSLRLSCAASGFTFSSYAMHWV | 45 |
| | | RQAPGKGLEWVAVISYDGSNKYYADSVKGRFTISRDNS | |
| | | KNTLYLQMNSLRAEDTAVYYCARVRDRAFDIWGQGT | |
| | | MVTVSS | |
| | VL | QAVLTQPASLSASPGASASLTCTLRSGINVGTYRIYWYQ | 51 |
| | | QKPGSPPQYLLRYKSDSDKQQGSGVPSRFSGSKDASAN | |
| | | AGILLISGLQSEDEADYYCMIWHSSARNWVFGGGTKLT | |
| | | VL | |
| I3145 | VH | EVQLVETGGGLIQPGGSLRLSCAASGFTVSSNYMSWVR | 48 |
| | | QAPGKGLEWVSVIYSGGSTYYADSVKGRFTISRDNSKN | |
| | | TLYLQMNSLRAEDTAVYYCARGLGSADYWGQGTLVT | |
| | | VSS | |
| | VL | DVVMTQSPLSLPVTLGQPASISCRSSQSLLHSNGNTYLN | 54 |
| | | WFQQRPGQSPRRLIYKVSIRDSGVPDRFSGSGSGTDFTL | |
| | | KISRVEAEDVGLYYCMQSTQWPITFGGGTKLEIK | |
| I3210 | VH | QVQLVESGGGVVQPGRSLRLSCAASGFTFSSYAMHWV | 45 |
| | | RQAPGKGLEWVAVISYDGSNKYYADSVKGRFTISRDNS | |
| | | KNTLYLQMNSLRAEDTAVYYCARVRDRAFDIWGQGT | |
| | | MVTVSS | |
| | VL | QAVLTQPASLSASPGASASLTCTLRSGINVGTYRIYWYQ | 55 |
| | | QKPGSPPQYLLRIIKSGSSDKQQGSGVPSRFSGSKDASAN | |
| | | AGILLISGLQSEDEADYYCMIWHSSARNWVFGGGTKLT | |
| | | VLG | |
| I3213 | VH | QVQLVESGGGVVQPGRSLRLSCAASGFTFSSYAMHWV | 45 |
| | | RQAPGKGLEWVAVISYDGSNKYYADSVKGRFTISRDNS | |
| | | KNTLYLQMNSLRAEDTAVYYCARVRDRAFDIWGQGT | |
| | | MVTVSS | |
| | VL | QAVLTQPASLSASPGASASLTCTLRSGINLGTYRIYWYQ | 56 |
| | | QKPGSPPQYLLRYKSDSDKQQGSGVPSRFSGSKDASAN | |
| | | AGILLISGLQSEDEADYYCMIWHSSARNWVFGGGTKLT | |
| | | VLG | |
| I2676 | VH | CAAGTGCAGCTGGTCGAGAGCGGAGGAGGAGTGGTG | 57 |
| | | CAGCCCGGCAGATCTCTGAGACTGAGCTGTGCCGCCT | |
| | | CCGGCTTCACCTTCAGCAGCTACGCCATGCACTGGGT | |
| | | GAGACAAGCCCCCGGCAAGGGACTGGAATGGGTGGC | |
| | | CGTCATCTCCTACGACGGCTCCAACAAGTACTACGCC | |
| | | GACAGCGTGAAGGGAAGATTCACCATCTCTAGAGAC | |
| | | AACAGCAAGAACACACTGTATCTGCAGATGAACTCTC | |
| | | TGAGAGCTGAGGACACAGCCGTGTACTATTGCGCTAG | |
| | | GGTGAGAGATAGAGCCTTCGACATCTGGGGCCAAGG | |
| | | CACCATGGTGACCGTGAGCTCA | |
| | | (SEQ ID NO: 57) | |
| | VL | CAAGCCGTGCTGACACAACCCGCCAGCCTCAGCGCCA | 58 |
| | | GCCCCGGCGCTAGCGCTTCTCTGACATGCACACTGAG | |
| | | GTCCGGCATCAACGTGGGCACCTATAGAATCTACTGG | |
| | | TACCAGCAGAAACCCGGCTCCCCTCCTCAGTATCTGC | |
| | | TGAGGTACAAGTCCGATAGCGACAAGCAGCAAGGCT | |
| | | CCGGCGTGCCTTCTAGATTTAGCGGCAGCAAGGATGC | |
| | | CAGCGCCAATGCCGGCATTCTGCTGATCAGCGGACTG | |
| | | CAGAGCGAGGATGAGGCCGACTACTACTGCATGATCT | |
| | | GGCACTCCAGCGCCAGAAACTGGGTGTTCGGCGGCG | |
| | | GAACCAAGCTGACCGTGCTA | |
| I2677 | VH | GAGGTGCAGCTGGTGGAAAGCGGAGGCGGAGTGGTG | 59 |
| | | CAGCCCGGCAGATCTCTGAGGCTGAGCTGTGCCGCTA | |
| | | GCGGCTTCACCTTCAGCAGCTACGGCATGCACTGGGT | |
| | | GAGGCAAGCCCCCGGCAAGGGACTGGAGTGGGTCGC | |
| | | CGTGATCAGCTACGACGGCAGCAACAAGTACTACGC | |
| | | CGACAGCGTGAAGGGAAGATTCACCATCTCTAGAGA | |
| | | CAACAGCAAGAACACCCTCTACCTCCAGATGAACTCT | |
| | | CTGAGGGCCGAGGATACCGCCGTGTACTACTGCGCCA | |
| | | AGGACAGAAGAGGCGGCGGATACGGCGATTACTGGG | |
| | | GCCAAGGCACACTGGTGACAGTGAGCTCA | |
| | VL | CAAGCCGTGCTGACCCAGCCCGCCTCTCTGAGCGCTA | 60 |
| | | GCCCCGGCGCCTCCGCTTCTCTGACATGCACACTGAG | |
| | | GTCCGGAATCAACGTGGGCACCTATAGAATCTACTGG | |
| | | TACCAGCAGAAGCCCGGCAGCCCTCCTCAGTATCTGC | |
| | | TGAGATACAAGAGCGACAGCGATAAGCAGCAAGGCT | |
| | | CCGGAGTGCCTAGCAGATTCAGCGGCAGCAAAGACG | |
| | | CCAGCGCCAATGCCGGAATTCTGCTGATCAGCGGACT | |
| | | GCAGAGCGAGGACGAAGCCGACTACTACTGCATGAT | |
| | | CTGGCACTCCAGCGCCAGAAACTGGGTGTTTGGCGGC | |
| | | GGCACCCAGCTGACAGTGCTA | |
| I3144 | VH | CAAGTGCAGCTGGTCGAGAGCGGAGGAGGAGTGGTG | 61 |
| | | CAGCCCGGCAGATCTCTGAGGCTGAGCTGCGCCGCCA | |
| | | GCGGATTCACCTTCAGCTCCTACGCCATGCACTGGGT | |
| | | GAGACAAGCCCCCGGCAAGGGACTGGAGTGGGTGGC | |
| | | CGTGATTTCCTACGACGGCTCCAACAAGTACTACGCC | |
| | | GACAGCGTGAAGGGAAGATTCACCATCTCTAGAGAC | |
| | | AACAGCAAGAACACACTGTATCTGCAGATGAACTCTC | |
| | | TGAGAGCCGAGGACACCGCCGTGTACTACTGCGCCA | |
| | | GAGTGAGGGACAGAGCCTTCGACATTTGGGGCCAAG | |
| | | GCACCATGGTGACAGTGAGCTCA | |
| | VL | CAAGCCGTGCTGACCCAGCCCGCCTCTCTGAGCGCTA | 62 |
| | | GCCCCGGCGCTAGCGCTTCTCTGACATGCACACTGAG | |
| | | GAGCGGCATCAACGTGGGCACCTATAGAATCTACTGG | |
| | | TACCAGCAGAAGCCCGGCAGCCCTCCTCAGTATCTGC | |
| | | TGAGATACAAGTCCGACAGCGACAAGCAGCAAGGCA | |
| | | GCGGCGTGCCTTCTAGATTCAGCGGCAGCAAGGACGC | |
| | | CAGCGCTAATGCCGGCATTCTGCTGATCAGCGGACTG | |
| | | CAGAGCGAGGATGAGGCCGACTACTACTGCATGATCT | |
| | | GGCACAGCAGCGCCAGAAACTGGGTGTTCGGCGGCG | |
| | | GCACCAAGCTGACAGTGCTA | |
| I3145 | VH | GAGGTGCAGCTGGTGGAAACCGGCGGCGGACTGATT | 63 |
| | | CAGCCCGGAGGATCTCTGAGGCTGAGCTGTGCCGCTA | |
| | | GCGGCTTCACCGTGAGCAGCAACTATATGAGCTGGGT | |
| | | GAGACAAGCCCCCGGCAAAGGACTGGAGTGGGTGAG | |
| | | CGTGATCTACAGCGGCGGCAGCACATACTACGCCGAC | |
| | | AGCGTGAAGGGAAGATTCACCATCTCTAGAGACAAC | |
| | | AGCAAGAACACACTGTATCTGCAGATGAACTCTCTGA | |
| | | GGGCCGAGGACACCGCCGTGTACTACTGCGCCAGAG | |
| | | GACTGGGCAGCGCTGATTACTGGGGCCAAGGCACAC | |
| | | TGGTGACAGTGTCCTCA | |
| | VL | GACGTGGTGATGACCCAGAGCCCTCTGTCTCTGCCCG | 64 |
| | | TGACACTGGGACAGCCCGCCAGCATCAGCTGCAGAA | |
| | | GCTCCCAGTCTCTGCTGCACAGCAATGGCAACACCTA | |
| | | TCTGAACTGGTTCCAGCAAAGACCCGGCCAGTCCCCC | |
| | | AGAAGGCTGATCTACAAGGTGAGCATTAGAGATAGC | |
| | | GGCGTGCCCGACAGATTTAGCGGCAGCGGAAGCGGC | |
| | | ACAGACTTCACACTGAAGATCTCTAGAGTGGAGGCTG | |
| | | AGGACGTGGGACTGTACTACTGCATGCAGAGCACCC | |
| | | AGTGGCCCATCACCTTTGGCGGCGGCACCAAGCTGGA | |
| | | GATCAAA | |
| I3210 | VH | CAAGTGCAGCTGGTCGAGAGCGGAGGAGGAGTGGTG | 65 |
| | | CAGCCCGGCAGATCTCTGAGGCTGAGCTGCGCCGCCA | |
| | | GCGGATTCACCTTCAGCTCCTACGCCATGCACTGGGT | |
| | | GAGACAAGCCCCCGGCAAGGGACTGGAGTGGGTGGC | |
| | | CGTGATTTCCTACGACGGCTCCAACAAGTACTACGCC | |
| | | GACAGCGTGAAGGGAAGATTCACCATCTCTAGGGAC | |
| | | AACAGCAAGAACACACTGTATCTGCAGATGAACTCTC | |
| | | TGAGAGCCGAGGACACCGCCGTGTACTACTGCGCCA | |
| | | GAGTGAGGGACAGAGCCTTCGACATTTGGGGCCAAG | |
| | | GCACCATGGTGACAGTGAGCTCA | |
| | VL | CAAGCCGTGCTGACCCAGCCCGCCTCTCTGAGCGCTA | 66 |
| | | GCCCCGGCGCTAGCGCCTCTCTGACATGCACACTGAG | |
| | | AAGCGGCATCAACGTGGGCACCTATAGAATCTACTGG | |
| | | TACCAGCAGAAACCCGGCTCCCCC | |
| | | CCTCAGTATCTGCTGAGAATCATCAAGAGCGGCAGCA | |
| | | GCGACAAACAGCAAGGCAGCGGCGTGCCTAGCAGAT | |
| | | TCAGCGGCTCCAAGGATGCCAGCGCCAATGCCGGCAT | |
| | | TCTGCTGATCTCCGGACTGCAGAGCGAGGACGAGGCC | |
| | | GACTACTACTGCATGATCTGGCACAGCTCCGCCAGAA | |
| | | ACTGGGTGTTCGGCGGCGGCACAAAGCTGACAGTGCT | |
| | | GGGC | |
| I3213 | VH | CAAGTGCAGCTGGTCGAGAGCGGAGGAGGAGTGGTG | 67 |
| | | CAGCCCGGCAGATCTCTGAGGCTGAGCTGCGCCGCCA | |
| | | GCGGATTCACCTTCAGCTCCTACGCCATGCACTGGGT | |
| | | GAGACAAGCCCCCGGCAAGGGACTGGAGTGGGTGGC | |
| | | CGTGATTTCCTACGACGGCTCCAACAAGTACTACGCC | |
| | | GACAGCGTGAAGGGAAGATTCACCATCTCTAGGGAC | |
| | | AACAGCAAGAACACACTGTATCTGCAGATGAACTCTC | |
| | | TGAGAGCCGAGGACACCGCCGTGTACTACTGCGCCA | |
| | | GAGTGAGGGACAGAGCCTTCGACATTTGGGGCCAAG | |
| | | GCACCATGGTGACAGTGAGCTCA | |
| | VL | CAAGCCGTGCTGACCCAGCCCGCCTCTCTGAGCGCTA | 68 |
| | | GCCCCGGCGCTTCCGCCTCTCTGACATGCACACTGAG | |
| | | GTCCGGCATCAATCTGGGCACCTATAGAATCTACTGG | |
| | | TACCAGCAGAAGCCCGGCAGCCCTCCCCAGTATCTGC | |
| | | TGAGGTACAAGAGCGACAGCGATAAGCAGCAAGGCA | |
| | | GCGGCGTGCCTAGCAGATTTAGCGGAAGCAAGGACG | |
| | | CCTCCGCTAATGCCGGCATTCTGCTGATCAGCGGACT | |
| | | GCAGAGCGAGGATGAGGCCGACTACTACTGCATGAT | |
| | | CTGGCACTCCTCCGCCAGAAACTGGGTGTTCGGCGGA | |
| | | GGCACCAAGCTGACAGTGCTGGGC | |
| I2676 | Heavy chain | MAVLGLLLCLVTFPSCVLS | 71 |
| | (leader | QVQLVESGGGVVQPGRSLRLSCAASGFTFSSYAMHWV | |
| | sequence- | RQAPGKGLEWVAVISYDGSNKYYADSVKGRFTISRDNS | |
| | HCVR- | KNTLYLQMNSLRAEDTAVYYCARVRDRAFDIWGQGT | |
| | constant | MVTVSS | |
| | region) | ASTKGPSVFPLAPSSKSTSGGTAALGCLVKDYFPEPVTV | |
| | | SWNSGALTSGVHTFPAVLQSSGLYSLSSVVTVPSSSLGT | |
| | | QTYICNVNHKPSNTKVDKKVEPKSCDKTHTCPPCPAPEL | |
| | | LGGPSVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPIEKTISKAKGQPREPQV | |
| | | YTLPPSRDELTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| I2676 | Heavy chain | MAVLGLLLCLVTFPSCVLS | 72 |
| | with S239D | QVQLVESGGGVVQPGRSLRLSCAASGFTFSSYAMHWV | |
| | I332E Fc | RQAPGKGLEWVAVISYDGSNKYYADSVKGRFTISRDNS | |
| | mutations | KNTLYLQMNSLRAEDTAVYYCARVRDRAFDIWGQGT | |
| | (leader | MVTVSS | |
| | sequence- | ASTKGPSVFPLAPSSKSTSGGTAALGCLVKDYFPEPVTV | |
| | HCVR- | SWNSGALTSGVHTFPAVLQSSGLYSLSSVVTVPSSSLGT | |
| | constant | QTYICNVNHKPSNTKVDKKVEPKSCDKTHTCPPCPAPEL | |
| | region) | LGGPDVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPEEKTISKAKGQPREPQV | |
| | | YTLPPSRDELTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| I2676 | Light chain | METDTLLLWVLLLWVPGSTG | 73 |
| | (leader | QAVLTQPASLSASPGASASLTCTLRSGINVGTYRIYWYQ | |
| | sequence- | QKPGSPPQYLLRYKSDSDKQQGSGVPSRESGSKDASAN | |
| | LCVR- | AGILLISGLQSEDEADYYCMIWHSSARNWVFGGGTKLT | |
| | constant | VL | |
| | region) | GQPKAAPSVTLFPPSSEELQANKATLVCLISDFYPGAVT | |
| | | VAWKADSSPVKAGVETTTPSKQSNNKYAASSYLSLTPE | |
| | | QWKSHRSYSCQVTHEGSTVEKTVAPTECS | |
| I2676 | Heavy chain | QVQLVESGGGVVQPGRSLRLSCAASGFTESSYAMHWV | 74 |
| | (HCVR- | RQAPGKGLEWVAVISYDGSNKYYADSVKGRFTISRDNS | |
| | constant | KNTLYLQMNSLRAEDTAVYYCARVRDRAFDIWGQGT | |
| | region) | MVTVSS | |
| | | ASTKGPSVFPLAPSSKSTSGGTAALGCLVKDYFPEPVTV | |
| | | SWNSGALTSGVHTFPAVLQSSGLYSLSSVVTVPSSSLGT | |
| | | QTYICNVNHKPSNTKVDKKVEPKSCDKTHTCPPCPAPEL | |
| | | LGGPSVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPIEKTISKAKGQPREPQV | |
| | | YTLPPSRDELTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| I2676 | Heavy chain | QVQLVESGGGVVQPGRSLRLSCAASGFTFSSYAMHWV | 75 |
| | with S239D | RQAPGKGLEWVAVISYDGSNKYYADSVKGRFTISRDNS | |
| | I332E Fc | KNTLYLQMNSLRAEDTAVYYCARVRDRAFDIWGQGT | |
| | mutations | MVTVSS | |
| | (HCVR- | ASTKGPSVFPLAPSSKSTSGGTAALGCLVKDYFPEPVTV | |
| | constant | SWNSGALTSGVHTFPAVLQSSGLYSLSSVVTVPSSSLGT | |
| | region) | QTYICNVNHKPSNTKVDKKVEPKSCDKTHTCPPCPAPEL | |
| | | LGGPDVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPEEKTISKAKGQPREPQV | |
| | | YTLPPSRDELTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| I2676 | Light chain | QAVLTQPASLSASPGASASLTCTLRSGINVGTYRIYWYQ | 76 |
| | (LCVR- | QKPGSPPQYLLRYKSDSDKQQGSGVPSRESGSKDASAN | |
| | constant | AGILLISGLQSEDEADYYCMIWHSSARNWVFGGGTKLT | |
| | region) | VL | |
| | | GQPKAAPSVTLFPPSSEELQANKATLVCLISDFYPGAVT | |
| | | VAWKADSSPVKAGVETTTPSKQSNNKYAASSYLSLTPE | |
| | | QWKSHRSYSCQVTHEGSTVEKTVAPTECS | |
| I2676 | Heavy chain | ATGGCTGTCCTGGGGCTGCTTCTCTGCCTGGTGAC | 77 |
| | (leader | GTTCCCAAGCTGTGTCTTAAGC | |
| | sequence- | CAAGTGCAGCTGGTCGAGAGCGGAGGAGGAGTGGTG | |
| | HCVR- | CAGCCCGGCAGATCTCTGAGACTGAGCTGTGCCGCCT | |
| | constant | CCGGCTTCACCTTCAGCAGCTACGCCATGCACTGGGT | |
| | region) | GAGACAAGCCCCCGGCAAGGGACTGGAATGGGTGGC | |
| | | CGTCATCTCCTACGACGGCTCCAACAAGTACTACGCC | |
| | | GACAGCGTGAAGGGAAGATTCACCATCTCTAGAGAC | |
| | | AACAGCAAGAACACACTGTATCTGCAGATGAACTCTC | |
| | | TGAGAGCTGAGGACACAGCCGTGTACTATTGCGCTAG | |
| | | GGTGAGAGATAGAGCCTTCGACATCTGGGGCCAAGG | |
| | | CACCATGGTGACCGTGAGCTCA | |
| | | GCCTCCACCAAGGGCCCATCGGTGTTCCCCCTGGCAC | |
| | | CCTCCTCCAAGAGCACCTCTGGGGGCACAGCGGCCCT | |
| | | GGGCTGCCTGGTCAAGGACTACTTCCCCGAACCGGTG | |
| | | ACGGTGTCGTGGAACTCAGGCGCCCTGACCAGCGGC | |
| | | GTGCACACCTTCCCGGCTGTCCTACAGTCCTCAGGAC | |
| | | TCTACTCCCTCAGCAGCGTGGTGACCGTGCCCTCCAG | |
| | | CAGCTTGGGCACCCAGACCTACATCTGCAACGTGAAT | |
| | | CACAAGCCCAGCAACACCAAGGTGGACAAGAAAGTT | |
| | | GAGCCCAAATCTTGTGACAAAACTCACACATGCCCAC | |
| | | CGTGCCCAGCACCTGAACTCCTGGGGGGACCGTCAGT | |
| | | GTTCCTCTTCCCCCCAAAACCCAAGGACACCCTCATG | |
| | | ATCTCCCGGACCCCTGAGGTCACATGCGTGGTGGTGG | |
| | | ACGTGAGCCACGAGGACCCTGAGGTCAAGTTCAACT | |
| | | GGTACGTGGACGGCGTGGAGGTGCATAATGCCAAGA | |
| | | CAAAGCCGCGGGAGGAGCAGTACAACAGCACGTACC | |
| | | GGGTGGTCAGCGTCCTCACCGTCCTGCACCAGGACTG | |
| | | GCTGAATGGCAAGGAGTACAAGTGCAAGGTGTCCAA | |
| | | CAAAGCCCTCCCAGCCCCCATCGAGAAAACCATCTCC | |
| | | AAAGCCAAAGGGCAGCCCCGAGAACCACAGGTGTAC | |
| | | ACCCTGCCCCCATCCCGGGATGAGCTGACCAAGAACC | |
| | | AGGTCAGCCTGACCTGCCTGGTCAAAGGCTTCTATCC | |
| | | CAGCGACATCGCCGTGGAGTGGGAGAGCAATGGGCA | |
| | | GCCGGAGAACAACTACAAGACCACGCCTCCCGTGCT | |
| | | GGACTCCGACGGCTCCTTCTTCCTCTACAGCAAGCTC | |
| | | ACCGTGGACAAGAGCAGGTGGCAGCAGGGGAACGTG | |
| | | TTCTCATGCTCCGTGATGCATGAGGCTCTGCACAACC | |
| | | ACTACACGCAGAAGAGCCTCTCCCTGTCTCCGGGTTA | |
| | | A | |
| I2676 | Heavy chain | ATGGCTGTCCTGGGGCTGCTTCTCTGCCTGGTGAC | 78 |
| | with S239D | GTTCCCAAGCTGTGTCTTAAGC | |
| | I332E Fc | CAAGTGCAGCTGGTCGAGAGCGGAGGAGGAGTGGTG | |
| | mutations | CAGCCCGGCAGATCTCTGAGACTGAGCTGTGCCGCCT | |
| | (leader | CCGGCTTCACCTTCAGCAGCTACGCCATGCACTGGGT | |
| | sequence- | GAGACAAGCCCCCGGCAAGGGACTGGAATGGGTGGC | |
| | HCVR- | CGTCATCTCCTACGACGGCTCCAACAAGTACTACGCC | |
| | constant | GACAGCGTGAAGGGAAGATTCACCATCTCTAGAGAC | |
| | region) | AACAGCAAGAACACACTGTATCTGCAGATGAACTCTC | |
| | | TGAGAGCTGAGGACACAGCCGTGTACTATTGCGCTAG | |
| | | GGTGAGAGATAGAGCCTTCGACATCTGGGGCCAAGG | |
| | | CACCATGGTGACCGTGAGCTCA | |
| | | GCCTCCACCAAGGGCCCATCGGTGTTCCCCCTGGCAC | |
| | | CCTCCTCCAAGAGCACCTCTGGGGGCACAGCGGCCCT | |
| | | GGGCTGCCTGGTCAAGGACTACTTCCCCGAACCGGTG | |
| | | ACGGTGTCGTGGAACTCAGGCGCCCTGACCAGCGGC | |
| | | GTGCACACCTTCCCGGCTGTCCTACAGTCCTCAGGAC | |
| | | TCTACTCCCTCAGCAGCGTGGTGACCGTGCCCTCCAG | |
| | | CAGCTTGGGCACCCAGACCTACATCTGCAACGTGAAT | |
| | | CACAAGCCCAGCAACACCAAGGTGGACAAGAAAGTT | |
| | | GAGCCCAAATCTTGTGACAAAACTCACACATGCCCAC | |
| | | CGTGCCCAGCACCTGAACTCCTGGGGGGACCGGATGT | |
| | | GTTCCTCTTCCCCCCAAAACCCAAGGACACCCTCATG | |
| | | ATCTCCCGGACCCCTGAGGTCACATGCGTGGTGGTGG | |
| | | ACGTGAGCCACGAGGACCCTGAGGTCAAGTTCAACT | |
| | | GGTACGTGGACGGCGTGGAGGTGCATAATGCCAAGA | |
| | | CAAAGCCGCGGGAGGAGCAGTACAACAGCACGTACC | |
| | | GGGTGGTCAGCGTCCTCACCGTCCTGCACCAGGACTG | |
| | | GCTGAATGGCAAGGAGTACAAGTGCAAGGTGTCCAA | |
| | | CAAAGCCCTCCCAGCCCCCGAGGAGAAAACCATCTCC | |
| | | AAAGCCAAAGGGCAGCCCCGAGAACCACAGGTGTAC | |
| | | ACCCTGCCCCCATCCCGGGATGAGCTGACCAAGAACC | |
| | | AGGTCAGCCTGACCTGCCTGGTCAAAGGCTTCTATCC | |
| | | CAGCGACATCGCCGTGGAGTGGGAGAGCAATGGGCA | |
| | | GCCGGAGAACAACTACAAGACCACGCCTCCCGTGCT | |
| | | GGACTCCGACGGCTCCTTCTTCCTCTACAGCAAGCTC | |
| | | ACCGTGGACAAGAGCAGGTGGCAGCAGGGGAACGTG | |
| | | TTCTCATGCTCCGTGATGCATGAGGCTCTGCACAACC | |
| | | ACTACACGCAGAAGAGCCTCTCCCTGTCTCCGGGTTA | |
| | | A | |
| I2676 | Light chain | ATGGAGACAGACACACTCCTGCTATGGGTGCTGCT | 79 |
| | (leader | GCTCTGGGTACCAGGTTCCACAGGT | |
| | sequence- | CAAGCCGTGCTGACACAACCCGCCAGCCTCAGCGCCA | |
| | LCVR- | GCCCCGGCGCTAGCGCTTCTCTGACATGCACACTGAG | |
| | constant | GTCCGGCATCAACGTGGGCACCTATAGAATCTACTGG | |
| | region) | TACCAGCAGAAACCCGGCTCCCCTCCTCAGTATCTGC | |
| | | TGAGGTACAAGTCCGATAGCGACAAGCAGCAAGGCT | |
| | | CCGGCGTGCCTTCTAGATTTAGCGGCAGCAAGGATGC | |
| | | CAGCGCCAATGCCGGCATTCTGCTGATCAGCGGACTG | |
| | | CAGAGCGAGGATGAGGCCGACTACTACTGCATGATCT | |
| | | GGCACTCCAGCGCCAGAAACTGGGTGTTCGGCGGCG | |
| | | GAACCAAGCTGACCGTGCTA | |
| | | GGCCAGCCCAAGGCTGCCCCCTCGGTCACTCTGTTCC | |
| | | CGCCCTCCTCTGAGGAGCTTCAAGCCAACAAGGCCAC | |
| | | ACTGGTGTGTCTCATAAGTGACTTCTACCCGGGAGCC | |
| | | GTGACAGTGGCCTGGAAGGCAGATAGCAGCCCCGTC | |
| | | AAGGCGGGAGTGGAAACCACCACACCCTCCAAACAA | |
| | | AGCAACAACAAGTACGCGGCCAGCAGCTACCTGAGC | |
| | | CTGACGCCCGAGCAGTGGAAGTCCCACAGAAGCTAC | |
| | | AGCTGCCAGGTCACGCATGAAGGGAGCACCGTGGAA | |
| | | AAGACAGTGGCCCCTACAGAATGTTCATAA | |
| I2676 | Heavy chain | CAAGTGCAGCTGGTCGAGAGCGGAGGAGGAGTGGTG | 80 |
| | (HCVR- | CAGCCCGGCAGATCTCTGAGACTGAGCTGTGCCGCCT | |
| | constant | CCGGCTTCACCTTCAGCAGCTACGCCATGCACTGGGT | |
| | region) | GAGACAAGCCCCCGGCAAGGGACTGGAATGGGTGGC | |
| | | CGTCATCTCCTACGACGGCTCCAACAAGTACTACGCC | |
| | | GACAGCGTGAAGGGAAGATTCACCATCTCTAGAGAC | |
| | | AACAGCAAGAACACACTGTATCTGCAGATGAACTCTC | |
| | | TGAGAGCTGAGGACACAGCCGTGTACTATTGCGCTAG | |
| | | GGTGAGAGATAGAGCCTTCGACATCTGGGGCCAAGG | |
| | | CACCATGGTGACCGTGAGCTCA | |
| | | GCCTCCACCAAGGGCCCATCGGTGTTCCCCCTGGCAC | |
| | | CCTCCTCCAAGAGCACCTCTGGGGGCACAGCGGCCCT | |
| | | GGGCTGCCTGGTCAAGGACTACTTCCCCGAACCGGTG | |
| | | ACGGTGTCGTGGAACTCAGGCGCCCTGACCAGCGGC | |
| | | GTGCACACCTTCCCGGCTGTCCTACAGTCCTCAGGAC | |
| | | TCTACTCCCTCAGCAGCGTGGTGACCGTGCCCTCCAG | |
| | | CAGCTTGGGCACCCAGACCTACATCTGCAACGTGAAT | |
| | | CACAAGCCCAGCAACACCAAGGTGGACAAGAAAGTT | |
| | | GAGCCCAAATCTTGTGACAAAACTCACACATGCCCAC | |
| | | CGTGCCCAGCACCTGAACTCCTGGGGGGACCGTCAGT | |
| | | GTTCCTCTTCCCCCCAAAACCCAAGGACACCCTCATG | |
| | | ATCTCCCGGACCCCTGAGGTCACATGCGTGGTGGTGG | |
| | | ACGTGAGCCACGAGGACCCTGAGGTCAAGTTCAACT | |
| | | GGTACGTGGACGGCGTGGAGGTGCATAATGCCAAGA | |
| | | CAAAGCCGCGGGAGGAGCAGTACAACAGCACGTACC | |
| | | GGGTGGTCAGCGTCCTCACCGTCCTGCACCAGGACTG | |
| | | GCTGAATGGCAAGGAGTACAAGTGCAAGGTGTCCAA | |
| | | CAAAGCCCTCCCAGCCCCCATCGAGAAAACCATCTCC | |
| | | AAAGCCAAAGGGCAGCCCCGAGAACCACAGGTGTAC | |
| | | ACCCTGCCCCCATCCCGGGATGAGCTGACCAAGAACC | |
| | | AGGTCAGCCTGACCTGCCTGGTCAAAGGCTTCTATCC | |
| | | CAGCGACATCGCCGTGGAGTGGGAGAGCAATGGGCA | |
| | | GCCGGAGAACAACTACAAGACCACGCCTCCCGTGCT | |
| | | GGACTCCGACGGCTCCTTCTTCCTCTACAGCAAGCTC | |
| | | ACCGTGGACAAGAGCAGGTGGCAGCAGGGGAACGTG | |
| | | TTCTCATGCTCCGTGATGCATGAGGCTCTGCACAACC | |
| | | ACTACACGCAGAAGAGCCTCTCCCTGTCTCCGGGTTA | |
| | | A | |
| I2676 | Heavy chain | CAAGTGCAGCTGGTCGAGAGCGGAGGAGGAGTGGTG | 81 |
| | with S239D | CAGCCCGGCAGATCTCTGAGACTGAGCTGTGCCGCCT | |
| | I332E Fc | CCGGCTTCACCTTCAGCAGCTACGCCATGCACTGGGT | |
| | mutations | GAGACAAGCCCCCGGCAAGGGACTGGAATGGGTGGC | |
| | (HCVR- | CGTCATCTCCTACGACGGCTCCAACAAGTACTACGCC | |
| | constant | GACAGCGTGAAGGGAAGATTCACCATCTCTAGAGAC | |
| | region) | AACAGCAAGAACACACTGTATCTGCAGATGAACTCTC | |
| | | TGAGAGCTGAGGACACAGCCGTGTACTATTGCGCTAG | |
| | | GGTGAGAGATAGAGCCTTCGACATCTGGGGCCAAGG | |
| | | CACCATGGTGACCGTGAGCTCA | |
| | | GCCTCCACCAAGGGCCCATCGGTGTTCCCCCTGGCAC | |
| | | CCTCCTCCAAGAGCACCTCTGGGGGCACAGCGGCCCT | |
| | | GGGCTGCCTGGTCAAGGACTACTTCCCCGAACCGGTG | |
| | | ACGGTGTCGTGGAACTCAGGCGCCCTGACCAGCGGC | |
| | | GTGCACACCTTCCCGGCTGTCCTACAGTCCTCAGGAC | |
| | | TCTACTCCCTCAGCAGCGTGGTGACCGTGCCCTCCAG | |
| | | CAGCTTGGGCACCCAGACCTACATCTGCAACGTGAAT | |
| | | CACAAGCCCAGCAACACCAAGGTGGACAAGAAAGTT | |
| | | GAGCCCAAATCTTGTGACAAAACTCACACATGCCCAC | |
| | | CGTGCCCAGCACCTGAACTCCTGGGGGGACCGGATGT | |
| | | GTTCCTCTTCCCCCCAAAACCCAAGGACACCCTCATG | |
| | | ATCTCCCGGACCCCTGAGGTCACATGCGTGGTGGTGG | |
| | | ACGTGAGCCACGAGGACCCTGAGGTCAAGTTCAACT | |
| | | GGTACGTGGACGGCGTGGAGGTGCATAATGCCAAGA | |
| | | CAAAGCCGCGGGAGGAGCAGTACAACAGCACGTACC | |
| | | GGGTGGTCAGCGTCCTCACCGTCCTGCACCAGGACTG | |
| | | GCTGAATGGCAAGGAGTACAAGTGCAAGGTGTCCAA | |
| | | CAAAGCCCTCCCAGCCCCCGAGGAGAAAACCATCTCC | |
| | | AAAGCCAAAGGGCAGCCCCGAGAACCACAGGTGTAC | |
| | | ACCCTGCCCCCATCCCGGGATGAGCTGACCAAGAACC | |
| | | AGGTCAGCCTGACCTGCCTGGTCAAAGGCTTCTATCC | |
| | | CAGCGACATCGCCGTGGAGTGGGAGAGCAATGGGCA | |
| | | GCCGGAGAACAACTACAAGACCACGCCTCCCGTGCT | |
| | | GGACTCCGACGGCTCCTTCTTCCTCTACAGCAAGCTC | |
| | | ACCGTGGACAAGAGCAGGTGGCAGCAGGGGAACGTG | |
| | | TTCTCATGCTCCGTGATGCATGAGGCTCTGCACAACC | |
| | | ACTACACGCAGAAGAGCCTCTCCCTGTCTCCGGGTTA | |
| | | A | |
| I2676 | Light chain | CAAGCCGTGCTGACACAACCCGCCAGCCTCAGCGCCA | 82 |
| | (LCVR- | GCCCCGGCGCTAGCGCTTCTCTGACATGCACACTGAG | |
| | constant | GTCCGGCATCAACGTGGGCACCTATAGAATCTACTGG | |
| | region) | TACCAGCAGAAACCCGGCTCCCCTCCTCAGTATCTGC | |
| | | TGAGGTACAAGTCCGATAGCGACAAGCAGCAAGGCT | |
| | | CCGGCGTGCCTTCTAGATTTAGCGGCAGCAAGGATGC | |
| | | CAGCGCCAATGCCGGCATTCTGCTGATCAGCGGACTG | |
| | | CAGAGCGAGGATGAGGCCGACTACTACTGCATGATCT | |
| | | GGCACTCCAGCGCCAGAAACTGGGTGTTCGGCGGCG | |
| | | GAACCAAGCTGACCGTGCTA | |
| | | GGCCAGCCCAAGGCTGCCCCCTCGGTCACTCTGTTCC | |
| | | CGCCCTCCTCTGAGGAGCTTCAAGCCAACAAGGCCAC | |
| | | ACTGGTGTGTCTCATAAGTGACTTCTACCCGGGAGCC | |
| | | GTGACAGTGGCCTGGAAGGCAGATAGCAGCCCCGTC | |
| | | AAGGCGGGAGTGGAAACCACCACACCCTCCAAACAA | |
| | | AGCAACAACAAGTACGCGGCCAGCAGCTACCTGAGC | |
| | | CTGACGCCCGAGCAGTGGAAGTCCCACAGAAGCTAC | |
| | | AGCTGCCAGGTCACGCATGAAGGGAGCACCGTGGAA | |
| | | AAGACAGTGGCCCCTACAGAATGTTCATAA | |

### EQUIVALENTS

Those skilled in the art will recognize, or be able to ascertain using no more than routine experimentation, many equivalents to the specific embodiments of the invention described herein. Such equivalents are intended to be encompassed by the following claims.

1-82. (canceled)

83. An antibody, or antigen-binding fragment thereof, that binds to C—C Chemokine Receptor 8 (CCR8), wherein the antibody or antigen-binding fragment thereof comprises:
(i) a heavy chain variable region comprising: a CDR1 domain comprising an amino acid sequence selected from a group consisting of SEQ ID NOs: 9, 15, 21, and 27; a CDR2 domain comprising an amino acid sequence selected from a group consisting of SEQ ID NOs: 10, 16, and 28; and a CDR3 domain comprising an amino acid sequence selected from a group consisting of SEQ ID NOs: 11, 17, 23, and 29; and
(ii) a light chain variable region comprising: a CDR1 domain comprising an amino acid sequence selected from a group consisting of SEQ ID NOs: 12, 30, and 42; a CDR2 domain comprising an amino acid sequence selected from a group consisting of SEQ ID NOs: 13, 31, and 37; and a CDR3 domain comprising an amino acid sequence selected from a group consisting of SEQ ID NOs: 14, 20, and 32.

84. The antibody, or antigen-binding fragment thereof, of claim 83, wherein the antibody, or antigen-binding fragment thereof, comprises a heavy chain variable region comprising an amino acid sequence selected from a group consisting of SEQ ID NOs: 45, 46, and 48, and a light chain variable region comprising an amino acid sequence selected from a group consisting of SEQ ID NOs: 51, 52, and 54-56.

85. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO:10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:14.

86. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 15, a CDR2 domain comprising the amino acid sequence set forth in SEQ ID NO:16; and a CDR3 domain comprising the amino acid sequence set forth in SEQ ID NO:17; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:20.

87. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 21, a CDR2 domain comprising the amino acid sequence set forth in SEQ ID NO:16; and a CDR3 domain comprising the amino acid sequence set forth in SEQ ID NO:23; and a light chain variable region having a CDR1 domain comprising the amino acid sequence set forth in SEQ ID NO:12; a CDR2 domain comprising the amino acid sequence set forth in SEQ ID NO:13; and a CDR3 domain comprising the amino acid sequence set forth in SEQ ID NO:20.

88. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 27, a CDR2 domain comprising the amino acid sequence set forth in SEQ ID NO:28; and a CDR3 domain comprising the amino acid sequence set forth in SEQ ID NO:29; and a light chain variable region having a CDR1 domain comprising the amino acid sequence set forth in SEQ ID NO:30; a CDR2 domain comprising the amino acid sequence set forth in SEQ ID NO:31; and a CDR3 domain comprising the amino acid sequence set forth in SEQ ID NO:32.

89. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:12; a CDR2 domain comprising the amino acid sequence set forth in SEQ ID NO:37; and a CDR3 domain comprising the amino acid sequence set forth in SEQ ID NO:14.

90. The antibody, or antigen-binding fragment thereof, of claim 83, wherein the antibody, or antigen-binding fragment thereof, comprises a heavy chain variable region having a CDR1 domain comprising the amino acid sequence of SEQ ID NO: 9, a CDR2 domain comprising the amino acid sequence set forth in SEQ ID NO. 10; and a CDR3 domain comprising the amino acid sequence set forth in SEQ ID NO:11; and a light chain variable region having a CDR1 domain comprising the amino acid sequence set forth in SEQ ID NO:42; a CDR2 domain comprising the amino acid sequence set forth in SEQ ID NO:13; and a CDR3 domain comprising the amino acid sequence set forth in SEQ ID NO:14.

91. The antibody, or antigen-binding fragment thereof, of claim 83, wherein the antibody, or antigen-binding fragment thereof, comprises a heavy chain variable region comprising the amino acid sequence of SEQ ID NO: 45, and a light chain variable region comprising the amino acid sequence set forth in SEQ ID NO:51.

92. The antibody, or antigen-binding fragment thereof, of claim 83, wherein the antibody, or antigen-binding fragment thereof, comprises a heavy chain variable region comprising the amino acid sequence of SEQ ID NO:46, and a light chain variable region comprising the amino acid sequence set forth in SEQ ID NO:52.

93. The antibody, or antigen-binding fragment thereof, of claim 83, wherein the antibody, or antigen-binding fragment thereof, comprises a heavy chain variable region comprising the amino acid sequence of SEQ ID NO:48, and a light chain variable region comprising the amino acid sequence set forth in SEQ ID NO:54.

94. The antibody, or antigen-binding fragment thereof, of claim 83, wherein the antibody, or antigen-binding fragment thereof, comprises a heavy chain variable region comprising the amino acid sequence of SEQ ID NO:45, and a light chain variable region comprising the amino acid sequence set forth in SEQ ID NO:55.

95. The antibody, or antigen-binding fragment thereof, of claim 83, wherein the antibody, or antigen-binding fragment thereof, comprises a heavy chain variable region comprising the amino acid sequence of SEQ ID NO:45, and a light chain variable region comprising the amino acid sequence set forth in SEQ ID NO:56.

96. The antibody, or antigen-binding fragment thereof, of claim 83, wherein the antibody, or antigen-binding fragment thereof, comprises a heavy chain comprising the amino acid sequence of SEQ ID NO:74, and a light chain comprising the amino acid sequence set forth in SEQ ID NO:76.

97. The antibody, or antigen-binding fragment thereof, of claim 83, wherein the antibody, or antigen-binding fragment thereof, comprises a heavy chain comprising the amino acid sequence of SEQ ID NO:75, and a light chain comprising the amino acid sequence set forth in SEQ ID NO:76.

98. The antibody, or antigen-binding fragment thereof, of claim 83, which is an antibody.

99. A pharmaceutical composition comprising the antibody, or antigen-binding fragment thereof, of claim 1, and a pharmaceutically acceptable carrier.

100. A method of treating cancer in a subject, the method comprising administering to the subject a therapeutically effective amount of the pharmaceutical composition of claim 98, thereby treating cancer in the subject.

101. The method of claim 99, wherein the cancer is selected from a group consisting of colon cancer, breast cancer, lung cancer, liver cancer, pancreatic cancer, ovarian cancer, kidney cancer, bladder cancer, colorectal cancer, endometrial cancer, melanoma, squamous cell carcinoma of the head and neck, renal cell carcinoma, hepatocellular carcinoma, malignant glioma, leukemia, lymphoma, and myeloma.

102. A polynucleotide comprising a polynucleotide encoding a polypeptide comprising:
(a) an immunoglobulin light chain comprising the amino acid sequence set forth in SEQ ID NO: 76, and wherein the light chain when paired with a heavy chain comprising the amino acid sequence set forth in SEQ ID NO: 74 binds to CCR8;
(b) an immunoglobulin light chain comprising the amino acid sequence set forth in SEQ ID NO: 76, and wherein the light chain when paired with a heavy chain comprising the amino acid sequence set forth in SEQ ID NO: 75 binds to CCR8;
(c) an immunoglobulin heavy chain comprising the amino acid sequence set forth in SEQ ID NO: 74, and wherein the heavy chain when paired with a light chain comprising the amino acid sequence set forth in SEQ ID NO: 76 binds to CCR8; or
(d) an immunoglobulin heavy chain comprising the amino acid sequence set forth in SEQ ID NO: 75, and wherein the heavy chain when paired with a light chain comprising the amino acid sequence set forth in SEQ ID NO: 76 binds to CCR8.
